SEQUENCE LISTING (54 sequences)

SEQ ID NO: 1 - DNA, 477 bp, Saccharomyces cerevisiae
  CDS (1)..(477)
atggtgacagggggaatggcaagcaagtgggatcagaagggtatggacattgcctatgaggaggcggccttaggttacaaagagggtggtgttcctattggcggatgtcttatcaataacaaagacggaagtgttctcggtcgtggtcacaacatgagatttcaaaagggatccgccacactacatggtgagatctccactttggaaaactgtgggagattagagggcaaagtgtacaaagataccactttgtatacgacgctgtctccatgcgacatgtgtacaggtgccatcatcatgtatggtattccacgctgtgttgtcggtgagaacgttaatttcaaaagtaagggcgagaaatatttacaaactagaggtcacgaggttgttgttgttgacgatgagaggtgtaaaaagatcatgaaacaatttatcgatgaaagacctcaggattggtttgaagatattggtgagtag

SEQ ID NO: 2 - protein, 158 aa, Saccharomyces cerevisiae
MVTGGMASKWDQKGMDIAYEEAALGYKEGGVPIGGCLINNKDGSVLGRGHNMRFQKGSATLHGEISTLENCGRLEGKVYKDTTLYTTLSPCDMCTGAIIMYGIPRCVVGENVNFKSKGEKYLQTRGHEVVVVDDERCKKIMKQFIDERPQDWFEDIGE

SEQ ID NO: 3 - DNA, 477 bp, Artificial Sequence
  Engineered cytosine deaminase
atggtgacagggggaatggcaagcaagtgggatcagaagggtatggacattgcctatgaggaggcgttattaggttacaaagagggtggtgttcctattggcggatgtcttatcaataacaaagacggaagtgttctcggtcgtggtcacaacatgagatttcaaaagggatccgccacactacatggtgagatctccactttggaaaactgtgggagattagagggcaaagtgtacaaagataccactttgtatacgacgctgtctccatgcgacatgtgtacaggtgccatcatcatgtatggtattccacgctgtgtcatcggtgagaacgttaatttcaaaagtaagggcgagaaatatttacaaactagaggtcacgaggttgttgttgttgacgatgagaggtgtaaaaagttaatgaaacaatttatcgatgaaagacctcaggattggtttgaagatattggtgagtag

SEQ ID NO: 4 - protein, 158 aa, Artificial Sequence
  Synthetic Construct
MVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEVVVVDDERCKKLMKQFIDERPQDWFEDIGE

SEQ ID NO: 5 - DNA, 480 bp, Artificial Sequence
  Human codon optimized cytosine deaminase
atggtgaccggcggcatggcctccaagtgggatcaaaagggcatggatatcgcttacgaggaggccgcactgggctacaaggagggcggcgtgcctatcggcggctgtctgatcaacaacaaggacggcagtgtgctgggcaggggccacaacatgaggttccagaagggctccgccaccctgcacggcgagatctccaccctggagaactgtggcaggctggagggcaaggtgtacaaggacaccaccctgtacaccaccctgtccccttgtgacatgtgtaccggcgctatcatcatgtacggcatccctaggtgtgtggtcggcgagaacgtgaacttcaagtccaagggcgagaagtacctgcaaaccaggggccacgaggtggtggttgttgacgatgagaggtgtaagaagatcatgaagcagttcatcgacgagaggcctcaggactggttcgaggatatcggcgagtgataa

SEQ ID NO: 6 - protein, 158 aa, Artificial Sequence; identical to SEQ ID NO: 2
  Synthetic Construct

SEQ ID NO: 7 - DNA, 756 bp, Saccharomyces cerevisiae
  CDS (1)..(756)
atgaacccgttattctttttggcttctccattcttgtaccttacatatcttatatattatccaaacaaagggtctttcgttagcaaacctagaaatctgcaaaaaatgtcttcggaaccatttaagaacgtctacttgctacctcaaacaaaccaattgctgggtttgtacaccatcatcagaaataagaatacaactagacctgatttcattttctactccgatagaatcatcagattgttggttgaagaaggtttgaaccatctacctgtgcaaaagcaaattgtggaaactgacaccaacgaaaacttcgaaggtgtctcattcatgggtaaaatctgtggtgtttccattgtcagagctggtgaatcgatggagcaaggattaagagactgttgtaggtctgtgcgtatcggtaaaattttaattcaaagggacgaggagactgctttaccaaagttattctacgaaaaattaccagaggatatatctgaaaggtatgtcttcctattagacccaatgctggccaccggtggtagtgctatcatggctacagaagtcttgattaagagaggtgttaagccagagagaatttacttcttaaacctaatctgtagtaaggaagggattgaaaaataccatgccgccttcccagaggtcagaattgttactggtgccctcgacagaggtctagatgaaaacaagtatctagttccagggttgggtgactttggtgacagatactactgtgtttaa

SEQ ID NO: 8 - protein, 251 aa, Saccharomyces cerevisiae
MNPLFFLASPFLYLTYLIYYPNKGSFVSKPRNLQKMSSEPFKNVYLLPQTNQLLGLYTIIRNKNTTRPDFIFYSDRIIRLLVEEGLNHLPVQKQIVETDTNENFEGVSFMGKICGVSIVRAGESMEQGLRDCCRSVRIGKILIQRDEETALPKLFYEKLPEDISERYVFLLDPMLATGGSAIMATEVLIKRGVKPERIYFLNLICSKEGIEKYHAAFPEVRIVTGALDRGLDENKYLVPGLGDFGDRYYCV

SEQ ID NO: 9 - DNA, 1443 bp, homo sapiens
  CDS (1)..(1443)
atggctgttgctcgtgctgctcttggtcctcttgttactggtctttatgatgttcaagcttttaaatttggtgattttgttcttaaatctggtctttcttctcctatttatattgatcttcgtggtattgtttctcgtcctcgtcttctttctcaagttgctgatattctttttcaaactgctcaaaatgctggtatttcttttgatactgtttgtggtgttccttatactgctcttcctcttgctactgttatttgttctactaatcaaattcctatgcttattcgtcgtaaagaaactaaagattatggtactaaacgtcttgttgaaggtactattaatcctggtgaaacttgtcttattattgaagatgttgttacttctggttcttctgttcttgaaactgttgaagttcttcaaaaagaaggtcttaaagttactgatgctattgttcttcttgatcgtgaacaaggtggtaaagataaacttcaagctcatggtattcgtcttcattctgtttgtactctttctaaaatgcttgaaattcttgaacaacaaaaaaaagttgatgctgaaactgttggtcgtgttaaacgttttattcaagaaaatgtttttgttgctgctaatcataatggttctcctctttctattaaagaagctcctaaagaactttcttttggtgctcgtgctgaacttcctcgtattcatcctgttgcttctaaacttcttcgtcttatgcaaaaaaaagaaactaatctttgtctttctgctgatgtttctcttgctcgtgaacttcttcaacttgctgatgctcttggtccttctatttgtatgcttaaaactcatgttgatattcttaatgattttactcttgatgttatgaaagaacttattactcttgctaaatgtcatgaatttcttatttttgaagatcgtaaatttgctgatattggtaatactgttaaaaaacaatatgaaggtggtatttttaaaattgcttcttgggctgatcttgttaatgctcatgttgttcctggttctggtgttgttaaaggtcttcaagaagttggtcttcctcttcatcgtggttgtcttcttattgctgaaatgtcttctactggttctcttgctactggtgattatactcgtgctgctgttcgtatggctgaagaacattctgaatttgttgttggttttatttctggttctcgtgtttctatgaaacctgaatttcttcatcttactcctggtgttcaacttgaagctggtggtgataatcttggtcaacaatataattctcctcaagaagttattggtaaacgtggttctgatattattattgttggtcgtggtattatttctgctgctgatcgtcttgaagctgctgaaatgtatcgtaaagctgcttgggaagcttatctttctcgtcttggtgtttaa

SEQ ID NO: 10 - protein, 480 aa, homo sapiens
MAVARAALGPLVTGLYDVQAFKFGDFVLKSGLSSPIYIDLRGIVSRPRLLSQVADILFQTAQNAGISFDTVCGVPYTALPLATVICSTNQIPMLIRRKETKDYGTKRLVEGTINPGETCLIIEDVVTSGSSVLETVEVLQKEGLKVTDAIVLLDREQGGKDKLQAHGIRLHSVCTLSKMLEILEQQKKVDAETVGRVKRFIQENVFVAANHNGSPLSIKEAPKELSFGARAELPRIHPVASKLLRLMQKKETNLCLSADVSLARELLQLADALGPSICMLKTHVDILNDFTLDVMKELITLAKCHEFLIFEDRKFADIGNTVKKQYEGGIFKIASWADLVNAHVVPGSGVVKGLQEVGLPLHRGCLLIAEMSSTGSLATGDYTRAAVRMAEEHSEFVVGFISGSRVSMKPEFLHLTPGVQLEAGGDNLGQQYNSPQEVIGKRGSDIIIVGRGIISAADRLEAAEMYRKAAWEAYLSRLGV

SEQ ID NO: 11 - DNA, 1227 bp, Artificial Sequence
  Fusion construct CDopt-UPRT
atggtgaccggcggcatggcctccaagtgggatcaaaagggcatggatatcgcttacgaggaggccctgctgggctacaaggagggcggcgtgcctatcggcggctgtctgatcaacaacaaggacggcagtgtgctgggcaggggccacaacatgaggttccagaagggctccgccaccctgcacggcgagatctccaccctggagaactgtggcaggctggagggcaaggtgtacaaggacaccaccctgtacaccaccctgtccccttgtgacatgtgtaccggcgctatcatcatgtacggcatccctaggtgtgtgatcggcgagaacgtgaacttcaagtccaagggcgagaagtacctgcaaaccaggggccacgaggtggtggttgttgacgatgagaggtgtaagaagctgatgaagcagttcatcgacgagaggcctcaggactggttcgaggatatcggcgagaacccgttattctttttggcttctccattcttgtaccttacatatcttatatattatccaaacaaagggtctttcgttagcaaacctagaaatctgcaaaaaatgtcttcggaaccatttaagaacgtctacttgctacctcaaacaaaccaattgctgggtttgtacaccatcatcagaaataagaatacaactagacctgatttcattttctactccgatagaatcatcagattgttggttgaagaaggtttgaaccatctacctgtgcaaaagcaaattgtggaaactgacaccaacgaaaacttcgaaggtgtctcattcatgggtaaaatctgtggtgtttccattgtcagagctggtgaatcgatggagcaaggattaagagactgttgtaggtctgtgcgtatcggtaaaattttaattcaaagggacgaggagactgctttaccaaagttattctacgaaaaattaccagaggatatatctgaaaggtatgtcttcctattagacccaatgctggccaccggtggtagtgctatcatggctacagaagtcttgattaagagaggtgttaagccagagagaatttacttcttaaacctaatctgtagtaaggaagggattgaaaaataccatgccgccttcccagaggtcagaattgttactggtgccctcgacagaggtctagatgaaaacaagtatctagttccagggttgggtgactttggtgacagatactactgtgtttaa

SEQ ID NO: 12 - protein, 408 aa, Artificial Sequence
  Synthetic Construct
MVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEVVVVDDERCKKLMKQFIDERPQDWFEDIGENPLFFLASPFLYLTYLIYYPNKGSFVSKPRNLQKMSSEPFKNVYLLPQTNQLLGLYTIIRNKNTTRPDFIFYSDRIIRLLVEEGLNHLPVQKQIVETDTNENFEGVSFMGKICGVSIVRAGESMEQGLRDCCRSVRIGKILIQRDEETALPKLFYEKLPEDISERYVFLLDPMLATGGSAIMATEVLIKRGVKPERIYFLNLICSKEGIEKYHAAFPEVRIVTGALDRGLDENKYLVPGLGDFGDRYYCV

SEQ ID NO: 13 - DNA, 1287 bp, Artificial Sequence
  Fusion construction - CDopt - linker - UPRT
atggtgaccggcggcatggcctccaagtgggatcaaaagggcatggatatcgcttacgaggaggccctgctgggctacaaggagggcggcgtgcctatcggcggctgtctgatcaacaacaaggacggcagtgtgctgggcaggggccacaacatgaggttccagaagggctccgccaccctgcacggcgagatctccaccctggagaactgtggcaggctggagggcaaggtgtacaaggacaccaccctgtacaccaccctgtccccttgtgacatgtgtaccggcgctatcatcatgtacggcatccctaggtgtgtgatcggcgagaacgtgaacttcaagtccaagggcgagaagtacctgcaaaccaggggccacgaggtggtggttgttgacgatgagaggtgtaagaagctgatgaagcagttcatcgacgagaggcctcaggactggttcgaggatatcggcgagtccggcggcggcgcctccggcggcggcgcctccggcggcggcgcctccggcggcggcgccaacccgttattctttttggcttctccattcttgtaccttacatatcttatatattatccaaacaaagggtctttcgttagcaaacctagaaatctgcaaaaaatgtcttcggaaccatttaagaacgtctacttgctacctcaaacaaaccaattgctgggtttgtacaccatcatcagaaataagaatacaactagacctgatttcattttctactccgatagaatcatcagattgttggttgaagaaggtttgaaccatctacctgtgcaaaagcaaattgtggaaactgacaccaacgaaaacttcgaaggtgtctcattcatgggtaaaatctgtggtgtttccattgtcagagctggtgaatcgatggagcaaggattaagagactgttgtaggtctgtgcgtatcggtaaaattttaattcaaagggacgaggagactgctttaccaaagttattctacgaaaaattaccagaggatatatctgaaaggtatgtcttcctattagacccaatgctggccaccggtggtagtgctatcatggctacagaagtcttgattaagagaggtgttaagccagagagaatttacttcttaaacctaatctgtagtaaggaagggattgaaaaataccatgccgccttcccagaggtcagaattgttactggtgccctcgacagaggtctagatgaaaacaagtatctagttccagggttgggtgactttggtgacagatactactgtgtttaa

SEQ ID NO: 14 - protein, 428 aa, Artificial Sequence
  Synthetic Construct
MVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEVVVVDDERCKKLMKQFIDERPQDWFEDIGESGGGASGGGASGGGASGGGANPLFFLASPFLYLTYLIYYPNKGSFVSKPRNLQKMSSEPFKNVYLLPQTNQLLGLYTIIRNKNTTRPDFIFYSDRIIRLLVEEGLNHLPVQKQIVETDTNENFEGVSFMGKICGVSIVRAGESMEQGLRDCCRSVRIGKILIQRDEETALPKLFYEKLPEDISERYVFLLDPMLATGGSAIMATEVLIKRGVKPERIYFLNLICSKEGIEKYHAAFPEVRIVTGALDRGLDENKYLVPGLGDFGDRYYCV

SEQ ID NO: 15 - DNA, 1200 bp, Artificial Sequence
  Fusion Construct - CDopt3 - OPRT
atggtgaccggcggcatggcctccaagtgggatcaaaagggcatggatatcgcttacgaggaggccctgctgggctacaaggagggcggcgtgcctatcggcggctgtctgatcaacaacaaggacggcagtgtgctgggcaggggccacaacatgaggttccagaagggctccgccaccctgcacggcgagatctccaccctggagaactgtggcaggctggagggcaaggtgtacaaggacaccaccctgtacaccaccctgtccccttgtgacatgtgtaccggcgctatcatcatgtacggcatccctaggtgtgtgatcggcgagaacgtgaacttcaagtccaagggcgagaagtacctgcaaaccaggggccacgaggtggtggttgttgacgatgagaggtgtaagaagctgatgaagcagttcatcgacgagaggcctcaggactggttcgaggatatcggcgaggcggtcgctcgtgcagctttggggccattggtgacgggtctgtacgacgtgcaggctttcaagtttggggacttcgtgctgaagagcgggctttcctcccccatctacatcgatctgcggggcatcgtgtctcgaccgcgtcttctgagtcaggttgcagatattttattccaaactgcccaaaatgcaggcatcagttttgacaccgtgtgtggagtgccttatacagctttgccattggctacagttatctgttcaaccaatcaaattccaatgcttattagaaggaaagaaacaaaggattatggaactaagcgtcttgtagaaggaactattaatccaggagaaacctgtttaatcattgaagatgttgtcaccagtggatctagtgttttggaaactgttgaggttcttcagaaggagggcttgaaggtcactgatgccatagtgctgttggacagagagcagggaggcaaggacaagttgcaggcgcacgggatccgcctccactcagtgtgtacattgtccaaaatgctggagattctcgagcagcagaaaaaagttgatgctgagacagttgggagagtgaagaggtttattcaggagaatgtctttgtggcagcgaatcataatggttctcccctttctataaaggaagcacccaaagaactcagcttcggtgcacgtgcagagctgcccaggatccacccagttgcatcgaagtaa

SEQ ID NO: 16 - protein, 399 aa, Artificial Sequence
  Synthetic Construct
MVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEVVVVDDERCKKLMKQFIDERPQDWFEDIGEAVARAALGPLVTGLYDVQAFKFGDFVLKSGLSSPIYIDLRGIVSRPRLLSQVADILFQTAQNAGISFDTVCGVPYTALPLATVICSTNQIPMLIRRKETKDYGTKRLVEGTINPGETCLIIEDVVTSGSSVLETVEVLQKEGLKVTDAIVLLDREQGGKDKLQAHGIRLHSVCTLSKMLEILEQQKKVDAETVGRVKRFIQENVFVAANHNGSPLSIKEAPKELSFGARAELPRIHPVASK

SEQ ID NO: 17 - DNA, 1260 bp, Artificial Sequence
  Fusion Construct - CDopt3 - linker - OPRT
atggtgaccggcggcatggcctccaagtgggatcaaaagggcatggatatcgcttacgaggaggccctgctgggctacaaggagggcggcgtgcctatcggcggctgtctgatcaacaacaaggacggcagtgtgctgggcaggggccacaacatgaggttccagaagggctccgccaccctgcacggcgagatctccaccctggagaactgtggcaggctggagggcaaggtgtacaaggacaccaccctgtacaccaccctgtccccttgtgacatgtgtaccggcgctatcatcatgtacggcatccctaggtgtgtgatcggcgagaacgtgaacttcaagtccaagggcgagaagtacctgcaaaccaggggccacgaggtggtggttgttgacgatgagaggtgtaagaagctgatgaagcagttcatcgacgagaggcctcaggactggttcgaggatatcggcgagtccggcggcggcgcctccggcggcggcgcctccggcggcggcgcctccggcggcggcgccgcggtcgctcgtgcagctttggggccattggtgacgggtctgtacgacgtgcaggctttcaagtttggggacttcgtgctgaagagcgggctttcctcccccatctacatcgatctgcggggcatcgtgtctcgaccgcgtcttctgagtcaggttgcagatattttattccaaactgcccaaaatgcaggcatcagttttgacaccgtgtgtggagtgccttatacagctttgccattggctacagttatctgttcaaccaatcaaattccaatgcttattagaaggaaagaaacaaaggattatggaactaagcgtcttgtagaaggaactattaatccaggagaaacctgtttaatcattgaagatgttgtcaccagtggatctagtgttttggaaactgttgaggttcttcagaaggagggcttgaaggtcactgatgccatagtgctgttggacagagagcagggaggcaaggacaagttgcaggcgcacgggatccgcctccactcagtgtgtacattgtccaaaatgctggagattctcgagcagcagaaaaaagttgatgctgagacagttgggagagtgaagaggtttattcaggagaatgtctttgtggcagcgaatcataatggttctcccctttctataaaggaagcacccaaagaactcagcttcggtgcacgtgcagagctgcccaggatccacccagttgcatcgaagtaa

SEQ ID NO: 18 - protein, 419 aa, Artificial Sequence
  Synthetic Construct
MVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEVVVVDDERCKKLMKQFIDERPQDWFEDIGESGGGASGGGASGGGASGGGAAVARAALGPLVTGLYDVQAFKFGDFVLKSGLSSPIYIDLRGIVSRPRLLSQVADILFQTAQNAGISFDTVCGVPYTALPLATVICSTNQIPMLIRRKETKDYGTKRLVEGTINPGETCLIIEDVVTSGSSVLETVEVLQKEGLKVTDAIVLLDREQGGKDKLQAHGIRLHSVCTLSKMLEILEQQKKVDAETVGRVKRFIQENVFVAANHNGSPLSIKEAPKELSFGARAELPRIHPVASK

SEQ ID NO: 19 - DNA, 11892 bp, Artificial Sequence
  RCR Vector - pAC3-yCD2
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtccaaaaatcccgatcgttttggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgagaatatgggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccaaacctaaacctcaagttctttctgacagtggggggccgctcatcgacctacttacagaagaccccccgccttatagggacccaagaccacccccttccgacagggacggaaatggtggagaagcgacccctgcgggagaggcaccggacccctccccaatggcatctcgcctacgtgggagacgggagccccctgtggccgactccactacctcgcaggcattccccctccgcgcaggaggaaacggacagcttcaatactggccgttctcctcttctgacctttacaactggaaaaataataacccttctttttctgaagatccaggtaaactgacagctctgatcgagtctgttctcatcacccatcagcccacctgggacgactgtcagcagctgttggggactctgctgaccggagaagaaaaacaacgggtgctcttagaggctagaaaggcggtgcggggcgatgatgggcgccccactcaactgcccaatgaagtcgatgccgcttttcccctcgagcgcccagactgggattacaccacccaggcaggtaggaaccacctagtccactatcgccagttgctcctagcgggtctccaaaacgcgggcagaagccccaccaatttggccaaggtaaaaggaataacacaagggcccaatgagtctccctcggccttcctagagagacttaaggaagcctatcgcaggtacactccttatgaccctgaggacccagggcaagaaactaatgtgtctatgtctttcatttggcagtctgccccagacattgggagaaagttagagaggttagaagatttaaaaaacaagacgcttggagatttggttagagaggcagaaaagatctttaataaacgagaaaccccggaagaaagagaggaacgtatcaggagagaaacagaggaaaaagaagaacgccgtaggacagaggatgagcagaaagagaaagaaagagatcgtaggagacatagagagatgagcaagctattggccactgtcgttagtggacagaaacaggatagacagggaggagaacgaaggaggtcccaactcgatcgcgaccagtgtgcctactgcaaagaaaaggggcactgggctaaagattgtcccaagaaaccacgaggacctcggggaccaagaccccagacctccctcctgaccctagatgactagggaggtcagggtcaggagcccccccctgaacccaggataaccctcaaagtcggggggcaacccgtcaccttcctggtagatactggggcccaacactccgtgctgacccaaaatcctggacccctaagtgataagtctgcctgggtccaaggggctactggaggaaagcggtatcgctggaccacggatcgcaaagtacatctagctaccggtaaggtcacccactctttcctccatgtaccagactgtccctatcctctgttaggaagagatttgctgactaaactaaaagcccaaatccactttgagggatcaggagcccaggttatgggaccaatggggcagcccctgcaagtgttgaccctaaatatagaagatgagcatcggctacatgagacctcaaaagagccagatgtttctctagggtccacatggctgtctgattttcctcaggcctgggcggaaaccgggggcatgggactggcagttcgccaagctcctctgatcatacctctgaaagcaacctctacccccgtgtccataaaacaataccccatgtcacaagaagccagactggggatcaagccccacatacagagactgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggctcccaccgtcccaccagtggtacactgtgcttgatttaaaggatgcctttttctgcctgagactccaccccaccagtcagcctctcttcgcctttgagtggagagatccagagatgggaatctcaggacaattgacctggaccagactcccacagggtttcaaaaacagtcccaccctgtttgatgaggcactgcacagagacctagcagacttccggatccagcacccagacttgatcctgctacagtacgtggatgacttactgctggccgccacttctgagctagactgccaacaaggtactcgggccctgttacaaaccctagggaacctcgggtatcgggcctcggccaagaaagcccaaatttgccagaaacaggtcaagtatctggggtatcttctaaaagagggtcagagatggctgactgaggccagaaaagagactgtgatggggcagcctactccgaagacccctcgacaactaagggagttcctagggacggcaggcttctgtcgcctctggatccctgggtttgcagaaatggcagcccccttgtaccctctcaccaaaacggggactctgtttaattggggcccagaccaacaaaaggcctatcaagaaatcaagcaagctcttctaactgccccagccctggggttgccagatttgactaagccctttgaactctttgtcgacgagaagcagggctacgccaaaggtgtcctaacgcaaaaactgggaccttggcgtcggccggtggcctacctgtccaaaaagctagacccagtagcagctgggtggcccccttgcctacggatggtagcagccattgccgtactgacaaaggatgcaggcaagctaaccatgggacagccactagtcattctggccccccatgcagtagaggcactagtcaaacaaccccccgaccgctggctttccaacgcccggatgactcactatcaggccttgcttttggacacggaccgggtccagttcggaccggtggtagccctgaacccggctacgctgctcccactgcctgaggaagggctgcaacacaactgccttgatatcctggccgaagcccacggaacccgacccgacctaacggaccagccgctcccagacgccgaccacacctggtacacggatggaagcagtctcttacaagagggacagcgtaaggcgggagctgcggtgaccaccgagaccgaggtaatctgggctaaagccctgccagccgggacatccgctcagcgggctgaactgatagcactcacccaggccctaaagatggcagaaggtaagaagctaaatgtttatactgatagccgttatgcttttgctactgcccatatccatggagaaatatacagaaggcgtgggttgctcacatcagaaggcaaagagatcaaaaataaagacgagatcttggccctactaaaagccctctttctgcccaaaagacttagcataatccattgtccaggacatcaaaagggacacagcgccgaggctagaggcaaccggatggctgaccaagcggcccgaaaggcagccatcacagagactccagacacctctaccctcctcatagaaaattcatcaccctacacctcagaacattttcattacacagtgactgatataaaggacctaaccaagttgggggccatttatgataaaacaaagaagtattgggtctaccaaggaaaacctgtgatgcctgaccagtttacttttgaattattagactttcttcatcagctgactcacctcagcttctcaaaaatgaaggctctcctagagagaagccacagtccctactacatgctgaaccgggatcgaacactcaaaaatatcactgagacctgcaaagcttgtgcacaagtcaacgccagcaagtctgccgttaaacagggaactagggtccgcgggcatcggcccggcactcattgggagatcgatttcaccgagataaagcccggattgtatggctataaatatcttctagtttttatagataccttttctggctggatagaagccttcccaaccaagaaagaaaccgccaaggtcgtaaccaagaagctactagaggagatcttccccaggttcggcatgcctcaggtattgggaactgacaatgggcctgccttcgtctccaaggtgagtcagacagtggccgatctgttggggattgattggaaattacattgtgcatacagaccccaaagctcaggccaggtagaaagaatgaatagaaccatcaaggagactttaactaaattaacgcttgcaactggctctagagactgggtgctcctactccccttagccctgtaccgagcccgcaacacgccgggcccccatggcctcaccccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgacatggcgcgttcaacgctctcaaaaccccctcaagataagattaacccgtggaagcccttaatagtcatgggagtcctgttaggagtagggatggcagagagcccccatcaggtctttaatgtaacctggagagtcaccaacctgatgactgggcgtaccgccaatgccacctccctcctgggaactgtacaagatgccttcccaaaattatattttgatctatgtgatctggtcggagaggagtgggacccttcagaccaggaaccgtatgtcgggtatggctgcaagtaccccgcagggagacagcggacccggacttttgacttttacgtgtgccctgggcataccgtaaagtcggggtgtgggggaccaggagagggctactgtggtaaatgggggtgtgaaaccaccggacaggcttactggaagcccacatcatcgtgggacctaatctcccttaagcgcggtaacaccccctgggacacgggatgctctaaagttgcctgtggcccctgctacgacctctccaaagtatccaattccttccaaggggctactcgagggggcagatgcaaccctctagtcctagaattcactgatgcaggaaaaaaggctaactgggacgggcccaaatcgtggggactgagactgtaccggacaggaacagatcctattaccatgttctccctgacccggcaggtccttaatgtgggaccccgagtccccatagggcccaacccagtattacccgaccaaagactcccttcctcaccaatagagattgtaccggctccacagccacctagccccctcaataccagttaccccccttccactaccagtacaccctcaacctcccctacaagtccaagtgtcccacagccacccccaggaactggagatagactactagctctagtcaaaggagcctatcaggcgcttaacctcaccaatcccgacaagacccaagaatgttggctgtgcttagtgtcgggacctccttattacgaaggagtagcggtcgtgggcacttataccaatcattccaccgctccggccaactgtacggccacttcccaacataagcttaccctatctgaagtgacaggacagggcctatgcatgggggcagtacctaaaactcaccaggccttatgtaacaccacccaaagcgccggctcaggatcctactaccttgcagcacccgccggaacaatgtgggcttgcagcactggattgactccctgcttgtccaccacggtgctcaatctaaccacagattattgtgtattagttgaactctggcccagagtaatttaccactcccccgattatatgtatggtcagcttgaacagcgtaccaaatataaaagagagccagtatcattgaccctggcccttctactaggaggattaaccatgggagggattgcagctggaatagggacggggaccactgccttaattaaaacccagcagtttgagcagcttcatgccgctatccagacagacctcaacgaagtcgaaaagtcaattaccaacctagaaaagtcactgacctcgttgtctgaagtagtcctacagaaccgcagaggcctagatttgctattcctaaaggagggaggtctctgcgcagccctaaaagaagaatgttgtttttatgcagaccacacggggctagtgagagacagcatggccaaattaagagaaaggcttaatcagagacaaaaactatttgagacaggccaaggatggttcgaagggctgtttaatagatccccctggtttaccaccttaatctccaccatcatgggacctctaatagtactcttactgatcttactctttggaccttgcattctcaatcgattggtccaatttgttaaagacaggatctcagtggtccaggctctggttttgactcagcaatatcaccagctaaaacccatagagtacgagccatgaacgcgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgattataaatggtgaccggcggcatggcctccaagtgggatcaaaagggcatggatatcgcttacgaggaggccctgctgggctacaaggagggcggcgtgcctatcggcggctgtctgatcaacaacaaggacggcagtgtgctgggcaggggccacaacatgaggttccagaagggctccgccaccctgcacggcgagatctccaccctggagaactgtggcaggctggagggcaaggtgtacaaggacaccaccctgtacaccaccctgtccccttgtgacatgtgtaccggcgctatcatcatgtacggcatccctaggtgtgtgatcggcgagaacgtgaacttcaagtccaagggcgagaagtacctgcaaaccaggggccacgaggtggtggttgttgacgatgagaggtgtaagaagctgatgaagcagttcatcgacgagaggcctcaggactggttcgaggatatcggcgagtaagcggccgcagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcattacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattcat

SEQ ID NO: 20 - DNA, 11892 bp, Artificial Sequence
  RCR Vector - pAC3-yCD
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtccaaaaatcccgatcgttttggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgagaatatgggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccaaacctaaacctcaagttctttctgacagtggggggccgctcatcgacctacttacagaagaccccccgccttatagggacccaagaccacccccttccgacagggacggaaatggtggagaagcgacccctgcgggagaggcaccggacccctccccaatggcatctcgcctacgtgggagacgggagccccctgtggccgactccactacctcgcaggcattccccctccgcgcaggaggaaacggacagcttcaatactggccgttctcctcttctgacctttacaactggaaaaataataacccttctttttctgaagatccaggtaaactgacagctctgatcgagtctgttctcatcacccatcagcccacctgggacgactgtcagcagctgttggggactctgctgaccggagaagaaaaacaacgggtgctcttagaggctagaaaggcggtgcggggcgatgatgggcgccccactcaactgcccaatgaagtcgatgccgcttttcccctcgagcgcccagactgggattacaccacccaggcaggtaggaaccacctagtccactatcgccagttgctcctagcgggtctccaaaacgcgggcagaagccccaccaatttggccaaggtaaaaggaataacacaagggcccaatgagtctccctcggccttcctagagagacttaaggaagcctatcgcaggtacactccttatgaccctgaggacccagggcaagaaactaatgtgtctatgtctttcatttggcagtctgccccagacattgggagaaagttagagaggttagaagatttaaaaaacaagacgcttggagatttggttagagaggcagaaaagatctttaataaacgagaaaccccggaagaaagagaggaacgtatcaggagagaaacagaggaaaaagaagaacgccgtaggacagaggatgagcagaaagagaaagaaagagatcgtaggagacatagagagatgagcaagctattggccactgtcgttagtggacagaaacaggatagacagggaggagaacgaaggaggtcccaactcgatcgcgaccagtgtgcctactgcaaagaaaaggggcactgggctaaagattgtcccaagaaaccacgaggacctcggggaccaagaccccagacctccctcctgaccctagatgactagggaggtcagggtcaggagcccccccctgaacccaggataaccctcaaagtcggggggcaacccgtcaccttcctggtagatactggggcccaacactccgtgctgacccaaaatcctggacccctaagtgataagtctgcctgggtccaaggggctactggaggaaagcggtatcgctggaccacggatcgcaaagtacatctagctaccggtaaggtcacccactctttcctccatgtaccagactgtccctatcctctgttaggaagagatttgctgactaaactaaaagcccaaatccactttgagggatcaggagcccaggttatgggaccaatggggcagcccctgcaagtgttgaccctaaatatagaagatgagcatcggctacatgagacctcaaaagagccagatgtttctctagggtccacatggctgtctgattttcctcaggcctgggcggaaaccgggggcatgggactggcagttcgccaagctcctctgatcatacctctgaaagcaacctctacccccgtgtccataaaacaataccccatgtcacaagaagccagactggggatcaagccccacatacagagactgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggctcccaccgtcccaccagtggtacactgtgcttgatttaaaggatgcctttttctgcctgagactccaccccaccagtcagcctctcttcgcctttgagtggagagatccagagatgggaatctcaggacaattgacctggaccagactcccacagggtttcaaaaacagtcccaccctgtttgatgaggcactgcacagagacctagcagacttccggatccagcacccagacttgatcctgctacagtacgtggatgacttactgctggccgccacttctgagctagactgccaacaaggtactcgggccctgttacaaaccctagggaacctcgggtatcgggcctcggccaagaaagcccaaatttgccagaaacaggtcaagtatctggggtatcttctaaaagagggtcagagatggctgactgaggccagaaaagagactgtgatggggcagcctactccgaagacccctcgacaactaagggagttcctagggacggcaggcttctgtcgcctctggatccctgggtttgcagaaatggcagcccccttgtaccctctcaccaaaacggggactctgtttaattggggcccagaccaacaaaaggcctatcaagaaatcaagcaagctcttctaactgccccagccctggggttgccagatttgactaagccctttgaactctttgtcgacgagaagcagggctacgccaaaggtgtcctaacgcaaaaactgggaccttggcgtcggccggtggcctacctgtccaaaaagctagacccagtagcagctgggtggcccccttgcctacggatggtagcagccattgccgtactgacaaaggatgcaggcaagctaaccatgggacagccactagtcattctggccccccatgcagtagaggcactagtcaaacaaccccccgaccgctggctttccaacgcccggatgactcactatcaggccttgcttttggacacggaccgggtccagttcggaccggtggtagccctgaacccggctacgctgctcccactgcctgaggaagggctgcaacacaactgccttgatatcctggccgaagcccacggaacccgacccgacctaacggaccagccgctcccagacgccgaccacacctggtacacggatggaagcagtctcttacaagagggacagcgtaaggcgggagctgcggtgaccaccgagaccgaggtaatctgggctaaagccctgccagccgggacatccgctcagcgggctgaactgatagcactcacccaggccctaaagatggcagaaggtaagaagctaaatgtttatactgatagccgttatgcttttgctactgcccatatccatggagaaatatacagaaggcgtgggttgctcacatcagaaggcaaagagatcaaaaataaagacgagatcttggccctactaaaagccctctttctgcccaaaagacttagcataatccattgtccaggacatcaaaagggacacagcgccgaggctagaggcaaccggatggctgaccaagcggcccgaaaggcagccatcacagagactccagacacctctaccctcctcatagaaaattcatcaccctacacctcagaacattttcattacacagtgactgatataaaggacctaaccaagttgggggccatttatgataaaacaaagaagtattgggtctaccaaggaaaacctgtgatgcctgaccagtttacttttgaattattagactttcttcatcagctgactcacctcagcttctcaaaaatgaaggctctcctagagagaagccacagtccctactacatgctgaaccgggatcgaacactcaaaaatatcactgagacctgcaaagcttgtgcacaagtcaacgccagcaagtctgccgttaaacagggaactagggtccgcgggcatcggcccggcactcattgggagatcgatttcaccgagataaagcccggattgtatggctataaatatcttctagtttttatagataccttttctggctggatagaagccttcccaaccaagaaagaaaccgccaaggtcgtaaccaagaagctactagaggagatcttccccaggttcggcatgcctcaggtattgggaactgacaatgggcctgccttcgtctccaaggtgagtcagacagtggccgatctgttggggattgattggaaattacattgtgcatacagaccccaaagctcaggccaggtagaaagaatgaatagaaccatcaaggagactttaactaaattaacgcttgcaactggctctagagactgggtgctcctactccccttagccctgtaccgagcccgcaacacgccgggcccccatggcctcaccccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgacatggcgcgttcaacgctctcaaaaccccctcaagataagattaacccgtggaagcccttaatagtcatgggagtcctgttaggagtagggatggcagagagcccccatcaggtctttaatgtaacctggagagtcaccaacctgatgactgggcgtaccgccaatgccacctccctcctgggaactgtacaagatgccttcccaaaattatattttgatctatgtgatctggtcggagaggagtgggacccttcagaccaggaaccgtatgtcgggtatggctgcaagtaccccgcagggagacagcggacccggacttttgacttttacgtgtgccctgggcataccgtaaagtcggggtgtgggggaccaggagagggctactgtggtaaatgggggtgtgaaaccaccggacaggcttactggaagcccacatcatcgtgggacctaatctcccttaagcgcggtaacaccccctgggacacgggatgctctaaagttgcctgtggcccctgctacgacctctccaaagtatccaattccttccaaggggctactcgagggggcagatgcaaccctctagtcctagaattcactgatgcaggaaaaaaggctaactgggacgggcccaaatcgtggggactgagactgtaccggacaggaacagatcctattaccatgttctccctgacccggcaggtccttaatgtgggaccccgagtccccatagggcccaacccagtattacccgaccaaagactcccttcctcaccaatagagattgtaccggctccacagccacctagccccctcaataccagttaccccccttccactaccagtacaccctcaacctcccctacaagtccaagtgtcccacagccacccccaggaactggagatagactactagctctagtcaaaggagcctatcaggcgcttaacctcaccaatcccgacaagacccaagaatgttggctgtgcttagtgtcgggacctccttattacgaaggagtagcggtcgtgggcacttataccaatcattccaccgctccggccaactgtacggccacttcccaacataagcttaccctatctgaagtgacaggacagggcctatgcatgggggcagtacctaaaactcaccaggccttatgtaacaccacccaaagcgccggctcaggatcctactaccttgcagcacccgccggaacaatgtgggcttgcagcactggattgactccctgcttgtccaccacggtgctcaatctaaccacagattattgtgtattagttgaactctggcccagagtaatttaccactcccccgattatatgtatggtcagcttgaacagcgtaccaaatataaaagagagccagtatcattgaccctggcccttctactaggaggattaaccatgggagggattgcagctggaatagggacggggaccactgccttaattaaaacccagcagtttgagcagcttcatgccgctatccagacagacctcaacgaagtcgaaaagtcaattaccaacctagaaaagtcactgacctcgttgtctgaagtagtcctacagaaccgcagaggcctagatttgctattcctaaaggagggaggtctctgcgcagccctaaaagaagaatgttgtttttatgcagaccacacggggctagtgagagacagcatggccaaattaagagaaaggcttaatcagagacaaaaactatttgagacaggccaaggatggttcgaagggctgtttaatagatccccctggtttaccaccttaatctccaccatcatgggacctctaatagtactcttactgatcttactctttggaccttgcattctcaatcgattggtccaatttgttaaagacaggatctcagtggtccaggctctggttttgactcagcaatatcaccagctaaaacccatagagtacgagccatgaacgcgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgattataaatggtgacagggggaatggcaagcaagtgggatcagaagggtatggacattgcctatgaggaggcggccttaggttacaaagagggtggtgttcctattggcggatgtcttatcaataacaaagacggaagtgttctcggtcgtggtcacaacatgagatttcaaaagggatccgccacactacatggtgagatctccactttggaaaactgtgggagattagagggcaaagtgtacaaagataccactttgtatacgacgctgtctccatgcgacatgtgtacaggtgccatcatcatgtatggtattccacgctgtgttgtcggtgagaacgttaatttcaaaagtaagggcgagaaatatttacaaactagaggtcacgaggttgttgttgttgacgatgagaggtgtaaaaagatcatgaaacaatttatcgatgaaagacctcaggattggtttgaagatattggtgagtaggcggccgcagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcattacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattcat

SEQ ID NO: 21 - DNA, 12007 bp, Artificial Sequence
  RCR Vector - pACE-CD
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtccaaaaatcccgatcgttttggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgagaatatgggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccaaacctaaacctcaagttctttctgacagtggggggccgctcatcgacctacttacagaagaccccccgccttatagggacccaagaccacccccttccgacagggacggaaatggtggagaagcgacccctgcgggagaggcaccggacccctccccaatggcatctcgcctacgtgggagacgggagccccctgtggccgactccactacctcgcaggcattccccctccgcgcaggaggaaacggacagcttcaatactggccgttctcctcttctgacctttacaactggaaaaataataacccttctttttctgaagatccaggtaaactgacagctctgatcgagtctgttctcatcacccatcagcccacctgggacgactgtcagcagctgttggggactctgctgaccggagaagaaaaacaacgggtgctcttagaggctagaaaggcggtgcggggcgatgatgggcgccccactcaactgcccaatgaagtcgatgccgcttttcccctcgagcgcccagactgggattacaccacccaggcaggtaggaaccacctagtccactatcgccagttgctcctagcgggtctccaaaacgcgggcagaagccccaccaatttggccaaggtaaaaggaataacacaagggcccaatgagtctccctcggccttcctagagagacttaaggaagcctatcgcaggtacactccttatgaccctgaggacccagggcaagaaactaatgtgtctatgtctttcatttggcagtctgccccagacattgggagaaagttagagaggttagaagatttaaaaaacaagacgcttggagatttggttagagaggcagaaaagatctttaataaacgagaaaccccggaagaaagagaggaacgtatcaggagagaaacagaggaaaaagaagaacgccgtaggacagaggatgagcagaaagagaaagaaagagatcgtaggagacatagagagatgagcaagctattggccactgtcgttagtggacagaaacaggatagacagggaggagaacgaaggaggtcccaactcgatcgcgaccagtgtgcctactgcaaagaaaaggggcactgggctaaagattgtcccaagaaaccacgaggacctcggggaccaagaccccagacctccctcctgaccctagatgactagggaggtcagggtcaggagcccccccctgaacccaggataaccctcaaagtcggggggcaacccgtcaccttcctggtagatactggggcccaacactccgtgctgacccaaaatcctggacccctaagtgataagtctgcctgggtccaaggggctactggaggaaagcggtatcgctggaccacggatcgcaaagtacatctagctaccggtaaggtcacccactctttcctccatgtaccagactgtccctatcctctgttaggaagagatttgctgactaaactaaaagcccaaatccactttgagggatcaggagcccaggttatgggaccaatggggcagcccctgcaagtgttgaccctaaatatagaagatgagcatcggctacatgagacctcaaaagagccagatgtttctctagggtccacatggctgtctgattttcctcaggcctgggcggaaaccgggggcatgggactggcagttcgccaagctcctctgatcatacctctgaaagcaacctctacccccgtgtccataaaacaataccccatgtcacaagaagccagactggggatcaagccccacatacagagactgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggctcccaccgtcccaccagtggtacactgtgcttgatttaaaggatgcctttttctgcctgagactccaccccaccagtcagcctctcttcgcctttgagtggagagatccagagatgggaatctcaggacaattgacctggaccagactcccacagggtttcaaaaacagtcccaccctgtttgatgaggcactgcacagagacctagcagacttccggatccagcacccagacttgatcctgctacagtacgtggatgacttactgctggccgccacttctgagctagactgccaacaaggtactcgggccctgttacaaaccctagggaacctcgggtatcgggcctcggccaagaaagcccaaatttgccagaaacaggtcaagtatctggggtatcttctaaaagagggtcagagatggctgactgaggccagaaaagagactgtgatggggcagcctactccgaagacccctcgacaactaagggagttcctagggacggcaggcttctgtcgcctctggatccctgggtttgcagaaatggcagcccccttgtaccctctcaccaaaacggggactctgtttaattggggcccagaccaacaaaaggcctatcaagaaatcaagcaagctcttctaactgccccagccctggggttgccagatttgactaagccctttgaactctttgtcgacgagaagcagggctacgccaaaggtgtcctaacgcaaaaactgggaccttggcgtcggccggtggcctacctgtccaaaaagctagacccagtagcagctgggtggcccccttgcctacggatggtagcagccattgccgtactgacaaaggatgcaggcaagctaaccatgggacagccactagtcattctggccccccatgcagtagaggcactagtcaaacaaccccccgaccgctggctttccaacgcccggatgactcactatcaggccttgcttttggacacggaccgggtccagttcggaccggtggtagccctgaacccggctacgctgctcccactgcctgaggaagggctgcaacacaactgccttgatatcctggccgaagcccacggaacccgacccgacctaacggaccagccgctcccagacgccgaccacacctggtacacggatggaagcagtctcttacaagagggacagcgtaaggcgggagctgcggtgaccaccgagaccgaggtaatctgggctaaagccctgccagccgggacatccgctcagcgggctgaactgatagcactcacccaggccctaaagatggcagaaggtaagaagctaaatgtttatactgatagccgttatgcttttgctactgcccatatccatggagaaatatacagaaggcgtgggttgctcacatcagaaggcaaagagatcaaaaataaagacgagatcttggccctactaaaagccctctttctgcccaaaagacttagcataatccattgtccaggacatcaaaagggacacagcgccgaggctagaggcaaccggatggctgaccaagcggcccgaaaggcagccatcacagagactccagacacctctaccctcctcatagaaaattcatcaccctacacctcagaacattttcattacacagtgactgatataaaggacctaaccaagttgggggccatttatgataaaacaaagaagtattgggtctaccaaggaaaacctgtgatgcctgaccagtttacttttgaattattagactttcttcatcagctgactcacctcagcttctcaaaaatgaaggctctcctagagagaagccacagtccctactacatgctgaaccgggatcgaacactcaaaaatatcactgagacctgcaaagcttgtgcacaagtcaacgccagcaagtctgccgttaaacagggaactagggtccgcgggcatcggcccggcactcattgggagatcgatttcaccgagataaagcccggattgtatggctataaatatcttctagtttttatagataccttttctggctggatagaagccttcccaaccaagaaagaaaccgccaaggtcgtaaccaagaagctactagaggagatcttccccaggttcggcatgcctcaggtattgggaactgacaatgggcctgccttcgtctccaaggtgagtcagacagtggccgatctgttggggattgattggaaattacattgtgcatacagaccccaaagctcaggccaggtagaaagaatgaatagaaccatcaaggagactttaactaaattaacgcttgcaactggctctagagactgggtgctcctactccccttagccctgtaccgagcccgcaacacgccgggcccccatggcctcaccccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgacatggcgcgttcaacgctctcaaaaccccctcaagataagattaacccgtggaagcccttaatagtcatgggagtcctgttaggagtagggatggcagagagcccccatcaggtctttaatgtaacctggagagtcaccaacctgatgactgggcgtaccgccaatgccacctccctcctgggaactgtacaagatgccttcccaaaattatattttgatctatgtgatctggtcggagaggagtgggacccttcagaccaggaaccgtatgtcgggtatggctgcaagtaccccgcagggagacagcggacccggacttttgacttttacgtgtgccctgggcataccgtaaagtcggggtgtgggggaccaggagagggctactgtggtaaatgggggtgtgaaaccaccggacaggcttactggaagcccacatcatcgtgggacctaatctcccttaagcgcggtaacaccccctgggacacgggatgctctaaagttgcctgtggcccctgctacgacctctccaaagtatccaattccttccaaggggctactcgagggggcagatgcaaccctctagtcctagaattcactgatgcaggaaaaaaggctaactgggacgggcccaaatcgtggggactgagactgtaccggacaggaacagatcctattaccatgttctccctgacccggcaggtccttaatgtgggaccccgagtccccatagggcccaacccagtattacccgaccaaagactcccttcctcaccaatagagattgtaccggctccacagccacctagccccctcaataccagttaccccccttccactaccagtacaccctcaacctcccctacaagtccaagtgtcccacagccacccccaggaactggagatagactactagctctagtcaaaggagcctatcaggcgcttaacctcaccaatcccgacaagacccaagaatgttggctgtgcttagtgtcgggacctccttattacgaaggagtagcggtcgtgggcacttataccaatcattccaccgctccggccaactgtacggccacttcccaacataagcttaccctatctgaagtgacaggacagggcctatgcatgggggcagtacctaaaactcaccaggccttatgtaacaccacccaaagcgccggctcaggatcctactaccttgcagcacccgccggaacaatgtgggcttgcagcactggattgactccctgcttgtccaccacggtgctcaatctaaccacagattattgtgtattagttgaactctggcccagagtaatttaccactcccccgattatatgtatggtcagcttgaacagcgtaccaaatataaaagagagccagtatcattgaccctggcccttctactaggaggattaaccatgggagggattgcagctggaatagggacggggaccactgccttaattaaaacccagcagtttgagcagcttcatgccgctatccagacagacctcaacgaagtcgaaaagtcaattaccaacctagaaaagtcactgacctcgttgtctgaagtagtcctacagaaccgcagaggcctagatttgctattcctaaaggagggaggtctctgcgcagccctaaaagaagaatgttgtttttatgcagaccacacggggctagtgagagacagcatggccaaattaagagaaaggcttaatcagagacaaaaactatttgagacaggccaaggatggttcgaagggctgtttaatagatccccctggtttaccaccttaatctccaccatcatgggacctctaatagtactcttactgatcttactctttggaccttgcattctcaatcgattagtccaatttgttaaagacaggatatcagtggtccaggctctagttttgactcaacaatatcaccagctgaagcctatagagtacgagccatgacgtacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataataccatggtgacagggggaatggcaagcaagtgggatcagaagggtatggacattgcctatgaggaggcggccttaggttacaaagagggtggtgttcctattggcggatgtcttatcaataacaaagacggaagtgttctcggtcgtggtcacaacatgagatttcaaaagggatccgccacactacatggtgagatctccactttggaaaactgtgggagattagagggcaaagtgtacaaagataccactttgtatacgacgctgtctccatgcgacatgtgtacaggtgccatcatcatgtatggtattccacgctgtgttgtcggtgagaacgttaatttcaaaagtaagggcgagaaatatttacaaactagaggtcacgaggttgttgttgttgacgatgagaggtgtaaaaagatcatgaaacaatttatcgatgaaagacctcaggattggtttgaagatattggtgagtaggcggccgcgccatagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttgtttaaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggctgcctcgcgcgtttcggtgatgacggtgaaaacctctgacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattcat

SEQ ID NO: 22 - DNA, 11893 bp, Artificial Sequence
  RCR Vector - pAC3-yCD2
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtccaaaaatcccgatcgttttggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccaaacctaaacctcaagttctttctgacagtggggggccgctcatcgacctacttacagaagaccccccgccttatagggacccaagaccacccccttccgacagggacggaaatggtggagaagcgacccctgcgggagaggcaccggacccctccccaatggcatctcgcctacgtgggagacgggagccccctgtggccgactccactacctcgcaggcattccccctccgcgcaggaggaaacggacagcttcaatactggccgttctcctcttctgacctttacaactggaaaaataataacccttctttttctgaagatccaggtaaactgacagctctgatcgagtctgtcctcatcacccatcagcccacctgggacgactgtcagcagctgttggggactctgctgaccggagaagaaaaacaacgggtgctcttagaggctagaaaggcggtgcggggcgatgatgggcgccccactcaactgcccaatgaagtcgatgccgcttttcccctcgagcgcccagactgggattacaccacccaggcaggtaggaaccacctagtccactatcgccagttgctcctagcgggtctccaaaacgcgggcagaagccccaccaatttggccaaggtaaaaggaataacacaagggcccaatgagtctccctcggccttcctagagagacttaaggaagcctatcgcaggtacactccttatgaccctgaggacccagggcaagaaactaatgtgtctatgtctttcatttggcagtctgccccagacattgggagaaagttagagaggttagaagatttaaaaaacaagacgcttggagatttggttagagaggcagaaaagatctttaataaacgagaaaccccggaagaaagagaggaacgtatcaggagagaaacagaggaaaaagaagaacgccgtaggacagaggatgagcagaaagagaaagaaagagatcgtaggagacatagagagatgagcaagctattggccactgtcgttagtggacagaaacaggatagacagggaggagaacgaaggaggtcccaactcgatcgcgaccagtgtgcctactgcaaagaaaaggggcactgggctaaagattgtcccaagaaaccacgaggacctcggggaccaagaccccagacctccctcctgaccctagatgactagggaggtcagggtcaggagcccccccctgaacccaggataaccctcaaagtcggggggcaacccgtcaccttcctggtagatactggggcccaacactccgtgctgacccaaaatcctggacccctaagtgataagtctgcctgggtccaaggggctactggaggaaagcggtatcgctggaccacggatcgcaaagtacatctagctaccggtaaggtcacccactctttcctccatgtaccagactgtccctatcctctgttaggaagagatttgctgactaaactaaaagcccaaatccactttgagggatcaggagcccaggttatgggaccaatggggcagcccctgcaagtgttgaccctaaatatagaagatgagtatcggctacatgagacctcaaaagagccagatgtttctctagggtccacatggctgtctgattttcctcaggcctgggcggaaaccgggggcatgggactggcagttcgccaagctcctctgatcatacctctgaaagcaacctctacccccgtgtccataaaacaataccccatgtcacaagaagccagactggggatcaagccccacatacagagactgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggctcccaccgtcccaccagtggtacactgtgcttgatttaaaggatgcctttttctgcctgagactccaccccaccagtcagcctctcttcgcctttgagtggagagatccagagatgggaatctcaggacaattgacctggaccagactcccacagggtttcaaaaacagtcccaccctgtttgatgaggcactgcacagagacctagcagacttccggatccagcacccagacttgatcctgctacagtacgtggatgacttactgctggccgccacttctgagctagactgccaacaaggtactcgggccctgttacaaaccctagggaacctcgggtatcgggcctcggccaagaaagcccaaatttgccagaaacaggtcaagtatctggggtatcttctaaaagagggtcagagatggctgactgaggccagaaaagagactgtgatggggcagcctactccgaagacccctcgacaactaagggagttcctagggacggcaggcttctgtcgcctctggatccctgggtttgcagaaatggcagcccccttgtaccctctcaccaaaacggggactctgtttaattggggcccagaccaacaaaaggcctatcaagaaatcaagcaagctcttctaactgccccagccctggggttgccagatttgactaagccctttgaactctttgtcgacgagaagcagggctacgccaaaggtgtcctaacgcaaaaactgggaccttggcgtcggccggtggcctacctgtccaaaaagctagacccagtagcagctgggtggcccccttgcctacggatggtagcagccattgccgtactgacaaaggatgcaggcaagctaaccatgggacagccactagtcattctggccccccatgcagtagaggcactagtcaaacaaccccccgaccgctggctttccaacgcccggatgactcactatcaggccttgcttttggacacggaccgggtccagttcggaccggtggtagccctgaacccggctacgctgctcccactgcctgaggaagggctgcaacacaactgccttgatatcctggccgaagcccacggaacccgacccgacctaacggaccagccgctcccagacgccgaccacacctggtacacggatggaagcagtctcttacaagagggacagcgtaaggcgggagctgcggtgaccaccgagaccgaggtaatctgggctaaagccctgccagccgggacatccgctcagcgggctgaactgatagcactcacccaggccctaaagatggcagaaggtaagaagctaaatgtttatactgatagccgttatgcttttgctactgcccatatccatggagaaatatacagaaggcgtgggttgctcacatcagaaggcaaagagatcaaaaataaagacgagatcttggccctactaaaagccctctttctgcccaaaagacttagcataatccattgtccaggacatcaaaagggacacagcgccgaggctagaggcaaccggatggctgaccaagcggcccgaaaggcagccatcacagagactccagacacctctaccctcctcatagaaaattcatcaccctacacctcagaacattttcattacacagtgactgatataaaggacctaaccaagttgggggccatttatgataaaacaaagaagtattgggtctaccaaggaaaacctgtgatgcctgaccagtttacttttgaattattagactttcttcatcagctgactcacctcagcttctcaaaaatgaaggctctcctagagagaagccacagtccctactacatgctgaaccgggatcgaacactcaaaaatatcactgagacctgcaaagcttgtgcacaagtcaacgccagcaagtctgccgttaaacagggaactagggtccgcgggcatcggcccggcactcattgggagatcgatttcaccgagataaagcccggattgtatggctataaatatcttctagtttttatagataccttttctggctggatagaagccttcccaaccaagaaagaaaccgccaaggtcgtaaccaagaagctactagaggagatcttccccaggttcggcatgcctcaggtattgggaactgacaatgggcctgccttcgtctccaaggtgagtcagacagtggccgatctgttggggattgattggaaattacattgtgcatacagaccccaaagctcaggccaggtagaaagaatgaatagaaccatcaaggagactttaactaaattaacgcttgcaactggctctagagactgggtgctcctactccccttagccctgtaccgagcccgcaacacgccgggcccccatggcctcaccccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgacatggcgcgttcaacgctctcaaaaccccctcaagataagattaacccgtggaagcccttaatagtcatgggagtcctgttaggagtagggatggcagagagcccccatcaggtctttaatgtaacctggagagtcaccaacctgatgactgggcgtaccgccaatgccacctccctcctgggaactgtacaagatgccttcccaaaattatattttgatctatgtgatctggtcggagaggagtgggacccttcagaccaggaaccgtatgtcgggtatggctgcaagtaccccgcagggagacagcggacccggacttttgacttttacgtgtgccctgggcataccgtaaagtcggggtgtgggggaccaggagagggctactgtggtaaatgggggtgtgaaaccaccggacaggcttactggaagcccacatcatcgtgggacctaatctcccttaagcgcggtaacaccccctgggacacgggatgctctaaagttgcctgtggcccctgctacgacctctccaaagtatccaattccttccaaggggctactcgagggggcagatgcaaccctctagtcctagaattcactgatgcaggaaaaaaggctaactgggacgggcccaaatcgtggggactgagactgtaccggacaggaacagatcctattaccatgttctccctgacccggcaggtccttaatgtgggaccccgagtccccatagggcccaacccagtattacccgaccaaagactcccttcctcaccaatagagattgtaccggctccacagccacctagccccctcaataccagttaccccccttccactaccagtacaccctcaacctcccctacaagtccaagtgtcccacagccacccccaggaactggagatagactactagctctagtcaaaggagcctatcaggcgcttaacctcaccaatcccgacaagacccaagaatgttggctgtgcttagtgtcgggacctccttattacgaaggagtagcggtcgtgggcacttataccaatcattccaccgctccggccaactgtacggccacttcccaacataagcttaccctatctgaagtgacaggacagggcctatgcatgggggcagtacctaaaactcaccaggccttatgtaacaccacccaaagcgccggctcaggatcctactaccttgcagcacccgccggaacaatgtgggcttgcagcactggattgactccctgcttgtccaccacggtgctcaatctaaccacagattattgtgtattagttgaactctggcccagagtaatttaccactcccccgattatatgtatggtcagcttgaacagcgtaccaaatataaaagagagccagtatcattgaccctggcccttctactaggaggattaaccatgggagggattgcagctggaatagggacggggaccactgccttaattaaaacccagcagtttgagcagcttcatgccgctatccagacagacctcaacgaagtcgaaaagtcaattaccaacctagaaaagtcactgacctcgttgtctgaagtagtcctacagaaccgcagaggcctagatttgctattcctaaaggagggaggtctctgcgcagccctaaaagaagaatgttgtttttatgcagaccacacggggctagtgagagacagcatggccaaattaagagaaaggcttaatcagagacaaaaactatttgagacaggccaaggatggttcgaagggctgtttaatagatccccctggtttaccaccttaatctccaccatcatgggacctctaatagtactcttactgatcttactctttggaccttgcattctcaatcgattggtccaatttgttaaagacaggatctcagtggtccaggctctggttttgactcagcaatatcaccagctaaaacccatagagtacgagccatgaacgcgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaanncgtctaggccccccgaaccacggggacgtggttttcctttgaaaaacacgattataaatggtgaccggcggcatggcctccaagtgggatcaaaagggcatggatatcgcttacgaggaggccctgctgggctacaaggagggcggcgtgcctatcggcggctgtctgatcaacaacaaggacggcagtgtgctgggcaggggccacaacatgaggttccagaagggctccgccaccctgcacggcgagatctccaccctggagaactgtggcaggctggagggcaaggtgtacaaggacaccaccctgtacaccaccctgtccccttgtgacatgtgtaccggcgctatcatcatgtacggcatccctaggtgtgtgatcggcgagaacgtgaacttcaagtccaagggcgagaagtacctgcaaaccaggggccacgaggtggtggttgttgacgatgagaggtgtaagaagctgatgaagcagttcatcgacgagaggcctcaggactggttcgaggatatcggcgagtaagcggccgcagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcattacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattccat

SEQ ID NO: 23 - DNA, 4473 bp, Homo sapiens
  CDS (175)..(3942)
aaggggaggtaaccctggcccctttggtcggggccccgggcagccgcgcgccccttcccacggggccctttactgcgccgcgcgcccggcccccacccctcgcagcaccccgcgccccgcgccctcccagccgggtccagccggagccatggggccggagccgcagtgagcaccatggagctggcggccttgtgccgctgggggctcctcctcgccctcttgccccccggagccgcgagcacccaagtgtgcaccggcacagacatgaagctgcggctccctgccagtcccgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaaggacatcttccacaagaacaaccagctggctctcacactgatagacaccaaccgctctcgggcctgccacccctgttctccgatgtgtaagggctcccgctgctggggagagagttctgaggattgtcagagcctgacgcgcactgtctgtgccggtggctgtgcccgctgcaaggggccactgcccactgactgctgccatgagcagtgtgctgccggctgcacgggccccaagcactctgactgcctggcctgcctccacttcaaccacagtggcatctgtgagctgcactgcccagccctggtcacctacaacacagacacgtttgagtccatgcccaatcccgagggccggtatacattcggcgccagctgtgtgactgcctgtccctacaactacctttctacggacgtgggatcctgcaccctcgtctgccccctgcacaaccaagaggtgacagcagaggatggaacacagcggtgtgagaagtgcagcaagccctgtgcccgagtgtgctatggtctgggcatggagcacttgcgagaggtgagggcagttaccagtgccaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcactgctggggtccagggcccacccagtgtgtcaactgcagccagttccttcggggccaggagtgcgtggaggaatgccgagtactgcaggggctccccagggagtatgtgaatgccaggcactgtttgccgtgccaccctgagtgtcagccccagaatggctcagtgacctgttttggaccggaggctgaccagtgtgtggcctgtgcccactataaggaccctcccttctgcgtggcccgctgccccagcggtgtgaaacctgacctctcctacatgcccatctggaagtttccagatgaggagggcgcatgccagccttgccccatcaactgcacccactcctgtgtggacctggatgacaagggctgccccgccgagcagagagccagccctctgacgtccatcatctctgcggtggttggcattctgctggtcgtggtcttgggggtggtctttgggatcctcatcaagcgacggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagaccatgtccgggaaaaccgcggacgcctgggctcccaggacctgctgaactggtgtatgcagattgccaaggggatgagctacctggaggatgtgcggctcgtacacagggacttggccgctcggaacgtgctggtcaagagtcccaaccatgtcaaaattacagacttcgggctggctcggctgctggacattgacgagacagagtaccatgcagatgggggcaaggtgcccatcaagtggatggcgctggagtccattctccgccggcggttcacccaccagagtgatgtgtggagttatggtgtgactgtgtgggagctgatgacttttggggccaaaccttacgatgggatcccagcccgggagatccctgacctgctggaaaagggggagcggctgccccagccccccatctgcaccattgatgtctacatgatcatggtcaaatgttggatgattgactctgaatgtcggccaagattccgggagttggtgtctgaattctcccgcatggccagggacccccagcgctttgtggtcatccagaatgaggacttgggcccagccagtcccttggacagcaccttctaccgctcactgctggaggacgatgacatgggggacctggtggatgctgaggagtatctggtaccccagcagggcttcttctgtccagaccctgccccgggcgctgggggcatggtccaccacaggcaccgcagctcatctaccaggagtggcggtggggacctgacactagggctggagccctctgaagaggaggcccccaggtctccactggcaccctccgaaggggctggctccgatgtatttgatggtgacctgggaatgggggcagccaaggggctgcaaagcctccccacacatgaccccagccctctacagcggtacagtgaggaccccacagtacccctgccctctgagactgatggctacgttgcccccctgacctgcagcccccagcctgaatatgtgaaccagccagatgttcggccccagcccccttcgccccgagagggccctctgcctgctgcccgacctgctggtgccactctggaaaggcccaagactctctccccagggaagaatggggtcgtcaaagacgtttttgcctttgggggtgccgtggagaaccccgagtacttgacaccccagggaggagctgcccctcagccccaccctcctcctgccttcagcccagccttcgacaacctctattactgggaccaggacccaccagagcggggggctccacccagcaccttcaaagggacacctacggcagagaacccagagtacctgggtctggacgtgccagtgtgaaccagaaggccaagtccgcagaagccctgatgtgtcctcagggagcagggaaggcctgacttctgctggcatcaagaggtgggagggccctccgaccacttccaggggaacctgccatgccaggaacctgtcctaaggaaccttccttcctgcttgagttcccagatggctggaaggggtccagcctcgttggaagaggaacagcactggggagtctttgtggattctgaggccctgcccaatgagactctagggtccagtggatgccacagcccagcttggccctttccttccagatcctgggtactgaaagccttagggaagctggcctgagaggggaagcggccctaagggagtgtctaagaacaaaagcgacccattcagagactgtccctgaaacctagtactgccccccatgaggaaggaacagcaatggtgtcagtatccaggctttgtacagagtgcttttctgtttagtttttactttttttgttttgtttttttaaagatgaaataaagacccagggggag

SEQ ID NO: 24 - protein, 1255 aa, Homo sapiens [sequence omitted]

SEQ ID NO: 25 - DNA, 1212 bp, Homo sapiens
  CDS (1)..(1212)
atgacagccatcatcaaagagatcgttagcagaaacaaaaggagatatcaagaggatggattcgacttagacttgacctatatttatccaaacattattgctatgggatttcctgcagaaagacttgaaggcgtatacaggaacaatattgatgatgtagtaaggtttttggattcaaagcataaaaaccattacaagatatacaatctttgtgctgaaagacattatgacaccgccaaatttaattgcagagttgcacaatatccttttgaagaccataacccaccacagctagaacttatcaaacccttttgtgaagatcttgaccaatggctaagtgaagatgacaatcatgttgcagcaattcactgtaaagctggaaagggacgaactggtgtaatgatatgtgcatatttattacatcggggcaaatttttaaaggcacaagaggccctagatttctatggggaagtaaggaccagagacaaaaagggagtaactattcccagtcagaggcgctatgtgtattattatagctacctgttaaagaatcatctggattatagaccagtggcactgttgtttcacaagatgatgtttgaaactattccaatgttcagtggcggaacttgcaatcctcagtttgtggtctgccagctaaaggtgaagatatattcctccaattcaggacccacacgacgggaagacaagttcatgtactttgagttccctcagccgttacctgtgtgtggtgatatcaaagtagagttcttccacaaacagaacaagatgctaaaaaaggacaaaatgtttcacttttgggtaaatacattcttcataccaggaccagaggaaacctcagaaaaagtagaaaatggaagtctatgtgatcaagaaatcgatagcatttgcagtatagagcgtgcagataatgacaaggaatatctagtacttactttaacaaaaaatgatcttgacaaagcaaataaagacaaagccaaccgatacttttctccaaattttaaggtgaagctgtacttcacaaaaacagtagaggagccgtcaaatccagaggctagcagttcaacttctgtaacaccagatgttagtgacaatgaacctgatcattatagatattctgacaccactgactctgatccagagaatgaaccttttgatgaagatcagcatacacaaattacaaaagtctga

SEQ ID NO: 26 - protein, 403 aa, Homo sapiens [sequence omitted]

SEQ ID NO: 27 - DNA, 597 bp, Homo sapiens
  CDS (1)..(597)
atgtcaaacgtgcgagtgtctaacgggagccctagcctggagcggatggacgccaggcaggcggagcaccccaagccctcggcctgcaggaacctcttcggcccggtggaccacgaagagttaacccgggacttggagaagcactgcagagacatggaagaggcgagccagcgcaagtggaatttcgattttcagaatcacaaacccctagagggcaagtacgagtggcaagaggtggagaagggcagcttgcccgagttctactacagacccccgcggccccccaaaggtgcctgcaaggtgccggcgcaggagagccaggatgtcagcgggagccgcccggcggcgcctttaattggggctccggctaactctgaggacacgcatttggtggacccaaagactgatccgtcggacagccagacggggttagcggagcaatgcgcaggaataaggaagcgacctgcaaccgacgattcttctactcaaaacaaaagagccaacagaacagaagaaaatgtttcagacggttccccaaatgccggttctgtggagcagacgcccaagaagcctggcctcagaagacgtcaaacgtaa

SEQ ID NO: 28 - protein, 198 aa, Homo sapiens
MSNVRVSNGSPSLERMDARQAEHPKPSACRNLFGPVDHEELTRDLEKHCRDMEEASQRKWNFDFQNHKPLEGKYEWQEVEKGSLPEFYYRPPRPPKGACKVPAQESQDVSGSRPAAPLIGAPANSEDTHLVDPKTDPSDSQTGLAEQCAGIRKRPATDDSSTQNKRANRTEENVSDGSPNAGSVEQTPKKPGLRRRQT

SEQ ID NO: 29 - DNA, 894 bp, Homo sapiens
  CDS (1)..(894)
atgacaacacccagaaattcagtaaatgggactttcccggcagagccaatgaaaggccctattgctatgcaatctggtccaaaaccactcttcaggaggatgtcttcactggtgggccccacgcaaagcttcttcatgagggaatctaagactttgggggctgtccagattatgaatgggctcttccacattgccctggggggtcttctgatgatcccagcagggatctatgcacccatctgtgtgactgtgtggtaccctctctggggaggcattatgtatattatttccggatcactcttggcagcaacggagaaaaactctaggaagtgtttggtcaaaggaaaaatgataatgaattcattgagcctctttgctgccatttctggaatgattctttcaatcatggacatacttaatattaaaatttcccattttttaaaaatggagagtctgaattttattagagctcacacaccatatattaacatatacaactgtgaaccagctaatccctctgagaaaaactccccatctacccaatactgttacagcatacaatctctgttcttgggcattttgtcagtgatgctgatctttgccttcttccaggaacttgtaatagctggcatcgttgagaatgaatggaaaagaacgtgctccagacccaaatctaacatagttctcctgtcagcagaagaaaaaaaagaacagactattgaaataaaagaagaagtggttgggctaactgaaacatcttcccaaccaaagaatgaagaagacattgaaattattccaatccaagaagaggaagaagaagaaacagagacgaactttccagaacctccccaagatcaggaatcctcaccaatagaaaatgacagctctccttaa

SEQ ID NO: 30 - protein, 297 aa, Homo sapiens
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSP

SEQ ID NO: 31 - DNA, 596 bp, homo sapiens
acgcgtactggagtcaatgaaagcaactatttcaaaagatcagattacttaccagtttcactaataaagatttattactttaaacctttatcataaaatgtatgctttgaatactgtgaagtacactgcatataaggagtgtggtatagtataaagaaactttctgcaggtagtaattatagtgaagattttaggtttacaaagccctagctgttttctgtgtagcttttattattcttatgactcttgacaagtttgtagcttcaccatatacatttaatattttgcaataattggccttgttcctgagctgttggattcggggccgtagcactgtctgagaggtttacatttctcacagtgaaccggtctctttttcagctgcttcctggcttctttttactcaggtttccactgcttttttgctttttttaatgctgtatgaaggtgttaacatttgtttatatttttcattaattgtaatacctttaaatcatgcatcatactcagaaatagggattagaatttaagtgacatctttggcctaatataatttacctgttaaaaatttgtgaaagctattgcttagcggccgc

SEQ ID NO: 32 - DNA, 511 bp, Homo sapiens
acgcgtccatgtccgtacctttctagttcataccttcttttaattttttttttcttttcaatttgaagagagtgcttcctctgttcttaaggctagggaaccaaattaggttgtttcaatatcgtgctaaaagatactgcctttagaagaaggctattgacaatccagcgtgtctcggtggaactctgactccatggttcactttcatgatggccacatgcctcctgcccagagcccggcagccactgtgcagtgggaaggggggccgatacactgtacgagagtgagtagcaggtctcacagtgaaccggtctctttccctactgtgtcacactcctaatggaatgccgttatccaaagagcagcacgaacccgacagggctgagtggcttgtgctagggagaggtttgtgtcattcctgctgaccaaactgcaggaaaaactgctaattgtcatgctgaagactgcctgacggggagactctgccttctgtaagtaggtcagcggccgc

SEQ ID NO: 33 - DNA, 203 bp, Homo sapiens
acgcgtaattcatatttgcatgtcgctatgtgttctgggaaatcaccataaacgtgaaatgtctttggatttgggaatcttataagttctgtatgagaccactcggatgagctgttggattcggggccgtagcactgtctgagaggtttacatttctcacagtgaaccggtctctttttcagctgcttcttttttgcggccgc

SEQ ID NO: 34 - DNA, 205 bp, Homo sapiens
gcggccgcaattcatatttgcatgtcgctatgtgttctgggaaatcaccataaacgtgaaatgtctttggatttgggaatcttataagttctgtatgagaccactcggatgagctgttggattcggggccgtagcactgtctgagaggtttacatttctcacagtgaaccggtctctttttcagctgcttcttttttgcggccgc

SEQ ID NO: 35 - DNA, 45 bp, Artificial Sequence
  DNA target sequence of the miR-142-3p
gcggccgcgtcgactccataaagtaggaaacactacagcggccgc

SEQ ID NO: 36 - DNA, 128 bp, Artificial Sequence
  DNA target sequence four time repeat miR-142-3pT4X
gcggccgcgtcgactccataaagtaggaaacactacacgattccataaagtaggaaacactacaaccggttccataaagtaggaaacactacatcactccataaagtaggaaacactacagcggccgc

SEQ ID NO: 37 - DNA, 1131 bp, herpes simplex virus 1
atggcttcgtaccccggccatcagcacgcgtctgcgttcgaccaggctgcgcgttctcgcggccatagcaaccgacgtacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcaggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgttacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgagctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctaccgggacgccctgctgcaacttacctccgggatggtccagacccacgtcaccacccccggctccataccgacgatctgcgacctggcgcgcacgtttgcccgggagatgggggaggctaactaa

SEQ ID NO: 38 - DNA, 499 bp, Homo sapiens
atgaaatatacaagttatatcttggcttttcagctctgcatcgttttgggttctcttggctgttactgccaggaccatatgtaaaagaagcagaaaaccttaagaaatattttaatgcaggtcattcagatgtagcggataatggaactcttttcttaggcattttgaagaattggaaagaggagagtgacagaaaaataatgcagagccaaattgtctccttttacttcaaactttttaaaaactttaaagatgaccagagcatccaaaagagtgtggagaccatcaaggaagacatgaatgtaagtttttcaatagcaacaaaaagaaacgagatgacttcgaaaagctgactaattattcggtaactgacttgaatgtccaacgcaaagcaatacatgaactcatccaagtgatggctgaactgtcgccagcagctaaaacagggaagcgaaaaaggagtcagatgctgtttcgaggtcgaagagcatcccagtaa

SEQ ID NO: 39 - DNA, 468 bp, Mus musculus
atgaacgctacacactgcatcttggctttgcagctcttcctcatggctgtttctggctgttactgccacggcacagtcattgaaagcctagaaagtctgaataactattttaactcaagtggcatagatgtggaagaaaagagtctcttcttggatatctggaggaactggcaaaaggatggtgacatgaaaatcctgcagagccagattatctctttctacctcagactctttgaagtcttgaaagacaatcaggccatcagcaacaacataagcgtcattgaatcacacctgattactaccttcttcagcaacagcaaggcgaaaaaggatgcattcatgagtattgccaagtttgaggtcaacaacccacaggtccagcgccaagcattcaatgagctcatccgagtggtccaccagctgttgccggaatccagcctcaggaagcggaaaaggagtcgctgctga

SEQ ID NO: 40 - DNA, 462 bp, Homo sapiens
atgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttga

SEQ ID NO: 41 - DNA, 544 bp, Encephalomyocarditis virus
  misc_feature (492)..(492): “n” can be present or absent, if present n is “A”
cgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaancgtctaggccccccgaaccacggggacgtggttttcctttgaaaaacacgat

SEQ ID NO: 42 - DNA, 11892 bp, Artificial Sequence
  pAC3-yCD2-6A
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtccaaaaatcccgatcgttttggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccaaacctaaacctcaagttctttctgacagtggggggccgctcatcgacctacttacagaagaccccccgccttatagggacccaagaccacccccttccgacagggacggaaatggtggagaagcgacccctgcgggagaggcaccggacccctccccaatggcatctcgcctacgtgggagacgggagccccctgtggccgactccactacctcgcaggcattccccctccgcgcaggaggaaacggacagcttcaatactggccgttctcctcttctgacctttacaactggaaaaataataacccttctttttctgaagatccaggtaaactgacagctctgatcgagtctgtcctcatcacccatcagcccacctgggacgactgtcagcagctgttggggactctgctgaccggagaagaaaaacaacgggtgctcttagaggctagaaaggcggtgcggggcgatgatgggcgccccactcaactgcccaatgaagtcgatgccgcttttcccctcgagcgcccagactgggattacaccacccaggcaggtaggaaccacctagtccactatcgccagttgctcctagcgggtctccaaaacgcgggcagaagccccaccaatttggccaaggtaaaaggaataacacaagggcccaatgagtctccctcggccttcctagagagacttaaggaagcctatcgcaggtacactccttatgaccctgaggacccagggcaagaaactaatgtgtctatgtctttcatttggcagtctgccccagacattgggagaaagttagagaggttagaagatttaaaaaacaagacgcttggagatttggttagagaggcagaaaagatctttaataaacgagaaaccccggaagaaagagaggaacgtatcaggagagaaacagaggaaaaagaagaacgccgtaggacagaggatgagcagaaagagaaagaaagagatcgtaggagacatagagagatgagcaagctattggccactgtcgttagtggacagaaacaggatagacagggaggagaacgaaggaggtcccaactcgatcgcgaccagtgtgcctactgcaaagaaaaggggcactgggctaaagattgtcccaagaaaccacgaggacctcggggaccaagaccccagacctccctcctgaccctagatgactagggaggtcagggtcaggagcccccccctgaacccaggataaccctcaaagtcggggggcaacccgtcaccttcctggtagatactggggcccaacactccgtgctgacccaaaatcctggacccctaagtgataagtctgcctgggtccaaggggctactggaggaaagcggtatcgctggaccacggatcgcaaagtacatctagctaccggtaaggtcacccactctttcctccatgtaccagactgtccctatcctctgttaggaagagatttgctgactaaactaaaagcccaaatccactttgagggatcaggagcccaggttatgggaccaatggggcagcccctgcaagtgttgaccctaaatatagaagatgagtatcggctacatgagacctcaaaagagccagatgtttctctagggtccacatggctgtctgattttcctcaggcctgggcggaaaccgggggcatgggactggcagttcgccaagctcctctgatcatacctctgaaagcaacctctacccccgtgtccataaaacaataccccatgtcacaagaagccagactggggatcaagccccacatacagagactgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggctcccaccgtcccaccagtggtacactgtgcttgatttaaaggatgcctttttctgcctgagactccaccccaccagtcagcctctcttcgcctttgagtggagagatccagagatgggaatctcaggacaattgacctggaccagactcccacagggtttcaaaaacagtcccaccctgtttgatgaggcactgcacagagacctagcagacttccggatccagcacccagacttgatcctgctacagtacgtggatgacttactgctggccgccacttctgagctagactgccaacaaggtactcgggccctgttacaaaccctagggaacctcgggtatcgggcctcggccaagaaagcccaaatttgccagaaacaggtcaagtatctggggtatcttctaaaagagggtcagagatggctgactgaggccagaaaagagactgtgatggggcagcctactccgaagacccctcgacaactaagggagttcctagggacggcaggcttctgtcgcctctggatccctgggtttgcagaaatggcagcccccttgtaccctctcaccaaaacggggactctgtttaattggggcccagaccaacaaaaggcctatcaagaaatcaagcaagctcttctaactgccccagccctggggttgccagatttgactaagccctttgaactctttgtcgacgagaagcagggctacgccaaaggtgtcctaacgcaaaaactgggaccttggcgtcggccggtggcctacctgtccaaaaagctagacccagtagcagctgggtggcccccttgcctacggatggtagcagccattgccgtactgacaaaggatgcaggcaagctaaccatgggacagccactagtcattctggccccccatgcagtagaggcactagtcaaacaaccccccgaccgctggctttccaacgcccggatgactcactatcaggccttgcttttggacacggaccgggtccagttcggaccggtggtagccctgaacccggctacgctgctcccactgcctgaggaagggctgcaacacaactgccttgatatcctggccgaagcccacggaacccgacccgacctaacggaccagccgctcccagacgccgaccacacctggtacacggatggaagcagtctcttacaagagggacagcgtaaggcgggagctgcggtgaccaccgagaccgaggtaatctgggctaaagccctgccagccgggacatccgctcagcgggctgaactgatagcactcacccaggccctaaagatggcagaaggtaagaagctaaatgtttatactgatagccgttatgcttttgctactgcccatatccatggagaaatatacagaaggcgtgggttgctcacatcagaaggcaaagagatcaaaaataaagacgagatcttggccctactaaaagccctctttctgcccaaaagacttagcataatccattgtccaggacatcaaaagggacacagcgccgaggctagaggcaaccggatggctgaccaagcggcccgaaaggcagccatcacagagactccagacacctctaccctcctcatagaaaattcatcaccctacacctcagaacattttcattacacagtgactgatataaaggacctaaccaagttgggggccatttatgataaaacaaagaagtattgggtctaccaaggaaaacctgtgatgcctgaccagtttacttttgaattattagactttcttcatcagctgactcacctcagcttctcaaaaatgaaggctctcctagagagaagccacagtccctactacatgctgaaccgggatcgaacactcaaaaatatcactgagacctgcaaagcttgtgcacaagtcaacgccagcaagtctgccgttaaacagggaactagggtccgcgggcatcggcccggcactcattgggagatcgatttcaccgagataaagcccggattgtatggctataaatatcttctagtttttatagataccttttctggctggatagaagccttcccaaccaagaaagaaaccgccaaggtcgtaaccaagaagctactagaggagatcttccccaggttcggcatgcctcaggtattgggaactgacaatgggcctgccttcgtctccaaggtgagtcagacagtggccgatctgttggggattgattggaaattacattgtgcatacagaccccaaagctcaggccaggtagaaagaatgaatagaaccatcaaggagactttaactaaattaacgcttgcaactggctctagagactgggtgctcctactccccttagccctgtaccgagcccgcaacacgccgggcccccatggcctcaccccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgacatggcgcgttcaacgctctcaaaaccccctcaagataagattaacccgtggaagcccttaatagtcatgggagtcctgttaggagtagggatggcagagagcccccatcaggtctttaatgtaacctggagagtcaccaacctgatgactgggcgtaccgccaatgccacctccctcctgggaactgtacaagatgccttcccaaaattatattttgatctatgtgatctggtcggagaggagtgggacccttcagaccaggaaccgtatgtcgggtatggctgcaagtaccccgcagggagacagcggacccggacttttgacttttacgtgtgccctgggcataccgtaaagtcggggtgtgggggaccaggagagggctactgtggtaaatgggggtgtgaaaccaccggacaggcttactggaagcccacatcatcgtgggacctaatctcccttaagcgcggtaacaccccctgggacacgggatgctctaaagttgcctgtggcccctgctacgacctctccaaagtatccaattccttccaaggggctactcgagggggcagatgcaaccctctagtcctagaattcactgatgcaggaaaaaaggctaactgggacgggcccaaatcgtggggactgagactgtaccggacaggaacagatcctattaccatgttctccctgacccggcaggtccttaatgtgggaccccgagtccccatagggcccaacccagtattacccgaccaaagactcccttcctcaccaatagagattgtaccggctccacagccacctagccccctcaataccagttaccccccttccactaccagtacaccctcaacctcccctacaagtccaagtgtcccacagccacccccaggaactggagatagactactagctctagtcaaaggagcctatcaggcgcttaacctcaccaatcccgacaagacccaagaatgttggctgtgcttagtgtcgggacctccttattacgaaggagtagcggtcgtgggcacttataccaatcattccaccgctccggccaactgtacggccacttcccaacataagcttaccctatctgaagtgacaggacagggcctatgcatgggggcagtacctaaaactcaccaggccttatgtaacaccacccaaagcgccggctcaggatcctactaccttgcagcacccgccggaacaatgtgggcttgcagcactggattgactccctgcttgtccaccacggtgctcaatctaaccacagattattgtgtattagttgaactctggcccagagtaatttaccactcccccgattatatgtatggtcagcttgaacagcgtaccaaatataaaagagagccagtatcattgaccctggcccttctactaggaggattaaccatgggagggattgcagctggaatagggacggggaccactgccttaattaaaacccagcagtttgagcagcttcatgccgctatccagacagacctcaacgaagtcgaaaagtcaattaccaacctagaaaagtcactgacctcgttgtctgaagtagtcctacagaaccgcagaggcctagatttgctattcctaaaggagggaggtctctgcgcagccctaaaagaagaatgttgtttttatgcagaccacacggggctagtgagagacagcatggccaaattaagagaaaggcttaatcagagacaaaaactatttgagacaggccaaggatggttcgaagggctgtttaatagatccccctggtttaccaccttaatctccaccatcatgggacctctaatagtactcttactgatcttactctttggaccttgcattctcaatcgattggtccaatttgttaaagacaggatctcagtggtccaggctctggttttgactcagcaatatcaccagctaaaacccatagagtacgagccatgaacgcgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaancgtctaggccccccgaaccacggggacgtggttttcctttgaaaaacacgattataaatggtgaccggcggcatggcctccaagtgggatcaaaagggcatggatatcgcttacgaggaggccctgctgggctacaaggagggcggcgtgcctatcggcggctgtctgatcaacaacaaggacggcagtgtgctgggcaggggccacaacatgaggttccagaagggctccgccaccctgcacggcgagatctccaccctggagaactgtggcaggctggagggcaaggtgtacaaggacaccaccctgtacaccaccctgtccccttgtgacatgtgtaccggcgctatcatcatgtacggcatccctaggtgtgtgatcggcgagaacgtgaacttcaagtccaagggcgagaagtacctgcaaaccaggggccacgaggtggtggttgttgacgatgagaggtgtaagaagctgatgaagcagttcatcgacgagaggcctcaggactggttcgaggatatcggcgagtaagcggccgcagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcattacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattccat

SEQ ID NO: 43 - DNA, 13 bp, Artificial Sequence
  Core minipromoter domain
ccccgttgcccgg

SEQ ID NO: 44 - DNA, 10 bp, Artificial Sequence
  CArG enhancer motif
ccatataagg

SEQ ID NO: 45 - DNA, 10 bp, Artificial Sequence
  NFkB1 Enhancer Motif
ggaaatcccc

SEQ ID NO: 46 - DNA, 11 bp, Artificial Sequence
  NFkB2 Enhancer Motif
ggaaagtcccc

SEQ ID NO: 47 - DNA, 22 bp, Artificial Sequence
  Env2 forward primer
accctcaacctcccctacaagt

SEQ ID NO: 48 - DNA, 20 bp, Artificial Sequence
  Env2 Reverse Primer
gttaagcgcctgataggctc

SEQ ID NO: 49 - DNA, 26 bp, Artificial Sequence
  Env2 Probe Sequence
agccacccccaggaactggagataga

SEQ ID NO: 50 - DNA, 23 bp, Artificial Sequence
  yCD2 Forward Primer
atcatcatgtacggcatccctag

SEQ ID NO: 51 - DNA, 24 bp, Artificial Sequence
  yCD2 Reverse Primer
tgaactgcttcatcagcttcttac

SEQ ID NO: 52 - DNA, 25 bp, Artificial Sequence
  yCD2 Probe Sequence
tcatcgtcaacaaccaccacctcgt

SEQ ID NO: 53 - DNA, 23 bp, Artificial Sequence
  Forward Primer
ctgatcttactctttggaccttg

SEQ ID NO: 54 - DNA, 24 bp, Artificial Sequence
  Reverse Primer
cccctttttctggagactaaataa

(19) United States
(12) Patent Application Publication
(10) Pub. No.: US 2016/0222412 A1
(43) Pub. Date: Aug. 4, 2016

(54) RECOMBINANT VECTOR WITH STABILIZING A-LOOP

(71) Applicant: Tocagen Inc., San Diego, CA (US)

(72) Inventors: Amy H. Lin, San Diego, CA (US); Harry E. Gruber, Rancho Santa Fe, CA (US); Carlos Ibanez, San Diego, CA (US); Douglas J. Jolly, Encinitas, CA (US)

(21) Appl. No.: 15/016,201

(22) Filed: Feb. 4, 2016

Related U.S. Application Data
(63) Continuation-in-part of application No. PCT/US2014/049831, filed on Aug. 5, 2014.
(60) Provisional application No. 61/862,433, filed on Aug. 5, 2013.
(60) Provisional application No. 62/205,683, filed on Aug. 15, 2015.

(51) Int. Cl. C12N 15/86

(52) U.S. Cl. CPC C12N 15/86; C12N 2740/10043; C12N 2830/85

(57) ABSTRACT

The disclosure describes replication competent retroviral vectors (RCR) for gene therapy and gene delivery. The RCR includes an IRES sequence having 5-6A's in A-bulge of the bifurcation region.

### CROSS REFERENCE TO RELATED APPLICATIONS

This application is a continuation-in-part application of International Application No. PCT/US2014/049831, filed Aug. 5, 2014, which claims priority to U.S. Provisional Application Ser. No. 61/862,433, filed Aug. 5, 2013. This application also claims priority to U.S. Provisional Application Ser. No. 62/205,683, filed Aug. 15, 2015, the disclosures of which are incorporated herein by reference.

### TECHNICAL FIELD

This disclosure relates to optimized internal ribosome entry sites (IRES), compositions containing such optimized IRESs including vectors. More particularly, the disclosure relates to replication competent retroviral vectors for treating cell proliferative disorders. The disclosure further relates to the use of such replication competent retroviral vectors for delivery and expression of heterologous nucleic acids.

### BACKGROUND

Effective methods of delivering genes and heterologous nucleic acids to cells and subjects has been a goal researchers for scientific development and for possible treatments of diseases and disorders.

### INCORPORATION OF SEQUENCE LISTING

The present application is filed with a Sequence Listing in electronic format. The Sequence Listing is provided as a file entitled 00014-019US1Sequence ST25.txt created on Feb. 4, 2016, which is 205 Kb in size. The information in the electronic format of the sequence listing is incorporated herein by reference in its entirety.

### SUMMARY

The disclosure provides a cassette comprising an internal ribosome entry site (IRES) consisting of 5-6A's in the A-bulge in the bifurcation region of the IRES, wherein the IRES is operably linked to a heterologous polynucleotide. An IRES of the disclosure having 5As has the advantage over IRESes with 6 or 7As in the bifurcation loop for expressing a protein from the heterologous polynucleotide sequence at levels essentially equivalent to 7As and within 60% of an equivalent IRES with 6A's, while maintaining stability. An IRES of the disclosure having 6As has the advantage over IRESes with fewer than 5 As or more than 7As in the bifurcation loop having improved expression of a protein from the heterologous polynucleotide sequence. The disclosure also provides viral vectors comprising an IRES wth 5A or 6As to express a protein. The disclosure further provides IRESes with 5 or 6A's incorporated into a replication competent vector or an RNA-based vector. In a further embodiment the dislcure provides a recombinant replication competent retrovirus comprising: a retroviral GAG protein; a retroviral POL protein; a retroviral envelope; a retroviral polynucleotide comprising Long-Terminal Repeat (LTR) sequences at the 3′ end of the retroviral polynucleotide sequence, a promoter sequence at the 5′ end of the retroviral polynucleotide, said promoter being suitable for expression in a mammalian cell, a gag nucleic acid domain, a pol nucleic acid domain and an env nucleic acid domain; a cassette comprising an internal ribosome entry site (IRES) consisting of 5 or 6A's in the A-bulge in the bifurcation region of the IRES, wherein the IRES is operably linked to a heterologous polynucleotide, wherein the cassette is positioned 5′ to the 3′ LTR and 3′ to the env nucleic acid domain encoding the retroviral envelope; and cis-acting sequences necessary for reverse transcription, packaging and integration in a target cell, wherein the RCR maintains higher replication competency or expression levels compared to a vector comprising less than 5As or greater than 7A's in the A-Bulge. In one embodiment, the virus infects a target cell multiple times resulting in an average number of copies/diploid genome of 5 or greater. In another embodiment of any of the foregoing, the retroviral polynucleotide sequence is derived from a virus selected from the group consisting of murine leukemia virus (MLV), Moloney murine leukemia virus (MoMLV), Feline leukemia virus (FeLV), Baboon endogenous retrovirus (BEV), porcine endogenous virus (PERV), the cat derived retrovirus RD114, squirrel monkey retrovirus, Xenotropic murine leukemia virus-related virus(XMRV), avian reticuloendotheliosis virus(REV), or Gibbon ape leukemia virus (GALV). In another embodiment of any of the foregoing, the retroviral envelope is an amphotropic MLV envelope. In another embodiment of any of the foregoing, the retrovirus is a gammaretrovirus. In another embodiment of any of the foregoing, the target cell is a cell having a cell proliferative disorder. In another embodiment of any of the foregoing, target cell is a neoplastic cell. In another embodiment of any of the foregoing, the cell proliferative disorder is selected from the group consisting of lung cancer, colon-rectum cancer, breast cancer, prostate cancer, urinary tract cancer, uterine cancer, brain cancer, head and neck cancer, pancreatic cancer, melanoma, stomach cancer and ovarian cancer, rheumatoid arthritis or other autoimmune disease. In another embodiment of any of the foregoing, the promoter sequence is associated with a growth regulatory gene. In another embodiment of any of the foregoing, the promoter sequence comprises a tissue-specific promoter sequence. In another embodiment of any of the foregoing, the tissue-specific promoter sequence comprises at least one androgen response element (ARE). In another embodiment of any of the foregoing, the promoter comprises a CMV promoter having a sequence as set forth in SEQ ID NO:19, 20, 22 or 42 from nucleotide 1 to about nucleotide 582 and may include modification to one or more nucleic acid bases and which is capable of directing and initiating transcription In another embodiment of any of the foregoing, the promoter comprises a CMV-R-U5 domain polynucleotide. In another embodiment of any of the foregoing, the CMV-R-U5 domain comprises the immediately early promoter from human cytomegalovirus linked to an MLV R-U5 region. In another embodiment of any of the foregoing, the CMV-R-U5 domain polynucleotide comprises a sequence as set forth in SEQ ID NO: 19, 20, 22 or 42 from about nucleotide 1 to about nucleotide 1202 or sequences that are at least 95% identical to a sequence as set forth in SEQ ID NO:19, 20, 22 or 42, wherein the polynucleotide promotes transcription of a nucleic acid molecule operably linked thereto. In another embodiment of any of the foregoing, the gag polynucleotide is derived from a gammaretrovirus. In another embodiment of any of the foregoing, the gag nucleic acid domain comprises a sequence from about nucleotide number 1203 to about nucleotide 2819 of SEQ ID NO: 19, 20, 22 or 42 or a sequence having at least 95%, 98%, 99% or 99.8% identity thereto. In another embodiment of any of the foregoing, the pol domain of the polynucleotide is derived from a gammaretrovirus. In another embodiment of any of the foregoing, the pol domain comprises a sequence from about nucleotide number 2820 to about nucleotide 6358 of SEQ ID NO: 19, 20, 22 or 42 or a sequence having at least 95%, 98%, 99% or 99.9% identity thereto. In another embodiment of any of the foregoing, the env domain comprises a sequence from about nucleotide number 6359 to about nucleotide 8323 of SEQ ID NO: 19, 20, 22 or 42 or a sequence having at least 95%, 98%, 99% or 99.8% identity thereto. In another embodiment of any of the foregoing,the IRES consists of a sequence that is at least 90% identical to the sequence set forth in SEQ ID NO:41 comprising 5 or 6As in the A-bulge. In another embodiment of any of the foregoing, the retroviral polynucleotide sequence comprises (i) the sequence set forth in SEQ ID NO:42 or (ii) the sequence as set forth in SEQ ID NO:42, wherein T is U. In another embodiment of any of the foregoing, the heterologous nucleic acid comprises a polynucleotide having a sequence as set forth in SEQ ID NO:3, 5, 11, 13, 15 or 17. In another embodiment of any of the foregoing, the heterologous nucleic acid encodes a polypeptide comprising a sequence as set forth in SEQ ID NO:4. In another embodiment of any of the foregoing, the heterologous nucleic acid is human codon optimized and encodes a polypeptide as set forth in SEQ ID NO:4. In another embodiment the heterologous gene is a humanized thymidine kinase. In another embodiment of any of the foregoing, the heterologous nucleic acid comprises a sequence as set forth in SEQ ID NO: 19 or 22 from about nucleotide number 8877 to about 9353. In another embodiment of any of the foregoing, the 3′ LTR is derived from a gammaretrovirus. In another embodiment of any of the foregoing, the 3′ LTR comprises a U3-R-U5 domain. In another embodiment of any of the foregoing,the 3′ LTR comprises a sequence as set forth in SEQ ID NO: 19 or 22 from about nucleotide 9405 to about 9998 or a sequence that is at least 95%, 98% or 99.5% identical thereto. In another embodiment of any of the foregoing, the heterologous nucleic acid sequence encodes a biological response modifier or an immunopotentiating cytokine. In another embodiment of any of the foregoing, the immunopotentiating cytokine is selected from the group consisting of interleukins 1 through 15, interferon, tumor necrosis factor (TNF), and granulocyte-macrophage-colony stimulating factor (GM-CSF). In another embodiment of any of the foregoing, the immunopotentiating cytokine is interferon gamma. In another embodiment of any of the foregoing, the heterologous nucleic acid encodes a polypeptide that converts a nontoxic prodrug in to a toxic drug. In another embodiment of any of the foregoing,the polypeptide that converts a nontoxic prodrug in to a toxic drug is thymidine kinase, purine nucleoside phosphorylase (PNP), or cytosine deaminase. In another embodiment of any of the foregoing, the heterologous nucleic acid sequence encodes a receptor domain, an antibody, or antibody fragment. In another embodiment of any of the foregoing, the heterologous nucleic acid sequence comprises an inhibitory polynucleotide. In another embodiment of any of the foregoing, the inhibitory polynucleotide comprises an miRNA, RNAi or siRNA sequence.

The disclosure also provides a recombinant retroviral polynucleotide genome for producing a replication competent retrovirus as described above.

The disclosure also provides a method of treating a cell proliferative disorder comprising contacting the subject with a recombinant replication competent retrovirus of the disclosure under conditions such that the cytosine deaminase polynucleotide is expressed and contacting the subject with 5-fluorocytosine. In another embodiment, the cell proliferative disorder is glioblastoma multiforme. In another embodiment of any of the foregoing,the cell proliferative disorder is selected from the group consisting of lung cancer, colon-rectum cancer, breast cancer, prostate cancer, urinary tract cancer, uterine cancer, brain cancer, head and neck cancer, pancreatic cancer, melanoma, stomach cancer and ovarian cancer.

The disclosure also provides a vector that expresses a heterologous gene in a mammalian cell from an ECMV IRES with 5As in the A bulge in the J-K bifurcation region. In another embodiment, the vector is a viral vector. In another embodiment of any of the foregoing, the vector is a retroviral replicating vector. In another embodiment of any of the foregoing, the vector is a retroviral replicating vector derived from a gamma-retrovirus. In another embodiment of any of the foregoing, the gamma-retrovirus is derived from one of Murine Leukemia Virus, Baboon Endogenous Virus, Gibbon Ape Leukemia virus, Feline leukemia virus. In another embodiment of any of the foregoing, the heterologous gene is a gene with a therapeutic activity in mammals In another embodiment of any of the foregoing, the therapeutic activity is an anticancer activity. In another embodiment of any of the foregoing, the heterologous gene is a prodrug activating gene. In another embodiment of any of the foregoing, the vector can express a heterologous gene in a mammalian cell from an ECMV IRES in the absence of the protein PTB-1.

The disclosure also provides a method of treating cancer, by administering a vector as described above.

The disclosure also provides a recombinant replication competent retrovirus comprising: a retroviral GAG protein; a retroviral POL protein; a retroviral envelope; a retroviral polynucleotide comprising Long-Terminal Repeat (LTR) sequences at the 3′ end of the retroviral polynucleotide sequence, a promoter sequence at the 5′ end of the retroviral polynucleotide, said promoter being suitable for expression in a mammalian cell, a gag nucleic acid domain, a pol nucleic acid domain and an env nucleic acid domain; a cassette comprising a minimal internal ribosome entry site (IRES), wherein the minimal IRES is operably linked to a heterologous polynucleotide, and may further comprise (i) a polIII promoter linked to an miRNA or (ii) a mini-promoter operably linked to a heterologous polynucleotide that is proceeds or follows (i), wherein the cassette is positioned 5′ to the 3′ LTR and 3′ to the env nucleic acid domain encoding the retroviral envelope; and cis-acting sequences necessary for reverse transcription, packaging and integration in a target cell. In one embodiment, the minimal IRES consists of a sequence from about base 123 to 544 of SEQ ID NO:41. In another embodiment of any of the foregoing, the minimum IRES consists of a sequence from about base 183 to 544 of SEQ ID NO:41. In another embodiment of any of the foregoing, the IRES has 5As in the A bulge. In another embodiment of any of the foregoing, the virus infects a target cell multiple times resulting in an average number of copies/diploid genome of 5 or greater. In another embodiment of any of the foregoing, the retroviral polynucleotide sequence is derived from a virus selected from the group consisting of murine leukemia virus (MLV), Moloney murine leukemia virus (MoMLV), Feline leukemia virus (FeLV), Baboon endogenous retrovirus (BEV), porcine endogenous virus (PERV), the cat derived retrovirus RD114, squirrel monkey retrovirus, Xenotropic murine leukemia virus-related virus(XMRV), avian reticuloendotheliosis virus(REV), or Gibbon ape leukemia virus (GALV). In another embodiment of any of the foregoing, the retroviral envelope is an amphotropic MLV envelope. In another embodiment of any of the foregoing, the retrovirus is a gammaretrovirus. In another embodiment of any of the foregoing, the target cell is a cell having a cell proliferative disorder. In another embodiment of any of the foregoing, the target cell is a neoplastic cell. In another embodiment of any of the foregoing, the cell proliferative disorder is selected from the group consisting of lung cancer, colon-rectum cancer, breast cancer, prostate cancer, urinary tract cancer, uterine cancer, brain cancer, head and neck cancer, pancreatic cancer, melanoma, stomach cancer and ovarian cancer, rheumatoid arthritis or other autoimmune disease. In another embodiment of any of the foregoing, the promoter sequence is associated with a growth regulatory gene. In another embodiment of any of the foregoing, the promoter sequence comprises a tissue-specific promoter sequence. In another embodiment of any of the foregoing, the tissue-specific promoter sequence comprises at least one androgen response element (ARE). In another embodiment of any of the foregoing, the promoter comprises a CMV promoter having a sequence as set forth in SEQ ID NO:19, 20, 22 or 42 from nucleotide 1 to about nucleotide 582 and may include modification to one or more nucleic acid bases and which is capable of directing and initiating transcription. In another embodiment of any of the foregoing, the promoter comprises a CMV-R-U5 domain polynucleotide. In another embodiment of any of the foregoing, the CMV-R-U5 domain comprises the immediately early promoter from human cytomegalovirus linked to an MLV R-U5 region. In another embodiment of any of the foregoing, the CMV-R-U5 domain polynucleotide comprises a sequence as set forth in SEQ ID NO: 19, 20, 22 or 42 from about nucleotide 1 to about nucleotide 1202 or sequences that are at least 95% identical to a sequence as set forth in SEQ ID NO:19, 20, 22 or 42, wherein the polynucleotide promotes transcription of a nucleic acid molecule operably linked thereto. In another embodiment of any of the foregoing, the gag polynucleotide is derived from a gammaretrovirus. In another embodiment of any of the foregoing, the gag nucleic acid domain comprises a sequence from about nucleotide number 1203 to about nucleotide 2819 of SEQ ID NO: 19, 20, 22 or 42 or a sequence having at least 95%, 98%, 99% or 99.8% identity thereto. In another embodiment of any of the foregoing, the pol domain of the polynucleotide is derived from a gammaretrovirus. In another embodiment of any of the foregoing, the pol domain comprises a sequence from about nucleotide number 2820 to about nucleotide 6358 of SEQ ID NO: 19, 20, 22 or 42 or a sequence having at least 95%, 98%, 99% or 99.9% identity thereto. In another embodiment of any of the foregoing, the env domain comprises a sequence from about nucleotide number 6359 to about nucleotide 8323 of SEQ ID NO: 19, 20, 22 or 42 or a sequence having at least 95%, 98%, 99% or 99.8% identity thereto. In another embodiment of any of the foregoing, the heterologous nucleic acid comprises a polynucleotide having a sequence as set forth in SEQ ID NO:3, 5, 11, 13, 15 or 17. In another embodiment of any of the foregoing, the heterologous nucleic acid encodes a polypeptide comprising a sequence as set forth in SEQ ID NO:4. In another embodiment of any of the foregoing, the heterologous nucleic acid is human codon optimized and encodes a polypeptide as set forth in SEQ ID NO:4. In another embodiment of any of the foregoing, the heterologous nucleic acid comprises a sequence as set forth in SEQ ID NO: 19 or 22 from about nucleotide number 8877 to about 9353. In another embodiment of any of the foregoing, the 3′ LTR is derived from a gammaretrovirus. In another embodiment of any of the foregoing, the 3′ LTR comprises a U3-R-U5 domain. In another embodiment of any of the foregoing, the 3′ LTR comprises a sequence as set forth in SEQ ID NO: 19 or 22 from about nucleotide 9405 to about 9998 or a sequence that is at least 95%, 98% or 99.5% identical thereto. In another embodiment of any of the foregoing, the heterologous nucleic acid sequence encodes a biological response modifier or an immunopotentiating cytokine. In another embodiment of any of the foregoing, the immunopotentiating cytokine is selected from the group consisting of interleukins 1 through 15, interferon, tumor necrosis factor (TNF), and granulocyte-macrophage-colony stimulating factor (GM-CSF). In another embodiment of any of the foregoing, the immunopotentiating cytokine is interferon gamma. In another embodiment of any of the foregoing, the heterologous nucleic acid encodes a polypeptide that converts a nontoxic prodrug in to a toxic drug. In another embodiment of any of the foregoing,the polypeptide that converts a nontoxic prodrug in to a toxic drug is thymidine kinase, purine nucleoside phosphorylase (PNP), or cytosine deaminase. In another embodiment of any of the foregoing, the heterologous nucleic acid sequence encodes a receptor domain, an antibody, or antibody fragment. In another embodiment of any of the foregoing, the heterologous nucleic acid sequence comprises an inhibitory polynucleotide. In another embodiment of any of the foregoing, the inhibitory polynucleotide comprises an miRNA, RNAi or siRNA sequence.

The details of one or more embodiments of the disclosure are set forth in the accompanying drawings and the description below. Other features, objects, and advantages will be apparent from the description and drawings, and from the claims.

### BRIEF DESCRIPTION OF DRAWINGS

FIG. 1A-C shows replicating retroviral vectors containing IRES with various numbers of A's in the A bulge and their titers. (A) Predicted secondary structure of the EMCV internal ribosomal entry site (SEQ ID NO:41). The sequences start from position 680. Circled capital letter J, K, L and M indicate defined region in the IRES. Arrow indicates the bifurcation loop in the J-K region. AUG8, AUG9, AUG10 and AUG11 are underlined. (B) Diagram of the A bulge in the J-K bifurcation region in EMCV IRES incorporated into RRV expressing yCD2 or GFP. The native ATG8 (AUG in RNA) and ATG9 are underlined; enlarged and underlined sequence represents the A bulge in the J-K bifurcation region; lower case letters indicate the 5′ sequences in the polypyrimidine tract in the 3′ IRES; (C) Viral titer of RRV containing various numbers of As in the A bulge produced by infected HT1080 cells.

FIG. 2A-D shows cellular viral derived RNA and protein expression by RRV with various numbers of A's in the A bulge. (A) Schematic diagram of cellular viral RNA isoforms. Env2 primers and probe, and yCD2 primers and probe recognize both unspliced and spliced viral RNA in the env and the yCD2 region, respectively, were used to measure the level of cellular viral RNA by qRT-PCR. Filled triangles: env2 primer and probe set; open triangles: yCD2 primer and probe set. (B) Immunoblot of yCD2 and GAPDH protein. Twenty micrograms of cell lysate were loaded to each lane and equivalent loading and blotting efficiency controlled for by detection of the ubiquitous marker GAPDH. PC, positive control; NC, negative control. Graph represents the RNA and protein expression levels relative to the yCD2-6A vector. (C) RNA and GFP expression levels relative to the GFP-6A vector. The percentage GFP positive cells were determined by flow cytometry using proper gating to exclude GFP-negative cells. GFP protein expression levels were quantified by using mean fluorescent intensity (D) Proviral vector copy number of infected U87-MG cells (MOI of 0.01) by qPCR. Genomic DNA is isolated day 14 post infection at which the vector with 7A is expected to be maximally infected. The data show that there is no significant difference in vector copy of number of maximally infected U87-MG cells. This is consistent with viral production data in which no significant effect on viral titer is observed among the variants.

FIG. 3 shows a vector sequence (SEQ ID NO:22) with an A-bulge underlined and bolded.

FIG. 4A-B shows vector stability data. (A) Vectors stability in infected U87-MG cells (MOI of 0.01) by end-point PCR. Genomic DNA is isolated day 14 post infection and the IRES-yCD2 region is amplified using the primer set spanning the 3′ of the env and 3′UTR region (Perez et al., 2012). (B) Assessment of vector stability by serial infection. Approximately 10⁵ naive U87-MG cells were initially infected with the viral vectors at a MOI of 0.1 and grown for 1 week to complete a single cycle of infection. 100 μL of the 2 ml of viral supernatant from fully infected cells is used to infect naive cells and repeated up to 12 cycles. Vector stability of the IRES-yCD2 region is assessed by PCR amplification of the integrated provirus from the infected cells. The expected PCR product size is approximately 1.2 kb. The appearance of any bands smaller than 1.2 kb indicate deletion in the IRES-yCD2 region.

FIG. 5 shows a diagram of a construct of the disclosure designed with minimal IRESs (the sequence below the schematic corresponds to SEQ ID NO:41 from base 123-139; and 183 to 198).

FIG. 6 shows yCD2 expression from transiently transfected 293T cells. GAPDH detection was included as a loading control. Positive control (+) is lysate from U87-MG cells infected with RRV-yCD2 vector.

FIG. 7 shows Replication kinetics of RRV-yCD2 variants in U87-MG cells. Replication kinetics of RRV-yCD2 carrying various length of As in the A bulge was measured by the average vector copy number in infected U87-MG cells (MOI of 0.01) at indicated time points during the course of infection.

FIG. 8A-F shows RNA and protein expression from RRV with various numbers of As in the A bulge. (A) Schematic diagram of cellular viral RNA isoforms. The env2 and yCD2 primer-probe sets, which recognize both unspliced and spliced viral RNA in the env and the yCD2 region, respectively, were used to measure the level of cellular viral RNA by qRT-PCR. Filled triangles: env2 primer and probe set; open triangles: yCD2 primer and probe set. (B) Cellular viral RNA expression levels relative to yCD2-6A using the yCD2 and env2 primer sets. (C) Immunoblot of yCD2 and GAPDH protein. Twenty micrograms of cell lysate were loaded to each lane and equivalent loading and blotting efficiency controlled for by detection of the ubiquitous marker GAPDH. NC, negative control. (D) Graph represents the RNA and protein expression levels relative to the yCD2-6A vector. (E) Cell-based enzymatic activity of yCD2 in infected U87-MG cells was measured by HPLC to detect the amount of 5-FU. The 5-FU peak area of each vector is plotted relative to yCD2-6A vector which is set to 1. (F) RNA and GFP expression levels relative to the GFP-6A vector. The percentage GFP positive cells were determined by flow cytometry using proper gating to exclude GFP-negative cells. GFP protein expression levels were quantified by using mean fluorescence intensity (MFI).

FIG. 9A-B shows vector stability of RRV-IRES-yCD2 variants in infected U87-MG cells. (A) Stability of proviral DNA of IRES-yCD2 cassette in RRV-IRES-yCD2 variants from one round infection showed no detection of deletion mutants. (B) Stability of proviral DNA of IRES-yCD2 transgene in RRV-yCD2-6A and RRV-yCD2-7A over 12 cycles of serial infection. DNA molecular marker (1 kb plus marker, Invitrogen) is included in the first lane of each gel. The numbers above each lane indicate the number of infection cycle for each vector. NTC, no template control. Asterisk indicates a deletion of the IRES-yCD2 cassette.

FIG. 10A-B shows protein expression level of yCD2 in RRV-IRES-yCD2 variants decreases due to expansion of the oligo A length in bulge A. Protein expression of yCD2 in RRV-IRES-yCD2 variants were evaluated at infection cycle 7 (A) and 10 (B) to correlate with expansion of the oligo A length in bulge A observed in sequencing results. Vector stability analyzed by PCR is included to detect deletion of IRES-yCD2 cassette and noted as an additional factor in some variants contributing to the reduction of yCD2 protein expression. NTC, no template control. +, positive control using RRV-IRES-6A plasmid DNA as a template in PCR. Asterisk indicates a deletion of the IRES-yCD2 cassette.

### DETAILED DESCRIPTION

As used herein and in the appended claims, the singular forms “a,” “and,” and “the” include plural referents unless the context clearly dictates otherwise. Thus, for example, reference to “a cell” includes a plurality of such cells and reference to “the agent” includes reference to one or more agents known to those skilled in the art, and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

General texts which describe molecular biological techniques useful herein, including the use of vectors, promoters and many other relevant topics, include Berger and Kimmel, Guide to Molecular Cloning Techniques, Methods in Enzymology Volume 152, (Academic Press, Inc., San Diego, Calif.) (“Berger”); Sambrook et al., Molecular Cloning—A Laboratory Manual, 2d ed., Vol. 1-3, Cold Spring Harbor Laboratory, Cold Spring Harbor, N.Y., 1989 (“Sambrook”) and Current Protocols in Molecular Biology, F. M. Ausubel et al., eds., Current Protocols, a joint venture between Greene Publishing Associates, Inc. and John Wiley & Sons, Inc., (supplemented through 1999) (“Ausubel”). Examples of protocols sufficient to direct persons of skill through in vitro amplification methods, including the polymerase chain reaction (PCR), the ligase chain reaction (LCR), Qβ-replicase amplification and other RNA polymerase mediated techniques (e.g., NASBA), e.g., for the production of the homologous nucleic acids of the disclosure are found in Berger, Sambrook, and Ausubel, as well as in Mullis et al. (1987) U.S. Pat. No. 4,683,202; Innis et al., eds. (1990) PCR Protocols: A Guide to Methods and Applications (Academic Press Inc. San Diego, Calif.) (“Innis”); Arnheim & Levinson (Oct. 1, 1990) C&EN 36-47; The Journal Of NIH Research (1991) 3: 81-94; Kwoh et al. (1989) Proc. Natl. Acad. Sci. USA 86: 1173; Guatelli et al. (1990) Proc. Nat'l. Acad. Sci. USA 87: 1874; Lomell et al. (1989) J. Clin. Chem 35: 1826; Landegren et al. (1988) Science 241: 1077-1080; Van Brunt (1990) Biotechnology 8: 291-294; Wu and Wallace (1989) Gene 4:560; Barringer et al. (1990) Gene 89:117; and Sooknanan and Malek (1995) Biotechnology 13: 563-564. Improved methods for cloning in vitro amplified nucleic acids are described in Wallace et al., U.S. Pat. No. 5,426,039. Improved methods for amplifying large nucleic acids by PCR are summarized in Cheng et al. (1994) Nature 369: 684-685 and the references cited therein, in which PCR amplicons of up to 40 kb are generated. One of skill will appreciate that essentially any RNA can be converted into a double stranded DNA suitable for restriction digestion, PCR expansion and sequencing using reverse transcriptase and a polymerase. See, e.g., Ausubel, Sambrook and Berger, all supra.

The publications discussed throughout the text are provided solely for their disclosure prior to the filing date of the present application. Nothing herein is to be construed as an admission that the inventors are not entitled to antedate such disclosure by virtue of prior disclosure.

The disclosure provides methods and compositions useful for gene or protein delivery to a cell or subject. Such methods and compositions can be used to treat various diseases and disorders in a subject including cancer and other cell proliferative diseases and disorders. In one embodiment, the disclosure provides optimized IRESs. Such optimized IRESs can be used in various vectors to facilitate protein expression. In another aspect, the disclosure provides replication competent retroviral vectors for gene delivery. The disclosure demonstrates that commonly used IRESs containing 7A's in the A-bulge in the J-K bifurcation region are not optimal and thus the disclosure provides an IRES with an optimal A bulge sequence having improved polypeptide expression and/or stability compared to IRESs with fewer (less than 4) or more (7-8) As.

An internal ribosome entry sites (“IRES”) refers to a segment of nucleic acid that promotes the entry or retention of a ribosome during translation of a coding sequence usually 3′ to the IRES. In some embodiments the IRES may comprise a splice acceptor/donor site, however, preferred IRESs lack a splice acceptor/donor site. Normally, the entry of ribosomes into messenger RNA takes place via the cap located at the 5′ end of all eukaryotic mRNAs. However, there are exceptions to this universal rule. The absence of a cap in some viral mRNAs suggests the existence of alternative structures permitting the entry of ribosomes at an internal site of these RNAs. To date, a number of these structures, designated IRES on account of their function, have been identified in the 5′ noncoding region of uncapped viral mRNAs, including, for example, that of picornaviruses such as poliomyelitis virus (Pelletier et al., 1988, Mol. Cell. Biol., 8, 1103-1112) and the EMCV virus (encephalo-myocarditis virus) (Jang et al., J. Virol., 1988, 62, 2636-2643). The disclosure provides the use of an optimized IRES in the context of a vector and more particularly a replication-competent retroviral (RCR) vector.

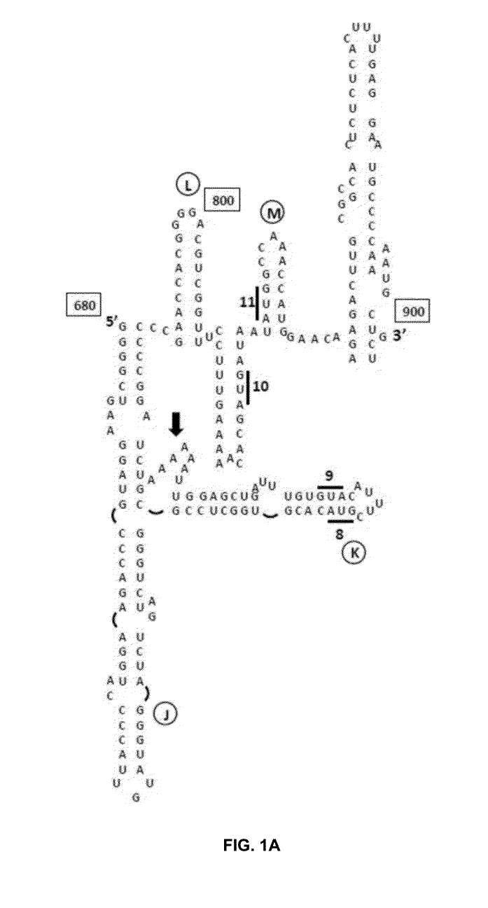

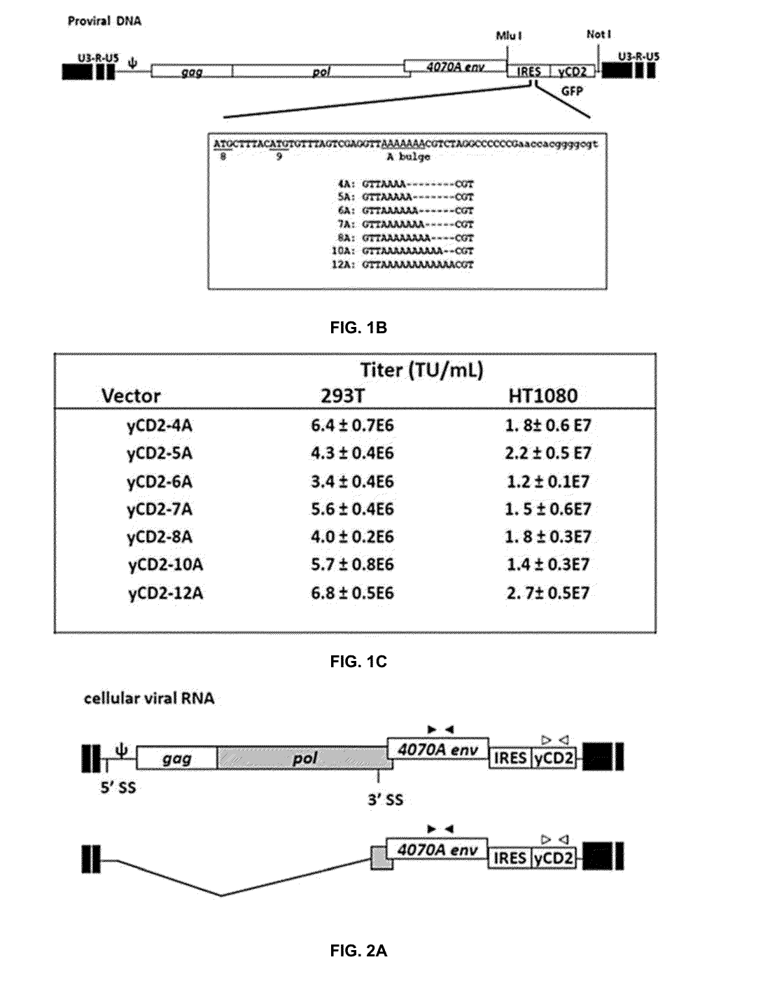

The internal ribosomal entry site (IRES) allows translation of viral RNAs in a cap-independent manner. The IRES from encephalomyocarditis virus (EMCV) has been studied extensively and is widely used in retroviral and other mammalian expression vectors. The proper folding and secondary structure of the IRES dictate its functionality, and sequence changes may or may not affect this. Palmenberg and coworkers showed that, independent of the 5′-IRES region, the J-K elements in the 3′ end of the IRES play a critical role in translation initiation, (FIG. 1A). The sequence of the IRES in various vectors can be found to contain various numbers of polyAs in the A-bulge. For example, Logg et al. (J. Virol. 75:6989-6998, 2001) describes an IRES that carries seven adenosine residues (As) instead of the six As in the A bulge in the bifurcation region (see, e.g., Duke et al., J. Virol. 66:1924-1932, 1992). As described more fully elsewhere herein, the number of A's in the A-bulge affects the expression of an operably associated heterologous sequence and the stability including replication competency of the vectors. For example, the disclosure identifies an optimal number of A's in the A-bulge as peaking at 5-6 A's for expression and stability and expression decreasing slightly the further from the optimal number of A's on either sides. For example, 4 A's is less effective than 5-6 A's and 8 A's is less effective than 5-6 A's.

As used herein an “optimized IRES” refers to an IRES derived from an encephalomyocarditis virus having 5-6As in the A-bulge of the J-K bifurcation region. In one embodiment, the optimized IRES comprise 5As in the A-bulge and has improved stability compared to IRESes with 6 or more As. In another embodiment, the optimized IRES comprises 6As in the A-bulge and has improved expression of a linked coding sequence compared to IRESes with 7As. The optimized IRES can be part of a cassette that comprises a gene or sequence to be expressed (“heterologous polynucleotide” or “gene”). In such instances the optimized IRES is operably linked and upstream of the heterologous polynucleotide sequence and is operably to cause translation of the linked heterologous polynucleotide. The optimized IRES cassette demonstrates increased protein expression from a linked heterologous polynucleotide compared to a non-optimized IRES (e.g., and IRES having 3-4 or 7-8 A's in the A-bulge). An optimized IRES or IRES-cassette can be cloned into any number of vectors for expression of a linked heterologous polynucleotide. For example, vectors that can contain and be used with an optimized IRES or IRES-cassette of the disclosure include plasmids, expression vectors, viral vectors (replication defective and replication competent) and the like.

In one embodiment, the disclosure provides an optimized IRES comprising a sequence selected from the group consisting of: (i) a sequence having 95% identity to SEQ ID NO:41 and having 5-6A's in the J-K bifurcation region; (ii) a truncated IRES comprising a sequence as set forth in SEQ ID NO:41 containing 5-6A's in the bifurcation region and begins anywhere following base pair 1 to about base 183 and continues to 544 of SEQ ID NO:41 (e.g., about 123 to 544 or about 183 to 544 of SEQ ID NO:41) and has improved polypeptide expression compared to a similar IRES with 7As in the bifurcation region; or (iii) a sequence as set forth in SEQ ID NO:41 and (iv) any of the foregoing wherein T can be U (e.g., an RNA version).

A heterologous nucleic acid sequence is operably linked to an optimized IRES consisting of, in one embodiment, 5-6 “As” in the A-bulge region. As used herein, the term “heterologous” nucleic acid sequence or transgene refers to (i) a sequence that does not normally exist in a wild-type retrovirus, (ii) a sequence that originates from a foreign species, (iii) a sequence that is not normally found downstream of an IRES, or (iv) if from the same species, it may be substantially modified from its original form. Alternatively, an unchanged nucleic acid sequence that is not normally expressed in a cell is a heterologous nucleic acid sequence.

In one embodiment, the disclosure provides a vector comprising an optimized IRES in a cassette comprising an A-bulge in the J-K bifurcation region consisting of 5-6As operably linked to a polynucleotide sequence to be expressed. As described in more detail below, an A-bulge consisting of 5-6A's unexpectedly provides superior vector stability and/or protein expression compared to similar IRES cassettes containing 3-4 or 7-8 A's. As will be recognized, particularly in gene delivery, protein expression from a recombinant vector is important not only for in vitro protein production but also for therapeutic protein production in vivo. For example, Logg et al. (J. Virol. 75:6989-6998, 2001) describes an IRES that carries seven adenosine residues (As) instead of the 5-6 A's in the A bulge in the bifurcation region.

The optimized IRES cassette can be cloned into any number of art recognized vectors. Such vectors are described below, but include plasmids and viral vectors. For example, the disclosure contemplates an optimized IRES of the disclosure cloned into an expression vector wherein the optimized IRES is located just upstream (e.g., 0 to about 50 bp upstream) of a heterologous polynucleotide to be expressed. Of particular interest is the use of replication competent gamma retroviral vectors that are capable of infecting and spreading in mammalian tissue without the need for recombinant receptors or helper cells. Such RCR vectors include gamma retroviruses such as mo-MLV, MLV, GALV, FELV and the like. A typical gamma retrovirus comprises LTRs, gag, pol and env gene, and factors necessary for reverse transcription and integration into a host genome (e.g., psi factors). Modifications of the typical gamma retroviral vector have been performed for nearly 20 years including generating replication incompetent vectors, vectors carrying heterologous genes in various locations and vectors containing IRES cassettes. For example, Kasahara et al. describes the generation of a replication competent retroviral vector derived from MLV in U.S. Pat. No. 6,410,313 that carries an IRES cassette downstream of the env gene and upstream of the 3′ LTR. Gruber et al. (U.S. Pat. No. 8,722,867) describe a further optimized vector comprising an IRES cassette just downstream of the env gene and upstream of the 3′LTR. In Gruber et al. the IRES cassette shows an A-bulge of 7As in the JK bifurcation region.

The disclosure provides, in one embodiment, a replication competent gammaretroviral vector (RCR) comprising an optimized IRES cassette just downstream of the env gene and upstream of the 3′ LTR, wherein the optimized IRES of the optimized IRES cassette consists of an A-bulge in the bifurcation region of 5-6As. In a further embodiment, the RCR has increased stability, replication capacity and/or protein expression compared to a vector containing an A-bulge having 3-4 or 7-8A's.

The disclosure provides vectors having an A-bulge in the J-K bifurcation region consisting of 5-6A's compared to that found in prior replication competent retroviral vectors (e.g., see U.S. Patent Publ. Nos: 2011/0287020-A1; and 2011/0217267-A1, which show 7A's in the A-bulge, the disclosures of which are incorporated herein by reference). Unexpectedly the change in a single A (i.e., 7A's to 6A's) provides increased protein production compared to that of 7A's. Furthermore, the change of two A's (e.g., from 7 to 5 A's) results in increased vector stability. Thus, a vector comprising 5-6A's would have improved stability and/or protein expression of a heterologous gene linked to an IRES cassette having a “5A” or “6A” A-bulge compared to a vector having less than 5As or greater than 6As in the A-bulge.

The terms “vector”, “vector construct” and “expression vector” mean the vehicle by which a DNA or RNA sequence (e.g., a foreign gene) can be introduced into a host cell, so as to transform the host and promote expression (e.g., transcription and translation) of the introduced sequence. Vectors typically comprise the DNA or RNA of a transmissible agent, into which foreign DNA or RNA encoding a protein is inserted by restriction enzyme technology. A common type of vector is a “plasmid”, which generally is a self-contained molecule of double-stranded DNA that can readily accept additional (foreign) DNA and which can readily introduced into a suitable host cell. A large number of vectors, including plasmid and fungal vectors, have been described for replication and/or expression in a variety of eukaryotic and prokaryotic hosts. Non-limiting examples include pKK plasmids (Clonetech), pUC plasmids, pET plasmids (Novagen, Inc., Madison, Wis.), pRSET or pREP plasmids (Invitrogen, San Diego, Calif.), or pMAL plasmids (New England Biolabs, Beverly, Mass.), and many appropriate host cells, using methods disclosed or cited herein or otherwise known to those skilled in the relevant art. Recombinant cloning vectors will often include one or more replication systems for cloning or expression, one or more markers for selection in the host, e.g., antibiotic resistance, and one or more expression cassettes.

The terms “express” and “expression” mean allowing or causing the information in a gene or DNA sequence to become manifest, for example producing a protein by activating the cellular functions involved in transcription and translation of a corresponding gene, RNA or DNA sequence. A DNA or RNA sequence is expressed in or by a cell to form an “expression product” such as a protein. The expression product itself, e.g. the resulting protein, may also be said to be “expressed” by the cell. A polynucleotide or polypeptide is expressed recombinantly, for example, when it is expressed or produced in a foreign host cell under the control of a foreign or native promoter, or wherein a native gene in a native host cell is expressed under the control of a foreign promoter.

The disclosure provides modified retroviral vectors. The modified retroviral vectors can be derived from members of the retroviridae family. The Retroviridae family consists of three groups: the spumaviruses-(or foamy viruses) such as the human foamy virus (HFV); the lentiviruses, as well as visna virus of sheep; and the oncoviruses (although not all viruses within this group are oncogenic). The term “lentivirus” is used in its conventional sense to describe a genus of viruses containing reverse transcriptase. The lentiviruses include the “immunodeficiency viruses” which include human immunodeficiency virus (HIV) type 1 and type 2 (HIV-1 and HIV-2) and simian immunodeficiency virus (SIV). The oncoviruses have historically been further subdivided into groups A, B, C and D on the basis of particle morphology, as seen under the electron microscope during viral maturation. A-type particles represent the immature particles of the B- and D-type viruses seen in the cytoplasm of infected cells. These particles are not infectious. B-type particles bud as mature virion from the plasma membrane by the enveloping of intracytoplasmic A-type particles. At the membrane they possess a toroidal core of 75 nm, from which long glycoprotein spikes project. After budding, B-type particles contain an eccentrically located, electron-dense core. The prototype B-type virus is mouse mammary tumor virus (MMTV). No intracytoplasmic particles can be observed in cells infected by C-type viruses. Instead, mature particles bud directly from the cell surface via a crescent ‘C’-shaped condensation which then closes on itself and is enclosed by the plasma membrane. Envelope glycoprotein spikes may be visible, along with a uniformly electron-dense core. Budding may occur from the surface plasma membrane or directly into intracellular vacuoles. The C-type viruses are the most commonly studied and include many of the avian and murine leukemia viruses (MLV). Bovine leukemia virus (BLV), and the human T-cell leukemia virus types I and II (HTLV-I/II) are similarly classified as C-type particles because of the morphology of their budding from the cell surface. However, they also have a regular hexagonal morphology and more complex genome structures than the prototypic C-type viruses such as the murine leukemia viruses (MLV). D-type particles resemble B-type particles in that they show as ring-like structures in the infected cell cytoplasm, which bud from the cell surface, but the virion incorporate short surface glycoprotein spikes. The electron-dense cores are also eccentrically located within the particles. Mason Pfizer monkey virus (MPMV) is the prototype D-type virus.

Retroviruses have been classified in various ways but the nomenclature has been standardized in the last decade (see ICTVdB—The Universal Virus Database, v 4 on the World Wide Web (www) at ncbi.nlm.nih.gov/ICTVdb/ICTVdB/ and the text book “Retroviruses” Eds Coffin, Hughs and Varmus, Cold Spring Harbor Press 1997; the disclosures of which are incorporated herein by reference). In one embodiment, the replication competent retroviral vector can comprise an Orthoretrovirus or more typically a gamma retrovirus vector.

Retroviruses are defined by the way in which they replicate their genetic material. During replication the RNA is converted into DNA. Following infection of the cell a double-stranded molecule of DNA is generated from the two molecules of RNA which are carried in the viral particle by the molecular process known as reverse transcription. The DNA form becomes covalently integrated in the host cell genome as a provirus, from which viral RNAs are expressed with the aid of cellular and/or viral factors. The expressed viral RNAs are packaged into particles and released as infectious virion.

The retrovirus particle is composed of two identical RNA molecules. Each wild-type genome has a positive sense, single-stranded RNA molecule, which is capped at the 5′ end and polyadenylated at the 3′ tail. The diploid virus particle contains the two RNA strands complexed with gag proteins, viral enzymes (pol gene products) and host tRNA molecules within a ‘core’ structure of gag proteins. Surrounding and protecting this capsid is a lipid bilayer, derived from host cell membranes and containing viral envelope (env) proteins. The env proteins bind to a cellular receptor for the virus and the particle typically enters the host cell via receptor-mediated endocytosis and/or membrane fusion.

After the outer envelope is shed, the viral RNA is copied into DNA by reverse transcription. This is catalyzed by the reverse transcriptase enzyme encoded by the pol region and uses the host cell tRNA packaged into the virion as a primer for DNA synthesis. In this way the RNA genome is converted into the more complex DNA genome.

The double-stranded linear DNA produced by reverse transcription may, or may not, have to be circularized in the nucleus. The provirus now has two identical repeats at either end, known as the long terminal repeats (LTR). The termini of the two LTR sequences produces the site recognized by a pol product—the integrase protein—which catalyzes integration, such that the provirus is always joined to host DNA two base pairs (bp) from the ends of the LTRs. A duplication of cellular sequences is seen at the ends of both LTRs, reminiscent of the integration pattern of transposable genetic elements. Retroviruses can integrate their DNAs at many sites in host DNA, but different retroviruses have different integration site preferences. HIV-1 and simian immunodeficiency virus DNAs preferentially integrate into expressed genes, murine leukemia virus (MLV) DNA preferentially integrates near transcriptional start sites (TSSs), and avian sarcoma leukosis virus (ASLV) and human T cell leukemia virus (HTLV) DNAs integrate nearly randomly, showing a slight preference for genes (Derse D, et al. (2007) Human T-cell leukemia virus type 1 integration target sites in the human genome: comparison with those of other retroviruses. J Virol 81:6731-6741; Lewinski M K, et al. (2006) Retroviral DNA integration: viral and cellular determinants of target-site selection. PLoS Pathog 2:e601).

Transcription, RNA splicing and translation of the integrated viral DNA is mediated by host cell proteins. Variously spliced transcripts are generated. In the case of the human retroviruses HIV-1/2 and HTLV-I/II viral proteins are also used to regulate gene expression. The interplay between cellular and viral factors is a factor in the control of virus latency and the temporal sequence in which viral genes are expressed.

Retroviruses can be transmitted horizontally and vertically. Efficient infectious transmission of retroviruses requires the expression on the target cell of receptors which specifically recognize the viral envelope proteins, although viruses may use receptor-independent, nonspecific routes of entry at low efficiency. Normally a viral infection leads to a single or few copies of viral genome per cell because of receptor masking or down-regulation that in turn leads to resistance to superinfection (Ch3 p104 in “Retroviruses”, J M Coffin, S H Hughes, & H E Varmus 1997 Cold Spring Harbor Laboratory Press, Cold Spring Harbor N.Y.; Fan et al. J. Virol 28:802, 1978). In addition, the target cell type must be able to support all stages of the replication cycle after virus has bound and penetrated. Vertical transmission occurs when the viral genome becomes integrated in the germ line of the host. The provirus will then be passed from generation to generation as though it were a cellular gene. Hence endogenous proviruses become established which frequently lie latent, but which can become activated when the host is exposed to appropriate agents.

In many situations for using a recombinant replication competent retrovirus therapeutically, it is advantageous to have high levels of expression of the transgene that is encoded by the recombinant replication competent retrovirus. For example, with a prodrug activating gene such as the cytosine deaminase gene it is advantageous to have higher levels of expression of the CD protein in a cell so that the conversion of the prodrug 5-FC to 5-FU is more efficient. Similarly high levels of expression of siRNA or shRNA lead to more efficient suppression of target gene expression. Also for cytokines or single chain antibodies (scAbs) it is usually advantageous to express high levels of the cytokine or scAb. In addition, in the case that there are mutations in some copies of the vector that inactivate or impair the activity of the vector or transgene, it is advantageous to have multiple copies of the vector in the target cell as this provides a high probability of efficient expression of the intact transgene. The disclosure provides recombinant replication competent retroviruses capable of infecting a target cell or target cell population multiple times resulting in an average number of copies/diploid genome of 5 or greater. The disclosure also provides methods of testing for this property. Also provided are methods of treating a cell proliferative disorder, using a recombinant replication competent retrovirus capable of infecting a target cell or target cell population multiple times resulting in an average number of copies/diploid genome of 5 or greater.

As mentioned above, the integrated DNA intermediate is referred to as a provirus. Prior gene therapy or gene delivery systems use methods and retroviruses that require transcription of the provirus and assembly into infectious virus while in the presence of an appropriate helper virus or in a cell line containing appropriate sequences enabling encapsidation without coincident production of a contaminating helper virus. As described below, a helper virus is not required for the production of the recombinant retrovirus of the disclosure, since the sequences for encapsidation are provided in the genome thus providing a replication competent retroviral vector for gene delivery or therapy.

Other existing replication competent retroviral vectors also tend to be unstable and lose sequences during horizontal or vertical transmission to an infected cell or host cell and during replication. This may be due in-part from the presence of extra nucleotide sequences that include repeats or which reduce the efficiency of a polymerase.

The retroviral genome and the proviral DNA of the disclosure have at least three genes: the gag, the pol, and the env, these genes may be flanked by one or two long terminal (LTR) repeat, or in the provirus are flanked by two long terminal repeat (LTR) and sequences containing cis-acting sequences such as psi. The gag gene encodes the internal structural (matrix, capsid, and nucleocapsid) proteins; the pol gene encodes the RNA-directed DNA polymerase (reverse transcriptase), protease and integrase; and the env gene encodes viral envelope glycoproteins. The 5′ and/or 3′ LTRs serve to promote transcription and polyadenylation of the virion RNAs. The LTR contains all other cis-acting sequences necessary for viral replication. Lentiviruses have additional genes including vif, vpr, tat, rev, vpu, nef, and vpx (in HIV-1, HIV-2 and/or SIV).

Adjacent to the 5′ LTR are sequences necessary for reverse transcription of the genome (the tRNA primer binding site) and for efficient encapsidation of viral RNA into particles (the Psi site). If the sequences necessary for encapsidation (or packaging of retroviral RNA into infectious virion) are missing from the viral genome, the result is a cis defect which prevents encapsidation of genomic viral RNA. This type of modified vector is what has typically been used in prior gene delivery systems (i.e., systems lacking elements which are required for encapsidation of the virion) as ‘helper’ elements providing viral proteins in trans that package a non-replicating, but packageable, RNA genome.

The disclosure provides vectors that contain an optimized IRES. The optimized IRES is typically linked to a heterologous polynucleotide encoding, for example, a cytosine deaminase or mutant thereof, a thymidine kinase or mutant thereof, an miRNA or siRNA, a cytokine, an antibody binding domain etc., that can be delivered to a cell or subject. In one embodiment, the vector is a viral vector. The viral vector can be an adenoviral vector, a measles vector, a herpes vector, a retroviral vector (including a lentiviral vector), a rhabdoviral vector such as a Vesicular Stomatitis viral vector, a reovirus vector, a Seneca Valley Virus vector, a poxvirus vector (including animal pox or vaccinia derived vectors), a parvovirus vector (including an AAV vector), an alphavirus vector or other viral vector known to one skilled in the art (see also, e.g., Concepts in Genetic Medicine, ed. Boro Dropulic and Barrie Carter, Wiley, 2008, Hoboken, N.J. ; The Development of Human Gene Therapy, ed. Theodore Friedmann, Cold Springs Harbor Laboratory Press, Cold springs Harbor, N.Y., 1999; Gene and Cell Therapy, ed. Nancy Smyth Templeton, Marcel Dekker Inc., New York, N.Y., 2000 and Gene Therapy: Therapeutic Mechanism and Strategies, ed. Nancy Smyth Templetone and Danilo D Lasic, Marcel Dekker, Inc., New York, N.Y., 2000; the disclosures of which are incorporated herein by reference).

In one embodiment, the retroviral genome of the disclosure contains an optimized IRES comprising a cloning site downstream of the optimized IRES for insertion of a desired/heterologous polynucleotide. In one embodiment, the optimized IRES is located 3′ to the env gene in a retroviral vector, but 5′ to the desired heterologous polynucleotide and 5′ to the 3′ LTR. In all of the foregoing embodiments, the optimized IRES comprises an A-bulge with 5-6A's. A heterologous polynucleotide encoding a desired polypeptide may be operably linked to the optimized IRES.

In one embodiment, the viral vector can be a replication competent retroviral vector obtained or derived from a gammaretrovirus capable of infecting replicating mammalian cells. The replication competent retroviral vector comprises an optimized internal ribosomal entry site (IRES) comprising an A-bulge consisting of 5-6 A's located 5′ to a heterologous polynucleotide encoding, e.g., a cytosine deaminase (SEQ ID NO:3), thymidine kinase (SEQ ID NO:37), miRNA, siRNA, cytokine, receptor, antibody or the like. When the heterologous polynucleotide encodes a non-translated RNA such as siRNA, miRNA or RNAi then an IRES is not necessary, but may be included for another translated polynucleotide. In one embodiment, an optimized IRES cassette containing the heterologous polynucleotide is 3′ to a ENV polynucleotide of a retroviral vector, but 5′ to the 3′ LTR. In one embodiment the viral vector is a retroviral vector capable of infecting target cells multiple times (e.g., 5 or more per diploid cell).

The disclosure provides replication competent retroviral vectors having increased stability relative to prior retroviral vectors and containing an optimized IRES having 5-6A's in the A-bulge. Such increased stability during infection and replication is important for the treatment of cell proliferative disorders. In addition, the increased protein expression from the optimized A-bulge provides additional delivery of therapeutic proteins to a target cell/tissue. The combination of transduction efficiency, transgene stability, transgene expression and target selectivity is provided by the replication competent retrovirus. The compositions and methods provide insert stability and maintain transcription activity of the transgene and the translational viability of the encoded polypeptide.

Depending upon the intended use of a vector or the retroviral vector of the disclosure any number of heterologous polynucleotide or nucleic acid sequences may be inserted into the vector or retroviral vector. For example, for in vitro studies commonly used marker genes or reporter genes may be used, including, antibiotic resistance and fluorescent molecules (e.g., GFP). Additional polynucleotide sequences encoding any desired polypeptide sequence may also be inserted into the vector of the disclosure. Where in vivo delivery of a heterologous nucleic acid sequence is sought both therapeutic and non-therapeutic sequences may be used. For example, the heterologous sequence can encode a therapeutic molecule including antisense molecules (miRNA, siRNA) or ribozymes directed to a particular gene associated with a cell proliferative disorder or other gene-associated disease or disorder, the heterologous sequence can be a suicide gene (e.g., HSV-tk or PNP or cytosine deaminase; either modified or unmodified, humanized or non-humanized), a growth factor or a therapeutic protein (e.g., Factor IX, IL2, and the like). Other therapeutic proteins applicable to the disclosure are easily identified in the art.

In one embodiment, the heterologous polynucleotide within the vector comprises a cytosine deaminase that has been optimized for expression in a human cell. In a further embodiment, the cytosine deaminase comprises a sequence that has been human codon optimized and comprises mutations that increase the cytosine deaminase's stability (e.g., reduced degradation or increased thermo-stability) compared to a wild-type cytosine deaminase (see, e.g., SEQ ID NO:4). In yet another embodiment, the heterologous polynucleotide encodes a fusion construct comprising a cytosine deaminase (either human codon optimized or non-optimized, either mutated or non-mutated) operably linked to a polynucleotide encoding a polypeptide having UPRT or OPRT activity (see, e.g., SEQ ID NO:11, 13, 15 and 17). Examples of such polypeptides having cytosine deaminase and polynucleotides encoding such polypeptides can be found in International Publication No. WO 2010/045002, which is incorporated herein by reference.

In another embodiment, a vector or replication competent retroviral vector can comprise a heterologous polynucleotide encoding a polypeptide comprising a cytosine deaminase (as described herein) and may further comprise a polynucleotide comprising a miRNA or siRNA molecule either as part of the primary transcript from the viral promoter or linked to a promoter, which can be cell-type or tissue specific.

In yet further embodiments, the heterologous polynucleotide may comprise a cytokine such as an interleukin, interferon gamma or the like. Cytokines that may expressed from a retroviral vector of the disclosure include, but are not limited to, IL-1alpha, IL-1beta, IL-2 (SEQ ID NO:40), IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, and IL-21, anti-CD40, CD40L, IFN-gamma (human—SEQ ID NO:38; mouse—SEQ ID NO:39) and TNF-alpha, soluble forms of TNF-alpha, lymphotoxin-alpha (LT-alpha, also known as TNF-beta), LT-beta (found in complex heterotrimer LT-alpha2-beta), OPGL, FasL, CD27L, CD30L, CD40L, 4-1BBL, DcR3, OX40L, TNF-gamma (International Publication No. WO 96/14328), AIM-I (International Publication No. WO 97/33899), endokine-alpha (International Publication No. WO 98/07880), OPG, and neutrokine-alpha (International Publication No. WO 98/18921, OX40, and nerve growth factor (NGF), and soluble forms of Fas, CD30, CD27, CD40 and 4-IBB, TR2 (International Publication No. WO 96/34095), DR3 (International Publication No. WO 97/33904), DR4 (International Publication No. WO 98/32856), TR5 (International Publication No. WO 98/30693), TRANK, TR9 (International Publication No. WO 98/56892), TR10 (International Publication No. WO 98/54202), 312C2 (International Publication No. WO 98/06842), and TR12, and soluble forms CD154, CD70, and CD153. Angiogenic proteins may be useful in some embodiments, particularly for protein production from cell lines. Such angiogenic factors include, but are not limited to, Glioma Derived Growth Factor (GDGF), Platelet Derived Growth Factor-A (PDGF-A), Platelet Derived Growth Factor-B (PDGF-B), Placental Growth Factor (PIGF), Placental Growth Factor-2 (PIGF-2), Vascular Endothelial Growth Factor (VEGF), Vascular Endothelial Growth Factor-A (VEGF-A), Vascular Endothelial Growth Factor-2 (VEGF-2), Vascular Endothelial Growth Factor B (VEGF-3), Vascular Endothelial Growth Factor B-1 86 (VEGF-B186), Vascular Endothelial Growth Factor-D (VEGF-D), Vascular Endothelial Growth Factor-D (VEGF-D), and Vascular Endothelial Growth Factor-E (VEGF-E). Fibroblast Growth Factors may be delivered by a vector of the disclosure and include, but are not limited to, FGF-1, FGF-2, FGF-3, FGF-4, FGF-5, FGF-6, FGF-7, FGF-8, FGF-9, FGF-10, FGF-11, FGF-12, FGF-13, FGF-14, and FGF-15. Hematopoietic growth factors may be delivered using vectors of the disclosure, such growth factors include, but are not limited to, granulocyte macrophage colony stimulating factor (GM-CSF) (sargramostim), granulocyte colony stimulating factor (G-CSF) (filgrastim), macrophage colony stimulating factor (M-CSF, CSF-1) erythropoietin (epoetin alfa), stem cell factor (SCF, c-kit ligand, steel factor), megakaryocyte colony stimulating factor, PIXY321 (a GMCSF/IL-3) fusion protein and the like.

MicroRNAs (miRNA) are small, non-coding RNAs. They are located within introns of coding or non-coding gene, exons of non-coding genes or in inter-genic regions. miRNA genes are transcribed by RNA polymerase II that generate precursor polynucleotides called primary precursor miRNA (pri-miRNA). The pri-miRNA in the nucleus is processed by the ribonuclease Drosha to produce the miRNA precursor (pre-miRNA) that forms a short hairpin structure. Subsequently, pre-miRNA is transported to the cytoplasm via Exportin 5 and further processed by another ribonuclease called Dicer to generate an active, mature miRNA.

A mature miRNA is approximately 21 nucleotides in length. It exerts in function by binding to the 3′ untranslated region of mRNA of targeted genes and suppressing protein expression either by repression of protein translation or degradation of mRNA. miRNA are involved in biological processes including development, cell proliferation, differentiation and cancer progression. Studies of miRNA profiling indicate that some miRNA expressions are tissue specific or enriched in certain tissues. For example, miR-142-3p, miR-181 and miR-223 expressions have demonstrated to be enriched in hematopoietic tissues in human and mouse (Baskerville et al., 2005 RNA 11, 241-247; Chen et al., 2004 Science 303, 83-86). The target sequence of miR-142-3p is shown in SEQ ID NO:35. The target of miR-142-3p4X is shown in SEQ ID NO:36.

Some miRNAs have been observed to be up-regulated (oncogenic miRNA) or down-regulated (repressor)in several tumors (Spizzo et al., 2009 Cell 137, 586e1). For example, miR-21 is overexpressed in glioblastoma, breast, lung, prostate, colon, stomach, esophageal, and cervical cancer, uterine leiomyosarcoma, DLBCL, head and neck cancer. In contrast, members of let-7 have reported to be down-regulated in glioblastoma, lung, breast, gastric, ovary, prostate and colon cancers. Re-establishment of homeostasis of miRNA expression in cancer is an imperative mechanism to inhibit or reverse cancer progression.

As a consequence of the vital functions modulated by miRNAs in cancers, focus in developing potential therapeutic approaches has been directed toward antisense-mediated inhibition (antigomers) of oncogenic miRNAs. However, miRNA replacement might represent an equally efficacious strategy. In this approach, the most therapeutically useful miRNAs are the ones expressed at low levels in tumors but at high level, and therefore tolerated, in normal tissues.

miRNAs that are down-regulated in cancers could be useful as anticancer agents. Examples include mir-128-1/2 (SEQ ID NO:31 and 32 respectively), let-7, miR-26, miR-124, and miR-137 (Esquela-Kerscher et al., 2008 Cell Cycle 7, 759-764; Kumar et al., 2008 Proc Natl Acad Sci USA 105, 3903-3908; Kota et al., 2009 Cell 137, 1005-1017; Silber et al., 2008 BMC Medicine 6:14 1-17). miR-128 expression has reported to be enriched in the central nervous system and has been observed to be down-regulated in glioblastomas (Sempere et al., 2004 Genome Biology 5:R13.5-11; Godlewski et al., 2008 Cancer Res 68: (22) 9125-9130). miR-128 is encoded by two distinct genes, miR-128-1 and miR-128-2. Both are processed into identical mature sequence. Bmi-1 and E2F3a have been reported to be the direct targets of miR-128 (Godlewski et al., 2008 Cancer Res 68:(22) 9125-9130; Zhang et al., 2009 J. Mol Med 87:43-51). In addition, Bmi-1 expression has been observed to be up-regulated in a variety of human cancers, including gliomas, mantle cell lymphomas, non-small cell lung cancer B-cell non-Hodgkin's lymphoma, breast, colorectal and prostate cancer. Furthermore, Bmi-1 has been demonstrated to be required for the self-renewal of stem cells from diverse tissues, including neuronal stem cells as well as “stem-like” cell population in gliomas.

Although there have been a number of in vitro demonstrations of the possibilities of miRNA mediated inhibition of cellular function, it has been difficult to deliver these as oligonucleotides or in viral vectors as efficiently as necessary to have in vivo effects (e.g., Li et al., Cell Cycle 5:2103-2109 2006), as has been true for other molecules.

Replication-defective retroviral and lentiviral vectors have been used to stably express pri-miRNA by a polymerase II promoter such as CMV or LTR and demonstrated production of mature miRNA. The, incorporation of type III RNA polymerase III promoters such as the U6 and the H1 promoter in non-replicative retroviral and lentiviral vectors has been used widely to express functional small interference RNA (siRNA) producing a short hairpin structured RNA (Bromberg-White et: al., 2004 J Virol 78:9, 4914-4916; Sliva et al., 2006 Virology 351, 218-225; Haga et al., 2006, Transplant Proc 38(10):3184-8). The loop sequence is cleaved by Dicer producing the mature siRNAs that are 21-22 nucleotides in length. shRNA can be stably expressed in cells to down-regulate target gene expression. SEQ ID NO:33 and 34 comprise a pre-miR-128 linked to an H1 promoter.

In another embodiment, an optimized IRES comprising 5-6A's in the A-bulge can be used in combination with a core promoter, wherein an optimized IRES is operably linked to a first heterologous coding sequence and the core promoter or minipromoter is linked to a second heterologous coding sequence or an siRNA, miRNA, or shRNA sequence (see, e.g., WO 2014/066700, incorporated herein by reference).

As used herein, a “core promoter” refers to a minimal promoter comprising about 50-100 bp and lacks enhancer elements. Such core promoters include, but are not limited to, SCP1, AdML and CMV core promoters. More particularly, where a core-promoter cassette is present a second cassette (e.g., a second mini-promoter cassette, a polIII promoter cassette or IRES cassette) will be present. In some embodiments, a vector comprising a cassette with a core promoter specifically excludes the use of SCP1, AdML and CMV core promoters, but rather utilize designer core promoters as described further herein and below.

Core promoters include certain viral promoters. Viral promoters, as used herein, are promoters that have a core sequence but also usually some further accessory elements. For example, the early promoter for SV40 contains three types of elements: a TATA box, an initiation site and a GC repeat (Barrera-Saldana et al., EMBO J, 4:3839-3849, 1985; Yaniv, Virology, 384:369-374, 2009). The TATA box is located approximately 20 base-pairs upstream from the transcriptional start site. The GC repeat regions is a 21 base-pair repeat containing six GC boxes and is the site that determines the direction of transcription. This core promoter sequence is around 100 bp. Adding an additional 72 base-pair repeats, thus making it a “mini-promoter,” is useful as a transcriptional enhancer that increase the functionality of the promoter by a factor of about 10. When the SP1 protein interacts with the 21 bp repeats it binds either the first or the last three GC boxes. Binding of the first three initiates early expression, and binding of the last three initiates late expression. The function of the 72 bp repeats is to enhance the amount of stable RNA and increase the rate of synthesis. This is done by binding (dimerization) with the AP1 (activator protein 1) to give a primary transcript that is 3′ polyadenylated and 5′ capped. Other viral promoters, such as the Rous Sarcoma Virus (RSV), the HBV X gene promoter, and the Herpes Thymidine kinase core promoter can also be used as the basis for selection desired function.

A core promoter typically encompasses −40 to +40 relative to the +1 transcription start site (Juven-Gershon and Kadonaga, Dev. Biol. 339:225-229, 2010), which defines the location at which the RNA polymerase II machinery initiates transcription. Typically, RNA polymerase II interacts with a number of transcription factors that bind to DNA motifs in the promoter. These factors are commonly known as “general” or “basal” transcriptions factors and include, but are not limited to, TFIIA (transcription factor for RNA polymerase IIA), TFIIB, TFIID, TFIIE, TFIIF, and TFIIH. These factors act in a “general” manner with all core promoters; hence they are often referred to as the “basal” transcription factors.

Juven-Gershon et al., (Nat. Methods, 3(11):917-922, 2006), describe elements of core promoters. For example, the pRC/CMV core promoter consists of a TATA box and is 81 bp in length; the CMV core promoter consists of a TATA box and a initiator site; while the SCP synthetic core promoters (SCP1 and SCP2) consist of a TATA box, an Inr (initiator), an MTE site (Motif Ten Element), and a DPE site (Downstream promoter element) and is about 81 bp in length. The SCP synthetic promoter has improved expression compared to the simple pRC/CMV core promoter.

As used herein a “mini-promoter” or “small promoter” refers to a regulatory domain that promotes transcription of an operably linked gene or coding nucleic acid sequence. The mini-promoter, as the name implies, includes the minimal amount of elements necessary for effective transcription and/or translation of an operably linked coding sequence. A mini-promoter can comprise a “core promoter” in combination with additional regulatory elements or a “modified core promoter”. Typically, the mini-promoter or modified core promoter will be about 100-600 bp in length while a core promoter is typically less than about 100 bp (e.g., about 70-80 bp). In other embodiments, where a core promoter is present, the cassette will typically comprise an enhancer element or another element either upstream or downstream of the core promoter sequence that facilitates expression of an operably linked coding sequence above the expression levels of the core promoter alone.

Accordingly, the disclosure provides mini-promoters (e.g., modified core promoters) derived from cellular elements as determined for “core promoter” elements (<100, <200, <400 or <600 bp) that allow ubiquitous expression at significant levels in target cells and are useful for stable incorporation into vectors, in general, and replicating retroviral vectors, in particular, to allow efficient expression of transgenes. Also provided are mini-promoters comprising core promoters plus minimal enhancer sequences and/or Kozak sequences to allow better gene expression compared to a core-promoter lacking such sequences that are still under 200, 400 or 600 bp. Such mini-promoters include modified core promoters and naturally occurring tissue specific promoters such as the elastin promoter (specific for pancreatic acinar cells, (204 bp; Hammer et al., Mol Cell Biol., 7:2956-2967, 1987) and the promoter from the cell cycle dependent ASK gene from mouse and man (63-380 bp; Yamada et al., J. Biol. Chem., 277: 27668-27681, 2002). Ubiquitously expressed small promoters also include viral promoters such as the SV40 early and late promoters (about 340 bp), the RSV LTR promoter (about 270 bp) and the HBV X gene promoter (about 180 bp) (e.g., R Anish et al., PLoS One, 4: 5103, 2009) that has no canonical “TATTAA box” and has a 13 bp core sequence of 5′-CCCCGTTGCCCGG-3′ (SEQ ID NO:43). In yet other embodiments, the therapeutic cassette comprising at least one mini-promoter cassette will have expression levels that exceed, are about equal to, or about about 1 fold to 2.5 fold less than the expression levels of an IRES cassette present in an RRV.

Transcription from a core- or mini-promoter occurs through the interaction of various elements. In focused transcription, for example, there is either a single major transcription start site or several start sites within a narrow region of several nucleotides. Focused transcription is the predominant mode of transcription in simpler organisms. In dispersed transcription, there are several weak transcription start sites over a broad region of about 50 to 100 nucleotides. Dispersed transcription is the most common mode of transcription in vertebrates. For instance, dispersed transcription is observed in about two-thirds of human genes. In vertebrates, focused transcription tends to be associated with regulated promoters, whereas dispersed transcription is typically observed in constitutive promoters in CpG islands.

TABLE 1
Binding sites that can contribute to
a focused core promoter (almost
always with a “TATA box and a single
transcription start site (TSS)),
or a dispersed promoter without a
TATA box, usually with a DPE element
(see R. Dickstein, Trasncription, 2(5):201-206,
2011; Juven-Gershon et al., Nat. Methods,
2006, supra). Symbols for nucleotides follow the
international convention (world wide web:
chem.qmul.ac.uk/iubmb/misc/naseq.html).

| Tran- | | Binding site wrt to |
|---|---|---|
| scription | | transcription start |
| factor | Full name | site (TSS +1) |
| BREu | TFIIB | Upstream of TATA Box, |
| | recognition | SSRCGCC |
| | element, | |
| | upstream | |
| TATA box | TATA box | T at -31/-30 TATAWAAR, |
| | | key focused promoter |
| | | element |
| BREd | TFIIB | -23 to -17 RTDKKKK |
| | recognition | |
| | element, | |
| | downstream | |
| XCPE1 | HBV X core | -8 to +2 DSGYGGRASM |
| | promoter | from HBV Xgene |
| | element 1 | |
| XCPE2 | HBV X core | VCYCRTTRCMY from HBV |
| | promoter | Xgene |
| | element 2 | |
| Inr | initiator | -2 to +4 YYANWYY |
| DCE SI | Downstream core | +6 to +11 CTTC |
| | element site 1 | |
| DCE SII | Downstream core | +16 to +21 CTGT |
| | element site II | |
| DCE SIII | Downstream core | +30 to +34 AGC |
| | element site III | |
| MTE | Motif ten | +18 to +27 CSARCSSAAC |
| | element | mostly in Drosophila |
| DPE | Downstream | +28 to +33 RGWYVT common |
| | promoter | in Drosophila, key |
| | element | dispersed promoter |
| | | element |

Table 2 sets forth oligonucleotides that can be used to construct and clone enhancer elements into core promoter regions. As mentioned above, the modified/optimized core promoters of the disclosure can include a core sequence with the addition of elements from Table 1 and may further include enhancers cloned as set forth in Table 2. In doing so, the size of the mini-promoter may be increased. However, the final mini-promoter should not exceed 600 bp and will typically be about 100 bp, 200 bp, 300 bp, 400 bp, 500 bp and any integer there between.

TABLE 2
Oligonucleotides Used for
Constructing Enhancer segments.

| | Oligo- | Motif | |
|---|---|---|---|
| No. | nucleotide | Sequence | Reference |
| 1 | AP-1 | 5′-TGTCTCA | Hallahanet al. Int. |
| | | G-3′ | J. Radiat. |
| | | | Oncol. Biol. Phys. |
| | | | 36:355-360 1996. |
| 2 | CArG | 5′-CCATATA | Datta et al. Proc. |
| | | AGG-3′ | Natl. Acad. Sci. |
| | | (SEQ ID | USA 89:10149-10153. |
| | | NO: 44) | 1992 |
| 3 | NF-κB1 | 5′-GGAAATC | Ueda et al. FEBS |
| | | CCC-3′ | Lett. 491:40-44 |
| | | (SEQ ID | 2001 |
| | | NO: 45) | |
| 4 | NF-κB2 | 5′-GGAAAGT | Kanno et al. EMBO |
| | | CCCC-3′ | J. 8:4205-4214 |
| | | (SEQ ID | 1989 |
| | | NO: 46) | |
| 5 | NF-κB3 | 5′-GGAGTTC | Hong et al. J. |
| | | CC-3′ | Biol. Chem. 275: |
| | | | 18022-18028 2000. |
| 6 | NF-Y | 5′-CATTGG | Hu et al. J. Biol. |
| | | G-3′ | Chem. 275:2979- |
| | | | 2985 2000. |

AP-1, activating protein-1;
NF-κB, nuclear factor κB.

In one embodiment, the disclosure provides a recombinant replication competent retrovirus capable of infecting a non-dividing host cell, a host dividing cell, or a host cell having a cell proliferative disorder. The recombinant replication competent retrovirus of the disclosure comprises a polynucleotide sequence encoding a viral GAG, a viral POL, a viral ENV, a heterologous polynucleotide preceded by an optimized internal ribosome entry site (IRES) having 5-6 A's in the A-bulge of the IRES encapsulated within a virion.

Generally, the recombinant vector of the disclosure is capable of transferring a nucleic acid sequence into a target cell. The phrase “non-dividing” cell refers to a cell that does not go through mitosis. Non-dividing cells may be blocked at any point in the cell cycle, (e.g., G₀/G₁, G_{1/5}, G_{2/M}), so long as the cell is not actively dividing. For ex vivo infection, a dividing cell can be treated to block cell division by standard techniques used by those of skill in the art, including, irradiation, aphidocolin treatment, serum starvation, and contact inhibition. However, it should be understood that ex vivo infection is often performed without blocking the cells since many cells are already arrested (e.g., stem cells). For example, a recombinant lentivirus vector is capable of infecting non-dividing cells. Examples of pre-existing non-dividing cells in the body include neuronal, muscle, liver, skin, heart, lung, and bone marrow cells, and their derivatives. For dividing cells onco-retroviral vectors can be used.

By “dividing” cell is meant a cell that undergoes active mitosis, or meiosis. Such dividing cells include stem cells, skin cells (e.g., fibroblasts and keratinocytes), gametes, and other dividing cells known in the art. Of particular interest and encompassed by the term dividing cell are cells having cell proliferative disorders, such as neoplastic cells. The term “cell proliferative disorder” refers to a condition characterized by an abnormal number of cells. The condition can include both hypertrophic (the continual multiplication of cells resulting in an overgrowth of a cell population within a tissue) and hypotrophic (a lack or deficiency of cells within a tissue) cell growth or an excessive influx or migration of cells into an area of a body. The cell populations are not necessarily transformed, tumorigenic or malignant cells, but can include normal cells as well. Cell proliferative disorders include disorders associated with an overgrowth of connective tissues, such as various fibrotic conditions, including scleroderma, arthritis and liver cirrhosis. Cell proliferative disorders include neoplastic disorders such as head and neck carcinomas. Head and neck carcinomas would include, for example, carcinoma of the mouth, esophagus, throat, larynx, thyroid gland, tongue, lips, salivary glands, nose, paranasal sinuses, nasopharynx, superior nasal vault and sinus tumors, esthesioneuroblastoma, squamous cell cancer, malignant melanoma, sinonasal undifferentiated carcinoma (SNUC), brain (including glioblastomas) or blood neoplasia. Also included are carcinoma's of the regional lymph nodes including cervical lymph nodes, prelaryngeal lymph nodes, pulmonary juxtaesophageal lymph nodes and submandibular lymph nodes (Harrison's Principles of Internal Medicine (eds., Isselbacher, et al., McGraw-Hill, Inc., 13th Edition, pp1850-1853, 1994). Other cancer types, include, but are not limited to, lung cancer, colon-rectum cancer, breast cancer, prostate cancer, urinary tract cancer, uterine cancer lymphoma, oral cancer, pancreatic cancer, leukemia, melanoma, stomach cancer, skin cancer and ovarian cancer. The cell proliferative disease also includes rheumatoid arthritis (O'Dell NEJM 350:2591 2004)and other auto-immune disorders (Mackay et al NEJM 345:340 2001) that are often characterized by inappropriate proliferation of cells of the immune system.

In other embodiments, host cells transfected with a replication competent retroviral vector of the disclosure are provided. Host cells include eukaryotic cells such as yeast cells, insect cells, or animal cells. Host cells also include prokaryotic cells such as bacterial cells. In other embodiments, the host cells have been modified or selected to be continuously grown in serum free suspension (see, e.g., U.S. Patent Publ. No. 2012/0087894-A1, which is incorporated herein by reference).

Also provided are engineered host cells that are transduced (transformed or transfected) with a vector provided herein (e.g., a replication competent retroviral vector). The engineered host cells can be cultured in conventional nutrient media modified as appropriate for activating promoters, selecting transformants, or amplifying a coding polynucleotide. Culture conditions, such as temperature, pH and the like, are those previously used with the host cell selected for expression, and will be apparent to those skilled in the art and in the references cited herein, including, e.g., Sambrook, Ausubel and Berger, as well as e.g., Freshney (1994) Culture of Animal Cells: A Manual of Basic Technique, 3rd ed. (Wiley-Liss, New York) and the references cited therein.

Examples of appropriate expression hosts include: mammalian cells such as CHO, COS, BHK, HEK 293 br Bowes melanoma etc. Typically human cells or cell lines will be used; however, it may be desirable to clone vectors and polynucleotides of the disclosure into non-human host cells for purposes of sequencing, amplification and cloning.

In another embodiment, a targeting polynucleotide sequence is included as part of a recombinant retroviral vector of the disclosure. The targeting polynucleotide sequence is a targeting ligand (e.g., peptide hormones such as heregulin, a single-chain antibody, a receptor or a ligand for a receptor), a tissue-specific or cell-type specific regulatory element (e.g., a tissue-specific or cell-type specific promoter or enhancer), or a combination of a targeting ligand and a tissue-specific/cell-type specific regulatory element. The targeting ligand is operably linked to the env protein of the retrovirus, creating a chimeric retroviral env protein. The viral GAG, viral POL and viral ENV proteins can be derived from any suitable retrovirus (e.g., MLV or lentivirus-derived). In another embodiment, the viral ENV protein is non-retrovirus-derived (e.g., CMV or VSV).

In one embodiment, the retroviral vector is targeted to the cell by binding to cells having a molecule on the external surface of the cell. This method of targeting the retrovirus utilizes expression of a targeting ligand on the coat of the retrovirus to assist in targeting the virus to cells or tissues that have a receptor or binding molecule which interacts with the targeting ligand on the surface of the retrovirus. After infection of a cell by the virus, the virus injects its nucleic acid into the cell and the retrovirus genetic material can integrate into the host cell genome.

Thus, the disclosure includes in one embodiment, a chimeric env protein comprising a retroviral ENV protein operably linked to a targeting polypeptide. The targeting polypeptide can be a cell specific receptor molecule, a ligand for a cell specific receptor, an antibody or antibody fragment to a cell specific antigenic epitope or any other ligand easily identified in the art which is capable of binding or interacting with a target cell. Examples of targeting polypeptides or molecules include bivalent antibodies using biotin-streptavidin as linkers (Etienne-Julan et al., J. Of General Virol., 73, 3251-3255 (1992); Roux et al., Proc. Natl. Acad. Sci USA 86, 9079-9083 (1989)), recombinant virus containing in its envelope a sequence encoding a single-chain antibody variable region against a hapten (Russell et al., Nucleic Acids Research, 21, 1081-1085 (1993)), cloning of peptide hormone ligands into the retrovirus envelope (Kasahara et al., Science, 266, 1373-1376 (1994)), chimeric EPO/env constructs (Kasahara et al., 1994), single-chain antibody against the low density lipoprotein (LDL) receptor in the ecotropic MLV envelope, resulting in specific infection of HeLa cells expressing LDL receptor (Somia et al., Proc. Natl. Acad. Sci USA, 92, 7570-7574 (1995)), similarly the host range of ALV can be altered by incorporation of an integrin ligand, enabling the virus to now cross species to specifically infect rat glioblastoma cells (Valsesia-Wittmann et al., J. Virol. 68, 4609-4619 (1994)), and Dornberg and co-workers (Chu and Dornburg, J. Virol 69, 2659-2663 (1995); M. Engelstadter et al.Gene Therapy 8,1202-1206 (2001)) have reported tissue-specific targeting of spleen necrosis virus (SNV), an avian retrovirus, using envelopes containing single-chain antibodies directed against tumor markers.

In one embodiment, the recombinant retrovirus of the disclosure is genetically modified in such a way that the virus is targeted to a particular cell type (e.g., smooth muscle cells, hepatic cells, renal cells, fibroblasts, keratinocytes, mesenchymal stem cells, bone marrow cells, chondrocyte, epithelial cells, intestinal cells, mammary cells, neoplastic cells, glioma cells, neuronal cells and others known in the art) such that the recombinant genome of the retroviral vector is delivered to a target non-dividing, a target dividing cell, or a target cell having a cell proliferative disorder.

In another embodiment, targeting uses cell- or tissue-specific regulatory elements to promote expression and transcription of the viral genome in a targeted cell which actively utilizes the regulatory elements, as described more fully below. The transferred retrovirus genetic material is then transcribed and translated into proteins within the host cell. The targeting regulatory element is typically linked to the 5′ and/or 3′ LTR, creating a chimeric LTR.

The disclosure provides in one embodiment a replication competent retrovirus that does not require helper virus or additional nucleic acid sequence or proteins in order to propagate and produce virion. For example, the nucleic acid sequences of the retrovirus of the disclosure encode a group specific antigen and reverse transcriptase, (and integrase and protease-enzymes necessary for maturation and reverse transcription), respectively, as discussed above. The viral gag and pol can be derived from a lentivirus, such as HIV or an oncovirus or gammaretrovirus such as MoMLV. In addition, the nucleic acid genome of the retrovirus of the disclosure includes a sequence encoding a viral envelope (ENV) protein. The env gene can be derived from any retroviruses. The env may be an amphotropic envelope protein which allows transduction of cells of human and other species, or may be an ecotropic envelope protein, which is able to transduce only mouse and rat cells. Further, it may be desirable to target the recombinant virus by linkage of the envelope protein with an antibody or a particular ligand for targeting to a receptor of a particular cell-type. As mentioned above, retroviral vectors can be made target specific by inserting, for example, a glycolipid, or a protein. Targeting is often accomplished by using an antibody to target the retroviral vector to an antigen on a particular cell-type (e.g., a cell type found in a certain tissue, or a cancer cell type). Those of skill in the art will know of, or can readily ascertain without undue experimentation, specific methods to achieve delivery of a retroviral vector to a specific target. In one embodiment, the env gene is derived from a non-retrovirus (e.g., CMV or VSV). Examples of retroviral-derived env genes include, but are not limited to: Moloney murine leukemia virus (MoMuLV), Harvey murine sarcoma virus (HaMuSV), murine mammary tumor virus (MuMTV), gibbon ape leukemia virus (GaLV), human immunodeficiency virus (HIV) and Rous Sarcoma Virus (RSV). Other env genes such as Vesicular stomatitis virus (VSV) (Protein G), cytomegalovirus envelope (CMV), or influenza virus hemagglutinin (HA) can also be used.

In one embodiment, the retroviral genome is derived from an onco-retrovirus, and more particularly a mammalian onco-retrovirus. In a further embodiment, the retroviral genome is derived from a gamma retrovirus, and more particularly a mammalian gamma retrovirus. By “derived” is meant that the parent polynucleotide sequence is a wild-type oncovirus which has been modified by insertion or removal of naturally occurring sequences (e.g., insertion of an IRES, insertion of a heterologous polynucleotide encoding a polypeptide or inhibitory nucleic acid of interest, swapping of a more effective promoter from a different retrovirus or virus in place of the wild-type promoter and the like).

Unlike recombinant retroviruses produced by standard methods in the art that are defective and require assistance in order to produce infectious vector particles, the disclosure provides a retrovirus that is replication-competent.

In another embodiment, the disclosure provides retroviral vectors that can be targeted using regulatory sequences. Cell- or tissue-specific regulatory sequences (e.g., promoters) can be utilized to target expression of gene sequences in specific cell populations. Suitable mammalian and viral promoters for the disclosure are described elsewhere herein. Accordingly, in one embodiment, the disclosure provides a retrovirus having tissue-specific promoter elements at the 5′ end of the retroviral genome. Typically, the tissue-specific regulatory elements/sequences are in the U3 region of the LTR of the retroviral genome, including for example cell- or tissue-specific promoters and enhancers to neoplastic cells (e.g., tumor cell-specific enhancers and promoters), and inducible promoters (e.g., tetracycline).

In some circumstances, it may be desirable to regulate expression. For example, different viral promoters with varying strengths of activity may be utilized depending on the level of expression desired. In mammalian cells, the CMV immediate early promoter if often used to provide strong transcriptional activation. Modified versions of the CMV promoter that are less potent have also been used when reduced levels of expression of the transgene are desired. When expression of a transgene in hematopoietic cells is desired, retroviral promoters such as the LTRs from MLV or MMTV can be used. Other viral promoters that can be used include SV40, RSV LTR, HIV-1 and HIV-2 LTR, adenovirus promoters such as from the E1A, E2A, or MLP region, AAV LTR, cauliflower mosaic virus, HSV-TK, and avian sarcoma virus.

Similarly tissue specific or selective promoters may be used to effect transcription in specific tissues or cells so as to reduce potential toxicity or undesirable effects to non-targeted tissues. For example, promoters such as the PSA, probasin, prostatic acid phosphatase or prostate-specific glandular kallikrein (hK2) may be used to target gene expression in the prostate. The Whey accessory protein (WAP) may be used for breast tissue expression (Andres et al., PNAS 84:1299-1303, 1987). Other promoters/regulatory domains that can be used are set forth in Table 3.

“Tissue-specific regulatory elements” are regulatory elements (e.g., promoters) that are capable of driving transcription of a gene in one tissue while remaining largely “silent” in other tissue types. It will be understood, however, that tissue-specific promoters may have a detectable amount of “background” or “base” activity in those tissues where they are silent. The degree to which a promoter is selectively activated in a target tissue can be expressed as a selectivity ratio (activity in a target tissue/activity in a control tissue). In this regard, a tissue specific promoter useful in the practice of the disclosure typically has a selectivity ratio of greater than about 5. Preferably, the selectivity ratio is greater than about 15.

In certain indications, it may be desirable to activate transcription at specific times after administration of the recombinant replication competent retrovirus of the disclosure (RRCR). This may be done with promoters that are hormone or cytokine regulatable. For example in therapeutic applications where the indication is a gonadal tissue where specific steroids are produced or routed to, use of androgen or estrogen regulated promoters may be advantageous. Such promoters that are hormone regulatable include MMTV, MT-1, ecdysone and RuBisco. Other hormone regulated promoters such as those responsive to thyroid, pituitary and adrenal hormones may be used. Cytokine and inflammatory protein responsive promoters that could be used include K and T Kininogen (Kageyama et al., 1987), c-fos, TNF-alpha, C-reactive protein (Arcone et al., 1988), haptoglobin (Oliviero et al., 1987), serum amyloid A2, C/EBP alpha, IL-1, IL-6 (Poli and Cortese, 1989), Complement C3 (Wilson et al., 1990), IL-8, alpha-1 acid glycoprotein (Prowse and Baumann, 1988), alpha-1 antitypsin, lipoprotein lipase (Zechner et al., 1988), angiotensinogen (Ron et al., 1990), fibrinogen, c-jun (inducible by phorbol esters, TNF-alpha, UV radiation, retinoic acid, and hydrogen peroxide), collagenase (induced by phorbol esters and retinoic acid), metallothionein (heavy metal and glucocorticoid inducible), Stromelysin (inducible by phorbol ester, interleukin-1 and EGF), alpha-2 macroglobulin and alpha-1 antichymotrypsin. Tumor specific promoters such as osteocalcin, hypoxia-responsive element (HRE), MAGE-4, CEA, alpha-fetoprotein, GRP78/BiP and tyrosinase may also be used to regulate gene expression in tumor cells.

In addition, this list of promoters should not be construed to be exhaustive or limiting, those of skill in the art will know of other promoters that may be used in conjunction with the promoters and methods disclosed herein.

TABLE 3
TISSUE SPECIFIC PROMOTERS

| Tissue | Promoter |
|---|---|
| Pancreas | Insulin Elastin Amylase |
| | pdr-1 pdx-1 glucokinase |
| Liver | Albumin PEPCK HBV enhancer |
| | α fetoprotein apolipoprotein C α-1 |
| | antitrypsin vitellogenin, NF-AB |
| | Transthyretin |
| Skeletal muscle | Myosin H chain Muscle creatine kinase |
| | Dystrophin Calpain p94 Skeletal |
| | alpha-actin fast troponin 1 |
| Skin | Keratin K6 Keratin K1 |
| Lung | CFTR Human cytokeratin 18 (K18) |
| | Pulmonary surfactant proteins A, B |
| | and C CC-10 P1 |
| Smooth muscle | sm22 α SM-alpha-actin |
| Endothelium | Endothelin-1 E-selectin von |
| | Willebrand factor TIE (Korhonen et |
| | al., 1995) KDR/flk-1 Melanocytes |
| | Tyrosinase |
| Adipose tissue | Lipoprotein lipase (Zechner et al., |
| | 1988) Adipsin (Spiegelman et al., 1989) |
| | acetyl-CoA carboxylase (Pape and Kim, |
| | 1989) glycerophosphate dehydrogenase |
| | (Dani et al., 1989) adipocyte P2 (Hunt |
| | et al., 1986) |
| Breast | Whey Acidic Protien (WAP) (Andres et al. |
| | PNAS 84: 1299-1303 1987 |
| Blood | β-globin |

It will be further understood that certain promoters, while not restricted in activity to a single tissue type, may nevertheless show selectivity in that they may be active in one group of tissues, and less active or silent in another group. Such promoters are also termed “tissue specific”, and are contemplated for use with the disclosure. For example, promoters that are active in a variety of central nervous system (CNS) neurons may be therapeutically useful in protecting against damage due to stroke, which may affect any of a number of different regions of the brain. Accordingly, the tissue-specific regulatory elements used in the disclosure, have applicability to regulation of the heterologous proteins as well as an applicability as a targeting polynucleotide sequence in the present retroviral vectors.

In yet another embodiment, the disclosure provides plasmids comprising a recombinant retroviral derived construct. The plasmid can be directly introduced into a target cell or a cell culture such as NIH 3T3 or other tissue culture cells. The resulting cells release the retroviral vector into the culture medium.

In view of the foregoing, and the following example, the disclosure provides in one embodiment, a recombinant replication competent retrovirus (RCR) comprising an optimized IRES cassette. In one embodiment, the retroviral polynucleotide sequence is derived from a virus selected from the group consisting of murine leukemia virus (MLV), Moloney murine leukemia virus (MoMLV), Feline leukemia virus (FeLV), Baboon endogenous retrovirus (BEV), porcine endogenous virus (PERV), the cat derived retrovirus RD114, squirrel monkey retrovirus, Xenotropic murine leukemia virus-related virus (XMRV), avian reticuloendotheliosis virus (REV), or Gibbon ape leukemia virus (GALV). In another embodiment the RCR comprises a retroviral GAG protein; retroviral POL protein; a retroviral envelope (which can be chimeric, ecotropic and amphotropic); a retroviral polynucleotide comprising Long-Terminal Repeat (LTR) sequences at the 3′ end of the retroviral polynucleotide sequence, gag, pol and env genes and an optimized IRES cassette (and/or optional additional elements including core promoter, inhibitory nucleic acid such as miRNA and the like) and a promoter within the LTR at the 5′ end of the retroviral polynucleotide. In one embodiment, the 3′ LTR comprises a sequence that is at least 98% identical to the sequence from about nucleotide 9405 to about 9998 of SEQ ID NO:19, 22 or 42. In another embodiment, the promoter sequence at the 5′ end of the retroviral polynucleotide is suitable for expression in a mammalian cell. In another embodiment of any of the foregoing, the promoter, gag, pol and env domains comprise a sequence that is at least 98% identical to the sequence from about 1 to about 8323 of SEQ ID NO: 19, 22 or 42 and wherein the retroviral polynucleotide lacks 70 base pairs of MLV sequence downstream form the 3′LTR compared to a vector of SEQ ID NO:21 (pACE). In yet another embodiment of any of the foregoing, a cassette comprising an optimized internal ribosome entry site (IRES) comprising a sequence that is at least 98% identical to the sequence from about 8327 to 8875 of SEQ ID NO: 19, 22 or 42 and consisting of 5-6As in the A-bulge in the J-K bifurcation region. In a further embodiment, the optimized IRES is operably linked to a heterologous polynucleotide, wherein the cassette is positioned 5′ to the 3′ LTR and 3′ to the env nucleic acid domain encoding the retroviral envelope and lacking small repeats on either side of the cassette compared to the pACE vector of SEQ ID NO:21 (pACE-CD). In yet another embodiment of any of the foregoing, the vector includes cis-acting sequences necessary for reverse transcription, packaging and integration in a target cell. In still another embodiment, the RCR maintains higher replication competency after 6 passages compared to a vector comprising SEQ ID NO:21 (pACE) and wherein when the heterologous polynucleotide is expressed it produces at least 20%, 30%, 40%, 50% or more expressed heterologous polypeptide compared to a pAC3-yCD2 (SEQ ID NO:22) vector. In another embodiment, the RCR infects a target cell multiple times resulting in an average number of copies/diploid genome of 5 or greater. In another embodiment, the retroviral envelope is an amphotropic MLV envelope. In one embodiment, the promoter comprises a CMV promoter having a sequence as set forth in SEQ ID NO:19, 20, 22 or 42 from nucleotide 1 to about nucleotide 582 and may include modification to one or more nucleic acid bases and which is capable of directing and initiating transcription. In another embodiment, the promoter comprises a CMV-R-U5 domain polynucleotide. In still a further embodiment, the CMV-R-U5 domain comprises the immediately early promoter from human cytomegalovirus linked to an MLV R-U5 region. In yet a further embodiment, the CMV-R-U5 domain polynucleotide comprises a sequence as set forth in SEQ ID NO:19, 20, 22 or 42 from about nucleotide 1 to about nucleotide 1202 or sequences that are at least 99% identical to a sequence as set forth in SEQ ID NO:19, 20, 22 or 42, wherein the polynucleotide promotes transcription of a nucleic acid molecule operably linked thereto. In another embodiment, the gag nucleic acid domain comprises a sequence from about nucleotide number 1203 to about nucleotide 2819 of SEQ ID NO: 19, 22 or 42 or a sequence having at least 99% or 99.8% identity thereto. In another embodiment, embodiment, the pol domain of the polynucleotide is derived from a gammaretrovirus. In a further embodiment, the pol domain comprises a sequence from about nucleotide number 2820 to about nucleotide 6358 of SEQ ID NO: 19, 22 or 42 or a sequence having at least 99% or 99.9% identity thereto. In yet another embodiment, the env domain comprises a sequence from about nucleotide number 6359 to about nucleotide 8323 of SEQ ID NO: 19, 22 or 42 or a sequence having at least 99% or 99.8% identity thereto. In yet another embodiment, the IRES comprises a sequence as set forth in SEQ ID NO:41. In yet another embodiment, the heterologous nucleic acid comprises a polynucleotide having a sequence as set forth in SEQ ID NO:3, 5, 11, 13, 15 or 17. In another embodiment, the heterologous nucleic acid encodes a polypeptide comprising a sequence as set forth in SEQ ID NO:4. In a further embodiment, the heterologous nucleic acid is human codon optimized and encodes a polypeptide as set forth in SEQ ID NO:4. In yet another embodiment, the heterologous nucleic acid comprises a sequence as set forth in SEQ ID NO: 19, 22 or 42 from about nucleotide number 8877 to about 9353. In another embodiment, the 3′ LTR comprises a U3-R-U5 domain. In yet a further embodiment, the 3′ LTR comprises a sequence as set forth in SEQ ID NO: 19, 22 or 42 from about nucleotide 9405 to about 9998 or a sequence that is at least 95%, 98% or 99.5% identical thereto. In one embodiment, the disclosure provides a retroviral polynucleotide comprising SEQ ID NO:42. In another embodiment the retroviral polynucleotide of SEQ ID NO:42 is an RNA sequence wherein T is replaced with U. In yet another embodiment, a retroviral RNA polynucleotide according to SEQ ID NO:42, wherein T is U is encapsulated in a viral capsid. In yet another embodiment, of any of the foregoing, the retroviral polynucleotide can further comprise and miRNA, siRNA or shRNA sequence to be delivered to a target cell. The miRNA, siRNA or shRNA can be operably linked to a polIII promoter. The miRNA may be located upstream or downstream of the optimized IRES cassette. In another embodiment, the heterologous polynucleotide can be any number of coding sequences including cytokines, immunopotentiating agents, thymidine kinase, cytosine deaminase, purine nucleoside phophorylase, receptors, antibody and fragments etc.

The disclosure also provides a method of treating a cell proliferative disorder comprising contacting the subject with a retrovirus as described herein. In one embodiment, the retrovirus containing an optimized IRES under conditions such that a heterologous polynucleotide linked to the optimized IRES comprises cytosine deaminase activity and contacting the subject with 5-fluorocytosine. In one embodiment, the retrovirus infects a cell resulting in integration of a polynucleotide comprising SEQ ID NO:42. In another embodiment, the cell proliferative disorder is glioblastoma multiforme. In another embodiment, the cell proliferative disorder is selected from the group consisting of lung cancer, colon-rectum cancer, breast cancer, prostate cancer, urinary tract cancer, uterine cancer, brain cancer, head and neck cancer, pancreatic cancer, melanoma, stomach cancer and ovarian cancer. The method can include a combination therapy, wherein a subject to be treated is contacted with a retrovirus and further contacted with an anticancer agent or chemotherapeutic agent. For example, the anticancer or chemotherapeutic agent can be selected from the group consisting of bevacizumab, pegaptanib, ranibizumab, sorafenib, sunitinib, AE-941, VEGF Trap, pazopanib, vandetanib, vatalanib, cediranib, fenretinide, squalamine, INGN-241, oral tetrathiomolybdate, tetrathiomolybdate, Panzem NCD, 2-methoxyestradiol, AEE-788, AG-013958, bevasiranib sodium, AMG-706, axitinib, BIBF-1120, CDP-791, CP-547632, PI-88, SU-14813, SU-6668, XL-647, XL-999, IMC-1121B, ABT-869, BAY-57-9352, BAY-73-4506, BMS-582664, CEP-7055, CHIR-265, CT-322, CX-3542, E-7080, ENMD-1198, OSI-930, PTC-299, Sirna-027, TKI-258, Veglin, XL-184, or ZK-304709.

In another embodiment of any of the foregoing, a retrovirus is administered from about 10³ to 10⁷ TU/g brain weight. In another embodiment, the retrovirus is administered from about 10⁴ to 10⁶ TU/g brain weight.

The disclosure provides a polynucleotide construct comprising from 5′ to 3′: a promoter or regulatory region useful for initiating transcription; a psi packaging signal; a gag encoding nucleic acid sequence, a pol encoding nucleic acid sequence; an env encoding nucleic acid sequence; an internal ribosome entry site nucleic acid sequence comprising 5-6 A's in the A-bulge; a heterologous polynucleotide encoding a marker, therapeutic or diagnostic polypeptide; and a LTR nucleic acid sequence. As described elsewhere herein and as follows the various segment of the polynucleotide construct of the disclosure (e.g., a recombinant replication competent retroviral polynucleotide) are engineered depending in part upon the desired host cell, expression timing or amount, and the heterologous polynucleotide. A replication competent retroviral construct of the disclosure can be divided up into a number of domains that may be individually modified by those of skill in the art.

For example, the promoter can comprise a CMV promoter having a sequence as set forth in SEQ ID NO:19, 20, 22 or 42 from nucleotide 1 to about nucleotide 582 and may include modification to one or more (e.g., 2-5, 5-10, 10-20, 20-30, 30-50 or more nucleic acid bases) so long as the modified promoter is capable of directing and initiating transcription. In one embodiment, the promoter or regulatory region comprises a CMV-R-U5 domain polynucleotide. The CMV-R-U5 domain comprises the immediately early promoter from human cytomegalovirus to the MLV R-U5 region. In one embodiment, the CMV-R-U5 domain polynucleotide comprises a sequence as set forth in SEQ ID NO: 19, 20, 22 or 42 from about nucleotide 1 to about nucleotide 1202 or sequences that are at least 95% identical to a sequence as set forth in SEQ ID NO: 19, 20, 22 or 42 from about nucleotide 1 to about nucleotide 1202, wherein the polynucleotide promotes transcription of a nucleic acid molecule operably linked thereto. The gag domain of the polynucleotide may be derived from any number of retroviruses, but will typically be derived from an oncoretrovirus and more particularly from a mammalian oncoretrovirus. In one embodiment the gag domain comprises a sequence from about nucleotide number 1203 to about nucleotide 2819 of a sequence as set forth in SEQ ID NO: 19, 20, 22 or 42 or a sequence having at least 95%, 98%, 99% or 99.8% (rounded to the nearest 10^{th}) identity thereto. The poi domain of the polynucleotide may be derived from any number of retroviruses, but will typically be derived from an oncoretrovirus and more particularly from a mammalian oncoretrovirus. In one embodiment the pol domain comprises a sequence from about nucleotide number 2820 to about nucleotide 6358 of a sequence as set forth in SEQ ID NO: 19, 20, 22 or 42 or a sequence having at least 95%, 98%, 99% or 99.9% (roundest to the nearest 10^{th}) identity thereto. The env domain of the polynucleotide may be derived from any number of retroviruses, but will typically be derived from an oncoretrovirus or gamma-retrovirus and more particularly from a mammalian oncoretrovirus or gamma-retrovirus. In some embodiments the env coding domain comprises an amphotropic env domain. In one embodiment the env domain comprises a sequence from about nucleotide number 6359 to about nucleotide 8323 of a sequence as set forth in SEQ ID NO: 19, 20, 22 or 42 or a sequence having at least 95%, 98%, 99% or 99.8% (roundest to the nearest 10^{th}) identity thereto. The optimized IRES domain of the polynucleotide may be obtained from any number of internal ribosome entry sites. In one embodiment, optimized IRES is derived from an encephalomyocarditis virus. In one embodiment the optimized IRES domain comprises a sequence as set forth in SEQ ID NO:41 or a sequence having at least 95%, 98%, or 99% (roundest to the nearest 10^{th}) identity thereto so long as the domain allows for entry of a ribosome and comprises 5-6 A's in the A-bulge. The heterologous domain can comprise a cytosine deaminase (CD) of the disclosure. In one embodiment, the CD polynucleotide comprises a human codon optimized sequence. In yet another embodiment, the CD polynucleotide encodes a mutant polypeptide having cytosine deaminase, wherein the mutations confer increased thermal stabilization that increase the melting temperature (Tₘ) by 10° C. allowing sustained kinetic activity over a broader temperature range and increased accumulated levels of protein. In another embodiment, the disclosure comprises a human codon optimized thymidine kinase. The heterologous domain may be followed by a polypurine rich domain. The 3′ LTR can be derived from any number of retroviruses, typically an oncoretrovirus and preferably a mammalian oncoretrovirus. In one embodiment, the 3′ LTR comprises a U3-R-U5 domain. In yet another embodiment the LTR comprises a sequence as set forth in SEQ ID NO:19, 20, 22 or 42 from about nucleotide 9405 to about 9998 or a sequence that is at least 95%, 98% or 99.5% (rounded to the nearest 10^{th}) identical thereto.

The disclosure also provides a recombinant retroviral vector comprising from 5′ to 3′ a CMV-R-U5, fusion of the immediate early promoter from human cytomegalovirus to the MLV R-U5 region; a PBS, primer binding site for reverse transcriptase; a 5′ splice site; a ψ packaging signal; a gag, ORF for MLV group specific antigen; a pol, ORF for MLV polymerase polyprotein; a 3′ splice site; a 4070A env, ORF for envelope protein of MLV strain 4070A; an optimized IRES, consisting of 5-6A's in the A-bulge; a modified cytosine deaminase (thermostabilized and codon optimized) or human codon optimized thymidine kinase; a PPT, polypurine tract; and a U3-R-U5, MLV long terminal repeat.

The disclosure also provides a retroviral vector comprising a sequence as set forth in SEQ ID NO:42 (or SEQ ID NO:42 wherein T can be U) comprising an optimized A-bulge for expression. In one embodiment, the optimized A-bulge of the IRES consists of 5-6A's.

The retroviral vectors can be used to treat a wide range of disease and disorders including a number of cell proliferative diseases and disorders (see, e.g., U.S. Pat. Nos. 4,405,712 and 4,650,764; Friedmann, 1989, Science, 244:1275-1281; Mulligan, 1993, Science, 260:926-932, R. Crystal, 1995, Science 270:404-410, each of which are incorporated herein by reference in their entirety, see also, The Development of Human Gene Therapy, Theodore Friedmann, Ed., Cold Spring Harbor Laboratory Press, Cold Spring Harbor, N.Y., 1999. ISBN 0-87969-528-5, which is incorporated herein by reference in its entirety).

The disclosure also provides gene therapy for the treatment of cell proliferative disorders. Such therapy would achieve its therapeutic effect by introduction of an appropriate therapeutic polynucleotide (e.g., antisense, ribozymes, suicide genes, siRNA), into cells of subject having the proliferative disorder. Delivery of polynucleotide constructs can be achieved using the recombinant retroviral vector of the disclosure, particularly if it is based on MLV, which is capable of infecting dividing cells.

In addition, the therapeutic methods (e.g., the gene therapy or gene delivery methods) as described herein can be performed in vivo or ex vivo. It may be preferable to remove the majority of a tumor prior to gene therapy, for example surgically or by radiation. In some aspects, the retroviral therapy may be preceded or followed by surgery, chemotherapy or radiation therapy.

Thus, the disclosure provides a recombinant retrovirus capable of infecting a non-dividing cell, a dividing cell or a neoplastic cell, therein the recombinant retrovirus comprises a viral GAG; a viral POL; a viral ENV; a heterologous nucleic acid operably linked to an IRES consisting of 5-6A's in the A-bulge; and cis-acting nucleic acid sequences necessary for packaging, reverse transcription and integration. The recombinant retrovirus can be a lentivirus, such as HIV, or can be an oncovirus. As described above for the method of producing a recombinant retrovirus, the recombinant retrovirus of the disclosure may further include at least one of VPR, VIF, NEF, VPX, TAT, REV, and VPU protein. While not wanting to be bound by a particular theory, it is believed that one or more of these genes/protein products are important for increasing the viral titer of the recombinant retrovirus produced (e.g., NEF) or may be necessary for infection and packaging of virion.

The disclosure also provides a method of nucleic acid transfer to a target cell to provide expression of a particular nucleic acid (e.g., a heterologous sequence). Therefore, in another embodiment, the disclosure provides a method for introduction and expression of a heterologous nucleic acid in a target cell comprising infecting the target cell with the recombinant virus of the disclosure and expressing the heterologous nucleic acid in the target cell. As mentioned above, the target cell can be any cell type including dividing, non-dividing, neoplastic, immortalized, modified and other cell types recognized by those of skill in the art, so long as they are capable of infection by a retrovirus.

It may be desirable to modulate the expression of a gene in a cell by the introduction of a nucleic acid sequence (e.g., the heterologous nucleic acid sequence) by the method of the disclosure, wherein the nucleic acid sequence give rise, for example, to an antisense or ribozyme molecule. The term “modulate” envisions the suppression of expression of a gene when it is over-expressed, or augmentation of expression when it is under-expressed. Where a cell proliferative disorder is associated with the expression of a gene, nucleic acid sequences that interfere with the gene's expression at the translational level can be used. This approach utilizes, for example, antisense nucleic acid, ribozymes, or triplex agents to block transcription or translation of a specific mRNA, either by masking that mRNA with an antisense nucleic acid or triplex agent, or by cleaving it with a ribozyme.

It may be desirable to transfer a nucleic acid encoding a biological response modifier (e.g., a cytokine) into a cell or subject. Included in this category are immunopotentiating agents including nucleic acids encoding a number of the cytokines classified as “interleukins”. These include, for example, interleukins 1 through 15, as well as other response modifiers and factors described elsewhere herein. Also included in this category, although not necessarily working according to the same mechanisms, are interferons, and in particular gamma interferon, tumor necrosis factor (TNF) and granulocyte-macrophage-colony stimulating factor (GM-CSF). Other polypeptides include, for example, angiogenic factors and anti-angiogenic factors. It may be desirable to deliver such nucleic acids to bone marrow cells or macrophages to treat enzymatic deficiencies or immune defects. Nucleic acids encoding growth factors, toxic peptides, ligands, receptors, or other physiologically important proteins can also be introduced into specific target cells.

The disclosure can be used for delivery of heterologous polynucleotides that promote drug specific targeting and effects. For example, HER2, a member of the EGF receptor family, is the target for binding of the drug trastuzumab (Herceptin™, Genentech). Trastuzumab is a mediator of antibody-dependent cellular cytotoxicity (ADCC). Activity is preferentially targeted to HER2-expressing cells with 2+ and 3+ levels of overexpression by immunohistochemistry rather than 1+ and non-expressing cells (Herceptin prescribing information, Crommelin 2002). Enhancement of expression of HER2 by introduction of vector expressing HER2 or truncated HER2 (expressing only the extracellular and transmembrane domains) in HER2 low tumors may facilitate optimal triggering of ADCC and overcome the rapidly developing resistance to Herceptin that is observed in clinical use.

The substitution of yCD2 (comprising SEQ ID NO:19 from about 8877 to 9353) for the intracellular domain of HER2 allows for cell surface expression of HER2 and cytosolic localization of yCD2. The HER2 extracellular domain (ECD) and transmembrane domain (TM) (approximately 2026 bp from about position 175 to 2200 of SEQ ID NO:23) can be amplified by PCR (Yamamoto et al., Nature 319:230-234, 1986; Chen et al., Canc. Res., 58:1965-1971, 1998) or chemically synthesized (BioBasic Inc., Markham, Ontario, Canada) and inserted between the IRES and yCD2 gene in the vector pAC3-yCD2 SEQ ID NO: 19 (e.g., between about nucleotide 8876 and 8877 of SEQ ID NO:19). Alternatively, the yCD gene can be excised and replaced with a polynucleotide encoding a HER2 polypeptide or fragment thereof. A further truncated HER2 with only the Herceptin binding domain IV of the ECD and TM domains (approximately 290 bp from position 1910 to 2200) can be amplified or chemically synthesized and used as above (Landgraf 2007; Garrett et al., J. of Immunol., 178:7120-7131, 2007). A further modification of this truncated form with the native signal peptide (approximately 69 bp from position 175-237) fused to domain IV and the TM can be chemically synthesized and used as above. The resulting viruses can be used to treat a cell proliferative disorder in a subject in combination with trastuzumab or trastuzumab and 5-FC.

Alternatively, HER2 and the modifications described above can be expressed in a separate vector containing a different ENV gene or other appropriate surface protein. This vector can be replication competent (Logg et al. J. Mol Biol. 369:1214 2007) or non-replicative “first generation” retroviral vector that encodes the envelope and the gene of interest (Emi et al. J. Virol 65:1202 1991). In the latter case the pre-existing viral infection will provide complementary gag and pol to allow infective spread of the “non-replicative” vector from any previously infected cell. Alternate ENV and glycoproteins include xenotropic and polytropic ENV and glycoproteins capable of infecting human cells, for example ENV sequences from the NZB strain of MLV and glycoproteins from MCF, VSV, GALV and other viruses (Palu 2000, Baum et al., Mol. Therapy, 13(6):1050-1063, 2006). For example, a polynucleotide can comprise a sequence wherein the GAG and POL and yCD2 genes of SEQ ID NO: 19 are deleted, the ENV corresponds to a xenotropic ENV domain of NZB MLV or VSV-g, and the IRES or a promoter such as RSV is operatively linked directly to HER2, HER2 ECDTM, HER2 ECDIVTM, or HER2 SECDIVTM.

Mixed infection of cells by VSVG pseudotyped virus and amphotropic retrovirus results in the production of progeny virions bearing the genome of one virus encapsidated by the envelope proteins of the other. The same is true for other envelopes that pseudotype retroviral particles. For example, infection by retroviruses derived as above results in production of progeny virions capable of encoding yCD2 and HER2 (or variant) in infected cells. The resulting viruses can be used to treat a cell proliferative disorder in a subject in combination with trastuzumab or trastuzumab and 5-FC.

Another aspect of the development of resistance to trastuzumab relates to the interference with intracellular signaling required for the activity of trastuzumab. Resistant cells show loss of PTEN and lower expression of p27kip1 [Fujita, Brit J. Cancer, 94:247, 2006; Lu et al., Journal of the National Cancer Institute, 93(24): 1852-1857, 2001; Kute et al., Cytometry Part A 57A:86-93, 2004). For example, a polynucleotide encoding PTEN can be recombinantly generated or chemically synthesized (BioBasic Inc., Markham, Canada) and operably inserted directly after the yCD2 polynucleotide in the vector pAC3-yCD2 SEQ ID NO: 19 or 22, or with a linker sequence as previously described, or as a replacement for yCD2. In a further example, the PTEN encoding polynucleotide (SEQ ID NO:25) can be synthesized as above and inserted between the IRES and yCD2 sequences or with a linker as previously described.

Alternatively, PTEN can be expressed in a separate vector containing a different ENV gene or other appropriate surface protein. This vector can be replication competent (Logg et al. J. Mol Biol. 369:1214 2007) or non-replicative “first generation” retroviral vector that encodes the envelope and the gene of interest (Emi et al., J. Virol 65:1202 1991). In the latter case the pre-existing viral infection will provide complementary gag and pol to allow infective spread of the “non-replicative” vector from any previously infected cell. Alternate ENV and glycoproteins include xenotropic and polytropic ENV and glycoproteins capable of infecting human cells, for example ENV sequences from the NZB strain of MLV and glycoproteins from MCF, VSV, GALV and other viruses (Palu, Rev Med Virol. 2000, Baum, Mol. Ther. 13(6):1050-1063, 2006). For example, a polynucleotide can comprise a sequence wherein the GAG and POL and yCD2 genes of SEQ ID NO: 19 are deleted, the ENV corresponds to a xenotropic ENV domain of NZB MLV or VSV-g, and the IRES or a promoter such as RSV is operatively linked directly to PTEN.

Mixed infection of cells by VSVG pseudotyped virus and amphotropic retrovirus results in the production of progeny virions bearing the genome of one virus encapsidated by the envelope proteins of the other [Emi 1991]. The same is true for other envelopes that pseudotype retroviral particles. For example, infection by retroviruses derived as above results in production of progeny virions capable of encoding yCD2 and PTEN (or variant) or PTEN alone in infected cells. The resulting viruses can be used to treat a cell proliferative disorder in a subject in combination with trastuzumab or trastuzumab and 5-FC.

Similarly, a polynucleotide encoding p27kip1 (SEQ ID NO:27 and 28) can be chemically synthesized (BioBasic Inc., Markham, Canada) and operably inserted directly after the yCD2 gene in the vector pAC3-yCD2 SEQ ID NO:19 or SEQ ID NO:42 or with a linker sequence. In a further example, the p27kip1 encoding polynucleotide can be synthesized as above and inserted between the IRES consisting of 5-6A's in the A-bulge and yCD2 sequences or with a linker as previously described or in place of the yCD2 gene.

Alternatively, p27kip1 can be expressed in a separate vector containing a different ENV gene or other appropriate surface protein. This vector can be replication competent (Logg et al. J. Mol Biol. 369:1214 2007) or non-replicative “first generation” retroviral vector that encodes the envelope and the gene of interest (Emi et al. J. Virol 65:1202 1991). In the latter case the pre-existing viral infection will provide complementary gag and pol to allow infective spread of the “non-replicative” vector from any previously infected cell. Alternate ENV and glycoproteins include xenotropic and polytropic ENV and glycoproteins capable of infecting human cells, for example ENV sequences from the NZB strain of MLV and glycoproteins from MCF, VSV, GALV and other viruses (Palu 2000, Baum 2006, supra). For example, a polynucleotide can comprise a sequence wherein the GAG and POL and yCD2 genes of SEQ ID NO: 19 are deleted, the ENV corresponds to a xenotropic ENV domain of NZB MLV or VSV-g, and the IRES consisting of 5-6A's in the A-bulge or a promoter such as RSV is operatively linked directly to p27kip1.

Mixed infection of cells by VSVG pseudotyped virus and amphotropic retrovirus results in the production of progeny virions bearing the genome of one virus encapsidated by the envelope proteins of the other [Emi 1991]. The same is true for other envelopes that pseudotype retroviral particles. For example, infection by retroviruses derived as above from both SEQ ID NO:19, 22 and 42 results in production of progeny virions capable of encoding yCD2 and p27kip1 (or variant) in infected cells. The resulting viruses can be used to treat a cell proliferative disorder in a subject in combination with trastuzumab or trastuzumab and 5-FC.

In another example, CD20 is the target for binding of the drug rituximab (Rituxan™, Genentech). Rituximab is a mediator of complement-dependent cytotoxicity (CDC) and ADCC. Cells with higher mean fluorescence intensity by flow cytometry show enhanced sensitivity to rituximab (van Meerten et al., Clin Cancer Res 2006; 12(13):4027-4035, 2006). Enhancement of expression of CD20 bp introduction of vector expressing CD20 in CD20 low B cells may facilitate optimal triggering of ADCC.

For example, a polynucleotide encoding CD20 (SEQ ID NO:29 and 30) can be chemically synthesized (BioBasic Inc., Markham, Canada) and operably inserted directly after the yCD2 gene in the vector pAC3-yCD2 (-2) SEQ ID NO: 19, 22 or 42 with a linker sequence as previously described, or as a replacement for the yCD2 gene. In a further example, the CD20 encoding polynucleotide can be synthesized as above and inserted between the IRES consisting of 5-6A's in the A-bulge and yCD2 sequences or with a linker as previously described. As a further alternative the CD20 sequence can be inserted into the pAC3-yCD2 vector after excision of the CD gene by Psi1 and Not1 digestion.

In still a further example, a polynucleotide encoding CD20 (SEQ ID NO:29 and 30) can be chemically synthesized (BioBasic Inc., Markham, Canada)and inserted into a vector containing a non amphotropic ENV gene or other appropriate surface protein (Tedder et al., PNAS, 85:208-212, 1988). Alternate ENV and glycoproteins include xenotropic and polytropic ENV and glycoproteins capable of infecting human cells, for example ENV sequences from the NZB strain of MLV and glycoproteins from MCF, VSV, GALV and other viruses [Palu 2000, Baum 2006]. For example, a polynucleotide can comprise a sequence wherein the GAG and POL and yCD2 genes of SEQ ID NO: 19 are deleted, the ENV corresponds to a xenotropic ENV domain of NZB MLV or VSV-g, and the IRES consisting of 5-6A's in the A-bulge or a promoter such as RSV is operatively linked directly to CD20.

Mixed infection of cells by VSVG pseudotyped virus and amphotropic retrovirus results in the production of progeny virions bearing the genome of one virus encapsidated by the envelope proteins of the other (Emi 1991). The same is true for other envelopes that pseudotype retroviral particles. For example, infection by retroviruses derived as above from SEQ ID NO:19, 22 or 42 results in production of progeny virions capable of encoding yCD2 and CD20 in infected cells. The resulting viruses can be used to treat a cell proliferative disorder in a subject in combination with Rituxan and/or 5-FC. Similarly, infection of a tumor with a vector encoding only the CD20 marker can make the tumor treatable by the use of Rituxan.

Levels of the enzymes and cofactors involved in pyrimidine anabolism can be limiting. OPRT, thymidine kinase (TK), Uridine monophosphate kinase, and pyrimidine nucleoside phosphorylase expression is low in 5-FU resistant cancer cells compared to sensitive lines (Wang et al., Cancer Res., 64:8167-8176, 2004). Large population analyses show correlation of enzyme levels with disease outcome (Fukui et al., Int'l. J. OF Mol. Med., 22:709-716, 2008). Coexpression of CD and other pyrimidine anabolism enzymes (PAE) can be exploited to increase the activity and therefore therapeutic index of fluoropyrimidine drugs.

The disclosure provides methods for treating cell proliferative disorders such as cancer and neoplasms comprising administering an RCR vector of the disclosure followed by treatment with a chemotherapeutic agent or anti-cancer agent. In one aspect, the RCR vector is administered to a subject for a period of time prior to administration of the chemotherapeutic or anti-cancer agent that allows the RCR to infect and replicate. The subject is then treated with a chemotherapeutic agent or anti-cancer agent for a period of time and dosage to reduce proliferation or kill the cancer cells. In one aspect, if the treatment with the chemotherapeutic or anti-cancer agent reduces, but does not kill the cancer/tumor (e.g., partial remission or temporary remission), the subject may then be treated with a non-toxic therapeutic agent (e.g., 5-FC) that is converted to a toxic therapeutic agent in cells expression a cytotoxic gene (e.g., cytosine deaminase) from the RCR.

Using such methods the RCR vectors of the disclosure are spread during a replication process of the tumor cells, such cells can then be killed by treatment with an anti-cancer or chemotherapeutic agent and further killing can occur using the RCR treatment process described herein.

In yet another embodiment of the disclosure, the heterologous gene can comprise a coding sequence for a target antigen (e.g., a cancer antigen). In this embodiment, cells comprising a cell proliferative disorder are infected with an RCR comprising a heterologous polynucleotide encoding the target antigen to provide expression of the target antigen (e.g., overexpression of a cancer antigen). An anticancer agent comprising a targeting cognate moiety that specifically interacts with the target antigen is then administered to the subject. The targeting cognate moiety can be operably linked to a cytotoxic agent or can itself be an anticancer agent. Thus, a cancer cell infected by the RCR comprising the targeting antigen coding sequences increases the expression of target on the cancer cell resulting in increased efficiency/efficacy of cytotoxic targeting.

In yet another embodiment, an RCR of the disclosure can comprise a coding sequence comprising a binding domain (e.g., an antibody, antibody fragment, antibody domain or receptor ligand) that specifically interacts with a cognate antigen or ligand. The RCR comprising the coding sequence for the binding domain can then be used to infect cells in a subject comprising a cell proliferative disorder such as a cancer cell or neoplastic cell. The infected cell will then express the binding domain or antibody. An antigen or cognate operably linked to a cytotoxic agent or which is cytotoxic itself can then be administered to a subject. The cytotoxic cognate will then selectively kill infected cells expressing the binding domain. Alternatively the binding domain itself can be an anti-cancer agent.

As used herein, the term “antibody” refers to a protein that includes at least one immunoglobulin variable domain or immunoglobulin variable domain sequence. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody” encompasses antigen-binding fragments of antibodies (e.g., single chain antibodies, Fab fragments, F(ab′)2, a Fd fragment, a Fv fragments, and dAb fragments) as well as complete antibodies.

The disclosure provides a method of treating a subject having a cell proliferative disorder. The subject can be any mammal, and is preferably a human. The subject is contacted with a recombinant replication competent retroviral vector of the disclosure. The contacting can be in vivo or ex vivo. Methods of administering the retroviral vector of the disclosure are known in the art and include, for example, systemic administration, topical administration, intraperitoneal administration, intra-muscular administration, intracranial, cerebrospinal, as well as administration directly at the site of a tumor or cell-proliferative disorder. Other routes of administration known in the art.

Thus, the disclosure includes various pharmaceutical compositions useful for treating a cell proliferative disorder. The pharmaceutical compositions according to the disclosure are prepared by bringing a retroviral vector containing a heterologous polynucleotide sequence useful in treating or modulating a cell proliferative disorder according to the disclosure into a form suitable for administration to a subject using carriers, excipients and additives or auxiliaries. Frequently used carriers or auxiliaries include magnesium carbonate, titanium dioxide, lactose, mannitol and other sugars, talc, milk protein, gelatin, starch, vitamins, cellulose and its derivatives, animal and vegetable oils, polyethylene glycols and solvents, such as sterile water, alcohols, glycerol and polyhydric alcohols. Intravenous vehicles include fluid and nutrient replenishers. Preservatives include antimicrobial, anti-oxidants, chelating agents and inert gases. Other pharmaceutically acceptable carriers include aqueous solutions, non-toxic excipients, including salts, preservatives, buffers and the like, as described, for instance, in Remington's Pharmaceutical Sciences, 15th ed. Easton: Mack Publishing Co., 1405-1412, 1461-1487 (1975) and The National Formulary XIV., 14th ed. Washington: American Pharmaceutical Association (1975), the contents of which are hereby incorporated by reference. The pH and exact concentration of the various components of the pharmaceutical composition are adjusted according to routine skills in the art. See Goodman and Gilman's The Pharmacological Basis for Therapeutics (7th ed.).

For example, and not by way of limitation, a retroviral vector useful in treating a cell proliferative disorder will include an amphotropic ENV protein, GAG, and POL proteins, a promoter sequence in the U3 region retroviral genome, and all cis-acting sequence necessary for replication, packaging and integration of the retroviral genome into the target cell.

The following Examples are intended to illustrate, but not to limit the disclosure. While such Examples are typical of those that might be used, other procedures known to those skilled in the art may alternatively be utilized.

### EXAMPLES

### Example 1

The expression level of yCD2 and the conversion of 5-FC to 5-FU by yCD2 have been demonstrated to be efficient and stable both in vitro and in vivo when cells are maximally infected with Toca 511 (pAC3-yCD2; SEQ ID NO:22). However, in an in vivo pilot study in long-term (180 days approximately) infected Balb/c mice integrated proviruses from some tissues were shown to carry expanded or contracted oligo A sequences in the J-K bifurcation loop. In tissues from four mice of a biolocalization study analyzed by molecular PCR cloning, a heterogeneous expansion of 7A to 8A, 9A, 10A, 11A and 12A and a contraction of 7A to 6A was observed. This observation and the 7As in pEMCF as opposed to the 6As in ECMV IRES originally described, led to the investigation of the impact of the yCD2 expression mediated by IRES with various numbers of As in the A bulge, and, in particular, the impact on protein translation in the context of RRV. Accordingly, a series of deletion and insertion mutants specifically in the A bulge in the bifurcation region were generated. The data show that neither deletion nor insertion of the oligo A sequence in the A bulge affects RRV production, that 6 As provide maximal CD and green fluorescent protein (GFP) expression and that small changes in the number of As from the 6As have moderate effect, but that larger changes have drastic effects on efficiency of the IRES-mediated translation of mRNA from the transgene.

Construction of RRVs containing various numbers of A's in the A bulge of the J-K bifurcation region. RRVs containing an EMCV IRES and encoding CD or GFP were generated to have 4, 5, 6, 7, 8, 10 or 12As in the A-bulge in the J-K bifurcation region. Each construct was generated by DNA synthesis (BioBasics Inc.) of the entire IRES cassette with a Mlu I at the 5′ end and a Psi I at the 3′end, respectively, for direct replacement of the equivalent cassette in the RRV backbone (FIG. 1B). All DNA fragments were confirmed by sequencing analysis prior and post cloning into the RRV backbone. The RRV constructs containing the yCD2 transgene were designated using the name of the transgene followed by the number of A's in the A bulge (e.g., yCD2-4A contains yCD2 transgene and 4As in the A bulge in the IRES).

RRVs containing various numbers of A's in the A bulge produce similar titers. Virus stock was produced by transient transfection of 293T cells using calcium phosphate precipitation method. Viral supernatant was collected approximately 42 hours post transfection. Viral infection to determine titers was performed. Viral supernatant of each vector was subsequently used to infect HT1080 cells to generate RRV-producer cells. The viral titers obtained were measured before infecting naive U87-MG cells. FIG. 1C shows that HT1080 cells infected with RRVs containing various numbers of As produced similar levels of virus, suggesting that the number of the As in the bifurcation loop does not affect viral replication.

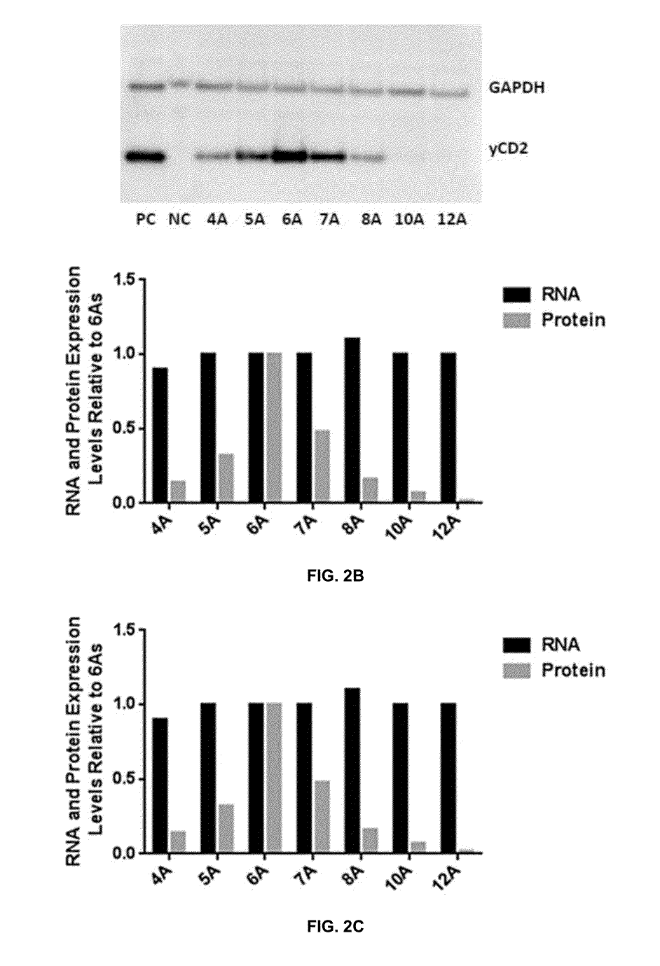

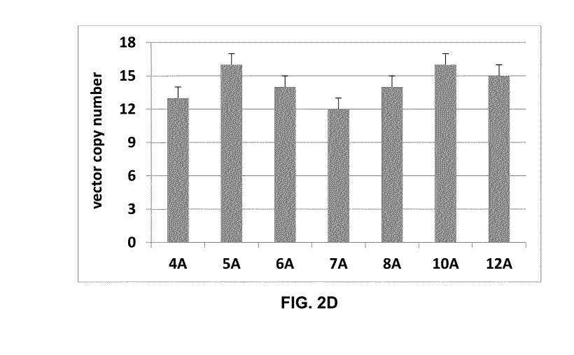

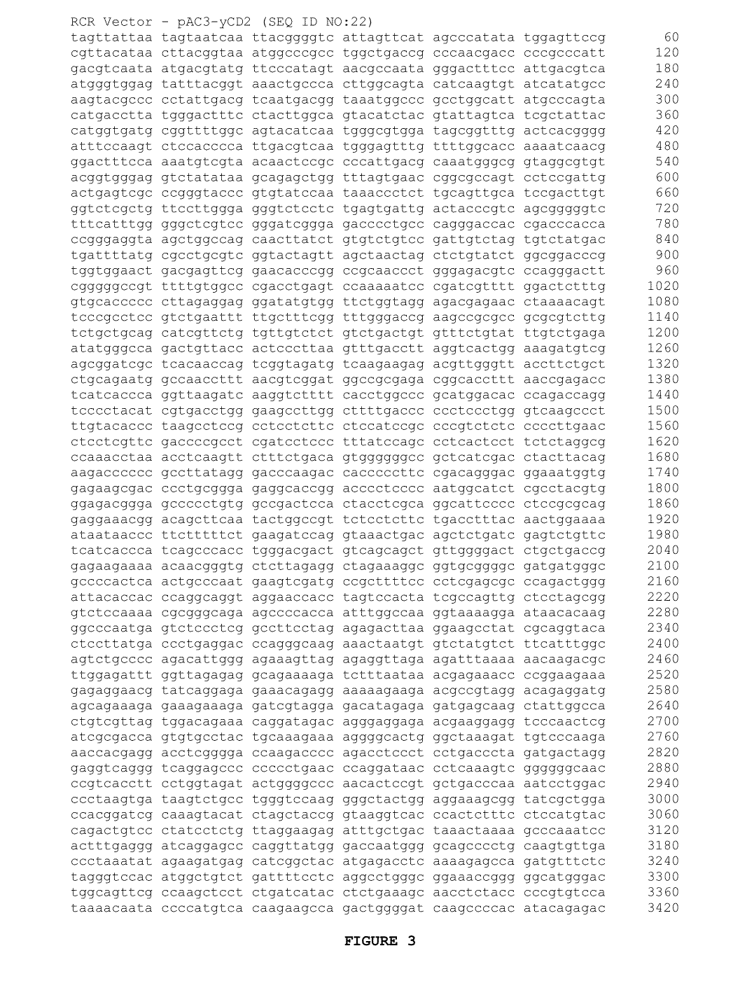

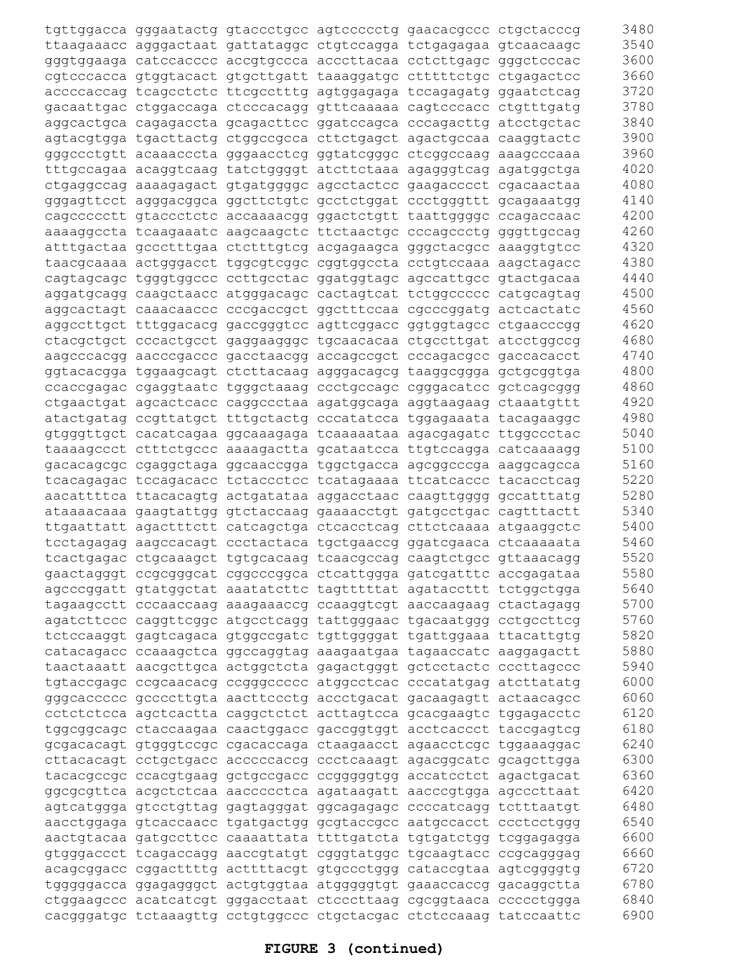

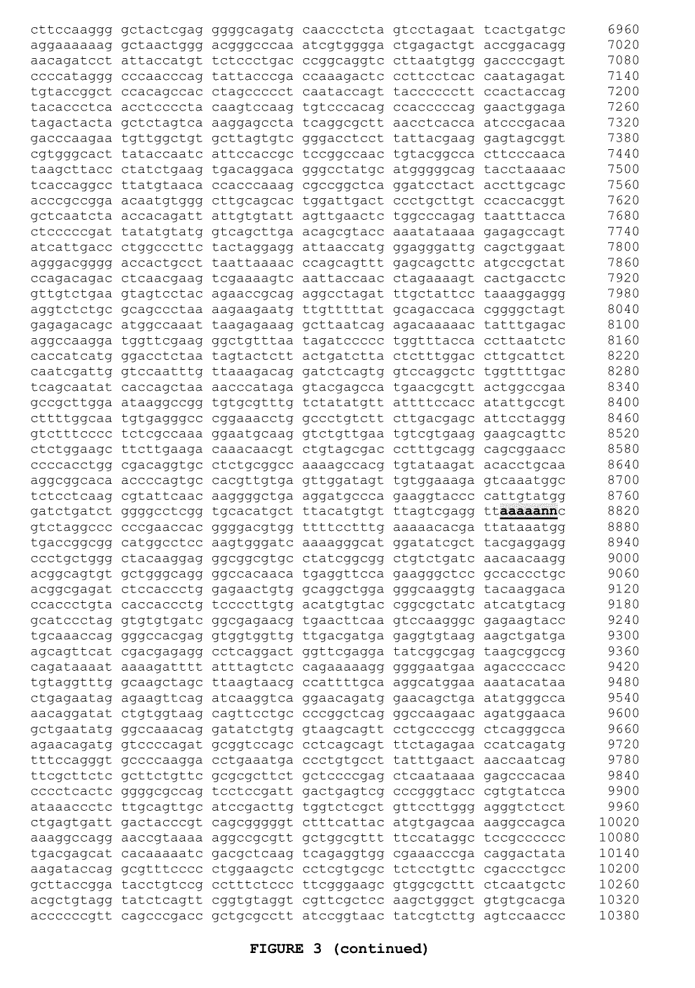

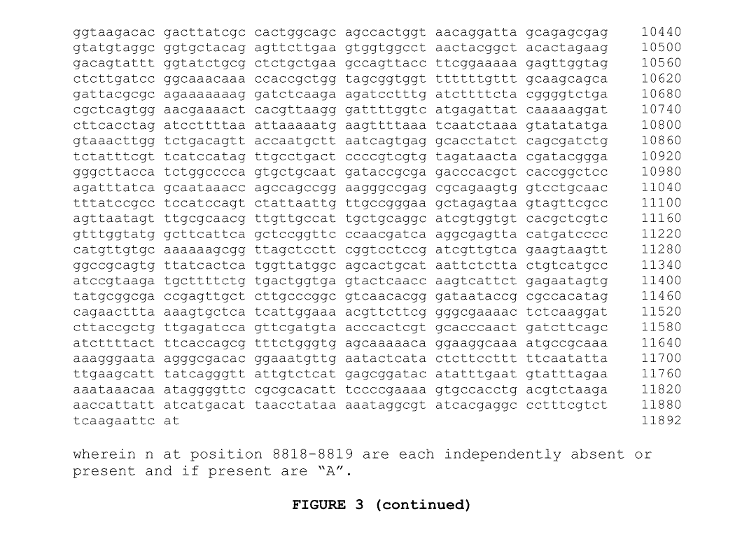

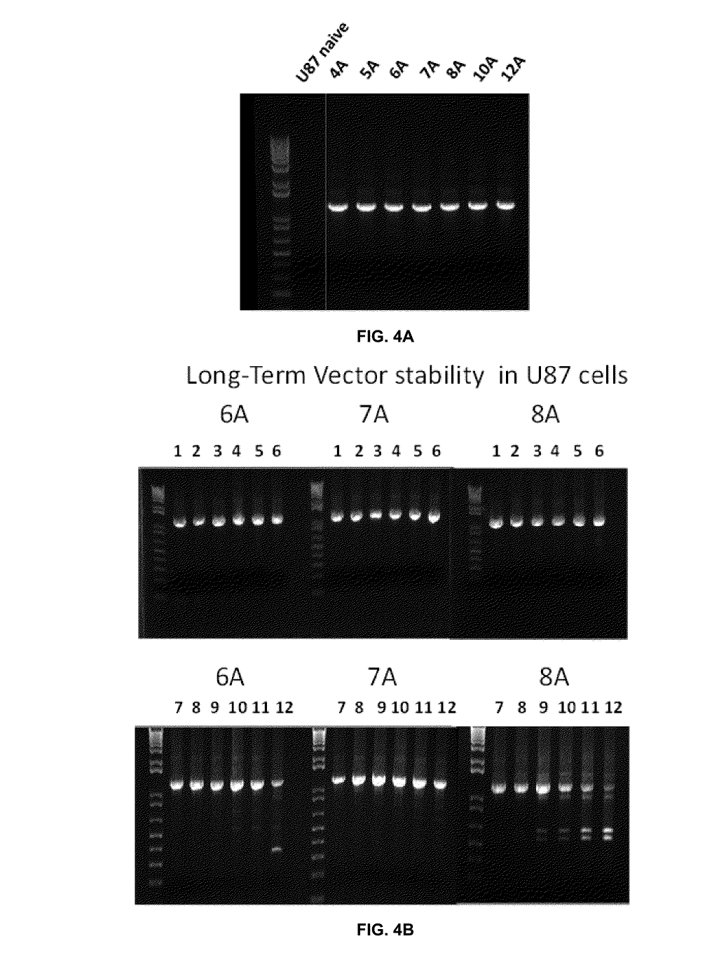

RRVs containing various numbers of A's in the J-K bifurcation region express similar levels of transcripts but different levels of protein expression. The viral supernatant from HT1080 cells was then used to infect naive U87-MG cells at multiplicity of infection (MOI) of 0.1. At day 10 post infection, when the cells were fully infected, cellular viral RNA levels were measured by quantitative real-time polymerase chain reaction (qRT-PCR), and protein expression level of yCD2 was examined by immunoblotting (Perez et al., 2012). The cellular viral RNA expression levels were measured using two different primer sets, located in the env (5′Env2: 5′-ACCCTCAACCTCCCCTACAAGT-3′ (SEQ ID NO:47), 3′Env2: 5′-GTTAAGCGCCTGATAGGCTC-3′ (SEQ ID NO:48), probe: 5′FAM-AGCCACCCCCAGGAACTGGAGATAGA-3′BHQ (SEQ ID NO:49)) and in yCD2 region (5′yCD2: 5′-ATCATCATGTACGGCATCCCTAG-3′ (SEQ ID NO:50), 3′yCD2: 5′-TGAACTGCTTCATCAGCTTCTTAC-3′ (SEQ ID NO:51), probe: 5′FAM-TCATCGTCAACAACCACCACCTCGT-3′BHQ (SEQ ID NO:52)), respectively, (FIG. 2). The relative level of RNA from each vector was calculated using 2-^{ΔΔ(Ct)} method with respect to the vector containing the 6As. The cellular viral RNA level ratios range from 0.8 to 1.1 (FIG. 2), suggesting that there is no significant difference in viral RNA transcript due to modifications in the IRES. In examining the yCD2 protein expression level of these vectors by Western blot, yCD2 protein expression levels of the vectors containing the 5 and 7As were identified as being 69% and 77% that of the yCD2-6A vector. In contrast, a substantial reduction of yCD2 protein expression was observed in the vectors containing the 4, 8, 10 and 12As. The CD protein expression levels of these vectors range from 4 to 25% that of the yCD2-6A vector (FIG. 2B). The drastic reduction of the yCD2 protein expression with similar expression levels of the cellular viral RNA suggested that the length of oligo A in the bifurcation region in the IRES can have a large effect on gene expression at the post-transcriptional level. Relative intracellular CD enzymatic activity was also measure by adding 5 FC to the cultures and measuring 5-FU after an hour. The differences in activity were ranked similarly to the Western blot data, but were not as marked. This can be attributed to limitations in a cell-based assay and to the low availability of intracellular 5-FC which was below the Kₘ for the enzyme in the assay utilized. Therefore, the effect of the number of A's in the loop were analyzed with another transgene for which the protein expression assay was well defined. Also, using a different transgene would allow a determination of whether or not the alteration in yCD2 protein expression with change in number of A's in the A bulge is transgene-specific.

An equivalent set of RRVs encoding GFP were generated. The viral titers of these vectors were also comparable to one another and this data looked very similar to that with the yCD2 transgene (FIG. 1C). The GFP expression levels were measured using flow cytometry by gating the GFP-positive cells. The mean fluorescent intensity (MFI) of each vector was normalized to the cellular viral RNA level and calculated relative to the GFP-6A vector. The results (FIG. 2C), from this set of vector were consistent with those observed with yCD2 vectors (FIG. 2B) and the vectors containing the 6As expresses the highest level of protein from the transgene in both sets of vectors. Furthermore, due to the sensitivity of the detection method, a remarkable difference in GFP expression level was revealed, showing approximately 96% and 99% decrease in GFP expressed by the vectors containing the 10As and 12As, respectively. In both sets of the vectors, RRV with 7As showed an approximately 30% decrease in protein expression. Consistent with findings reported by Hoffman et al., RRV with 4As and 5As, respectively, showed similar phenotype as 868Δ4 described by Hoffman et al. with markedly reduced protein translation efficiency compared to RRV with 6As.

The disclosure demonstrates that the length of the A-bulge in the J-K bifurcation region affects expression of the transgene downstream of the IRES presumably through effects on the translation efficiency. Previous findings implying that the context around AUG11, the spacing between the polypyrimidine tract located in the 3′ IRES and the first AUG in the cistron as well as the arrangement of cistron on the mRNA all play a role in modulating protein translation. The data show that the presence of 6 As provides the highest level of transgene protein expression and alteration of the numbers of As in the A bulge by contraction or expansion of 2-4 nucleotides could significantly affect the expression level of the transgene downstream of the IRES. The protein expression results suggest that the optimum IRES configuration in general is with 6As in the bifurcation loop, while 7As is acceptable probably due to the rescue by polypyrimidine tract binding protein (PTB) previously described by Kaminiski et al., showing that lengthening the bulge A from 6 As to 7As rendered IRES function dependent on polypyrimidine tract binding protein (PTB). It is possible the vector variants with 4, 5, 8, 10 and 12As also require binding of PBT to the polypyrimidine tract for efficient protein translation and that these vector variants significantly distort the secondary and tertiary structure of the IRES and thus compromise the binding of PBT and/or other trans-acting factors to the polypyrimidine tract, and hence diminish the PBT-mediated rescue of translational activity. Other than the EMCV IRES synthetic constructs made for bicistronic expression vectors, the mutations in the number of adenosine residues in the A-bulge has not been described in EMCV. It seems unlikely that the alterations in number of adenosine residue are driven by any kind of selective pressure, but rather happen during extensive RRV replication over 180 days in the mice, due to its mutation-prone reverse transcriptase activity. In conclusion, in RRVs including the ECMV IRES, it is preferable to use the 6A version of the IRES, not only because of the enhanced transgene expression, but also because of the more frequent direction of oligo A number drift seems to be preferentially towards longer oligo A in the bulge. Thus, if the bulge starts with 6 A's there is more tolerance in terms of transgene expression to the acquisition of a single extra adenosine nucleotide.

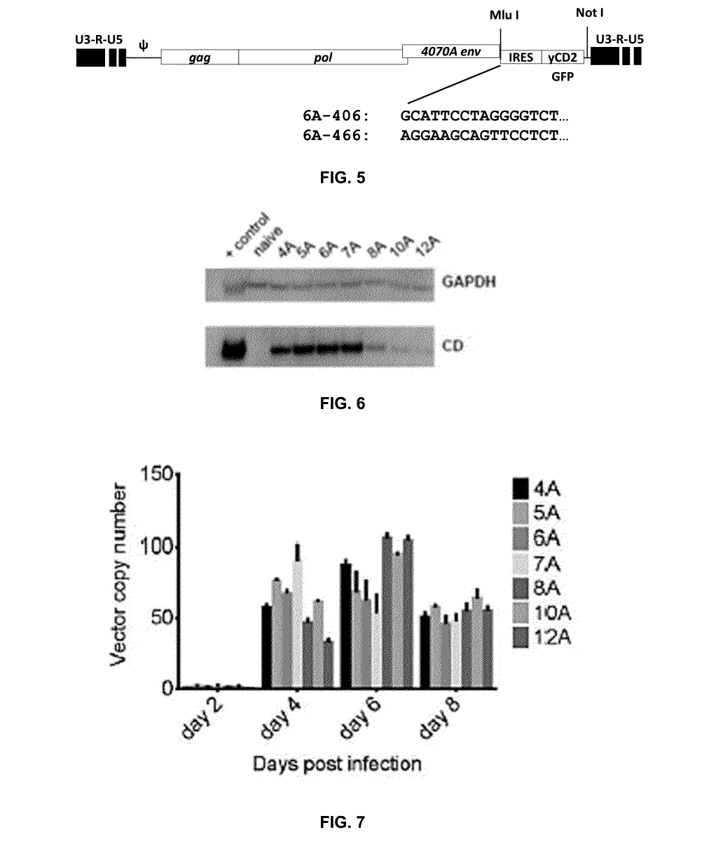

Construction of RRVs containing a minimum IRES with 6A produce similar level of titer, viral transcript and transgene protein expression as the RRV containing the 6A alone. It has been shown that mutants generated by progressive deletion from the 5′ EMCV IRES have differential translational efficiencies in vitro (Duke et al., J Virol. 66:1602-9 1992). Here, RRVs containing various lengths of minimum IRES are generated, designated 6A-406 (e.g., base 123 to 544 of SEQ ID NO:41) and 6A-466 (base 183 to 544 of SEQ ID NO:41) (see, FIG. 5). Other similar constructs with other numbers of A's and either the 406 or 466 IRES sequence can be constructed (designated 7A-406 and 7A-466 (referring to a 7A containing minimal IRES, etc.) and perform approximately in proportion to constructs with the equivalent number of A's and the full length IRES. Each construct is generated by DNA synthesis (BioBasics Inc.) of the entire IRES cassette with a Mlu I at the 5′ end and a Psi I at the 3′end, respectively, for direct replacement of the equivalent cassette in the RRV backbone. All DNA fragments are confirmed by sequencing prior and post cloning into the RRV backbone. The RRV constructs containing the yCD2 transgene were designated using the name of the transgene followed by the number of A's in the A bulge (i.e. yCD2-4A contains yCD2 transgene and 4As in the A bulge in the IRES). The data show that titer from transiently transfected 293T and maximally infected HT1080 cells are similar to that of the bulge A variants. Protein expression of yCD2 is measured from fully infected U87-MG cells. The 6A-406 variant expresses similar level (within 2, 5 or 10 fold) of yCD2 protein in a comparison to the 6A variant with full-length IRES. The 6A-466 variant which carries a further deletion of the 5′ IRES shows expression of yCD2. In addition, data from replication kinetics and vector stability by serial infection also show that both 6A-406 and 6A-466 vectors are stable up to at least 10 cycles of infection.

### Example 2

Intravenous injection of Toca 511 into Balb/C mice. 2.35×10̂6 or 2.35×10̂5 TU of Toca 511 was intravenously administered to 8-week-old female Balb/C mice. Approximately 180 days post infection, genomic DNA from various tissues was harvested for bio-locolization study. Genomic DNA from abnormal tissues such as thymus or lymph node was extracted for sequence analysis of the envelope and IRES-yCD2 cassette.

Construction of RRVs containing various numbers of As in the A bulge of the J-K bifurcation domain. RRVs containing an EMCV IRES and encoding CD or GFP (Ostertag et al., 2012; Perez et al., 2012) were generated to have 4, 5, 6, 7, 8, 10 or 12As in the A bulge in the J-K bifurcation domain. Each construct was generated by DNA synthesis (BioBasics Inc.) of the entire IRES cassette with a Mlu I at the 5′ end and a Psi I at the 3′end, respectively, for direct replacement of the equivalent cassette in the RRV backbone (FIG. 1). All DNA fragments were confirmed by sequencing prior and post cloning into the RRV backbone. The RRV constructs containing the yCD2 transgene were designated using the name of the transgene followed by the number of As in the A bulge (i.e. yCD2-4A contains yCD2 transgene and 4As in the A bulge in the IRES).

Cell Culture. 293T cells were obtained through a materials transfer agreement with the Indiana University Vector Production Facility and Stanford University deposited with ATCC (SD-3515; Lot #2634366). Human glioblastoma cells U87-MG (ATCC, HTB-14), human prostate tumor cells PC-3 (ATCC, CRL-1435) and human fibrosarcoma cells HT-1080 (ATCC, CCL-121) were obtained from ATCC. 293T, U87-MG, PC-3 and HT-1080 cells were cultured in complete DMEM medium containing 10% FBS (Hyclone), sodium pyruvate, glutaMAX (Invitrogen), and antibiotics (penicillin 100 IU/mL, streptomycin 100 IU/mL).

Virus production, infection and titer. Virus stock was first produced by transient transfection of 293T cells using calcium phosphate precipitation method. Cells were seeded at 2×10⁶ cells per 10-cm petri dish the day before transfection. Cells were transfected with 20 μg of designated plasmid DNA the next day. Eighteen hours after transfection, cells were washed with PBS twice and incubated with fresh complete culture medium. Viral supernatant was collected approximately 42 hours post transfection and filtered through a 0.45 μm syringe filter unit. Viral supernatants were stored in aliquots at −80° C. RRV-producer cells were established by infection of HT-1080 cells at equivalent MOI. Viral titers from transiently transfected 293T cells as well as from RRV-producer cells was performed as described (Perez et al., 2012). The viral titers obtained from infected RRV-producer cells were measured before infecting naive U87-MG cells.

Quantification of cellular viral RNA by qRT-PCR. RNA was extracted from naive and RRV-infected U87-MG cells using the RNeasy Kit (Qiagen). Reverse transcription was carried out with 100 ng total RNA using High Capacity cDNA Reverse Transcription Kit (ABI). Quantitative PCR analysis was performed to measure the mRNA expression level of unspliced and spliced cellular viral RNA with the following parameters: 95° C. 10 min; and 40 cycles of 95° C. 15s; 60° C. 30s. The cellular viral RNA expression levels were measured using the primer sets as described above The relative level of RNA from each vector was calculated using 2^{-ΔΔ(Ct)} method with respect to the vector containing the 6As.

Immunoblot and cell-based yCD2 enzymatic assay. Transiently transfected 293T cells or maximally infected U87-MG cells were harvested and lysed for immunoblotting. Equal amount of proteins from lysates were resolved on Criterion XT Precast Gel 4-120 Bis-Tris gels (Bio-Rad, cat #345-0124). Mouse anti-human GAPDH (Millipore cat #MAB374) antibody at 1:500 dilution was used to detect the expression of GAPDH, and mouse anti-yCD2 (Tocagen, clone 9A11) antibody at 1:1,000 dilution was used to detect the expression of yCD2 protein. Detection of protein expression was visualized using Clarity Western ECL Substrate (Bio-Rad, cat #170-5060). Quantity One software (Bio-Rad) was used to quantify the signal of yCD2 and GAPDH detected on the immunoblots. A cell-based enzymatic activity of yCD2 was performed to measure the conversion of 5-FC to 5-FU by high performance liquid chromatography as described (Perez et al., 2012).

Flow cytometry. Cells harvested for flow cytometric analysis were washed with PBS and centrifuged at 1000 rpm for 5 minutes. Cell pellets were resuspended in PBS containing 1% paraformaldehyde. The percentage of GFP-positive cells was determined by flow cytometry using proper gating to exclude GFP-negative cells. Percentage of GFP-positive cells was measured by FACSCanto II using FL1 channel (BD Biosciences). GFP protein expression levels were quantified by using mean fluorescence intensity (MFI).

Vector copy number of proviral DNA. Proviral vector copy numbers in genomic DNA was determined by qPCR as previously described (Perez et al., 2012).

Vector stability assay and amplification of IRES-yCD2 region. Vector stability was measured by serial passage on U87-MG cells as described previously (Perez et al., 2012). PCR was performed using the following primers: 5-127 (forward): 5′-CTGATCTTACTCTTTGGACCTTG-3′ (SEQ ID NO:53) and 3-37 (reverse): 5′-CCCCTTTTTCTGGAGACTAAATAA-3′ (SEQ ID NO:54) which resulted in an ˜1.2-kb fragment. SuperTaq Plus polymerase (Ambion cat #AM2056) was used for all PCR reactions.

PCR and TA cloning for sequence analysis. PCR fragments using the primers and SuperTaq Plus polymerase described were isolated from 0.8% agarose gel and sublconed into TOPO vector provided in the TOPO TA Cloning Kit for Sequencing (Invitrogen, cat #K4530-20). Following selection of bacterial colonies and extraction of plasmid DNA, samples were sequenced using the 5-127 and 3-37 primers. Minimal of 10 colonies of each variants were selected for plasmid DNA extraction and sequencing analysis.

RRV can undergo changes in the length of the oligo adenosine in the A bulge of the EMCV IRES in vivo. The expression of yCD2 and the conversion of 5-FC to 5-FU by yCD2 have been demonstrated to be efficient and stable both in vitro and in vivo when cells are infected with an RRV with a 7A IRES (Toca 511) (Ostertag et al., 2012; Perez et al., 2012). In a vector biolocalization study conducted as part of a preclinical package to support initiation of clinical trials, Toca 511 was injected intravenously into a permissive mouse strain (Balb/c mice) to evaluate long-term vector bio-localization. As expected 10-20% mice (depending on the cohort) at the higher doses (see M&M) displayed abnormalities in lymphoid tissues at 180 days. DNA from the abnormal thymus or lymph nodes of 3 mice were harvested for molecular PCR cloning of proviral sequences, followed by sequencing analysis. One feature was that there were multiple copies of the virus including recombinants with endogenous mouse MCF envelope sequences present, as occurs with lymphomagenesis with ecotropic MLV infection (Fan, 1997). Further analyses are planned for a future publication. However, one additional feature revealed by the sequence analysis of the envelope-IRES-yCD2 transgene cassettes was an expansion or contraction of oligo A sequences in the A bulge of the J-K domain of the IRES in some sequences after presumed extensive viral replication. Tissues from three mice contained vectors with heterogeneous expansions of 7A to 8A, 9A, 10A, 11A and 12A and a contraction of 7A to 6A. It appears that the oligo A number drifts preferentially towards longer oligo A in the A bulge. However, the nature of this preference was undefined in the in vivo study.

Differential transgene expression in RRVs containing various numbers of As in the A bulge in the J-K domain, but similar titers in RRV-producer cells. It has been demonstrated that the J-K domain is important for translational initiation (Duke et al., 1992; Kolupaeva et al., 1998). The observation made from the in vivo study and the 7As in pEMCF as opposed to the 6As in ECMV IRES originally described led to investigate the impact on yCD2 expression of IRESes with various numbers of As in the A bulge, and, in particular, the impact on protein translation in the context of RRV. Therefore, a series of deletion and insertion mutants were generated specifically in the A bulge to mimic mutations observed from the in vivo study. RRVs containing an EMCV IRES and encoding yCD2 or GFP were generated to have 4, 5, 6, 7, 8, 10 or 12As in the A bulge in the J-K bifurcation domain (FIG. 1B). yCD2 and GFP protein expression mediated by IRES variants in transiently transfected 293T cells were analyzed. The data showed that yCD2 protein expression levels mediated by RRV variants containing 5 and 6A were comparable to that of the 7A. In contrast, yCD2 protein expression levels mediated by RRV variants containing 4, 8, 10 and 12A were substantially reduced (FIG. 6). A similar result was observed with IRES variants expressing the GFP transgene when comparing their mean fluorescent intensity levels.

Next the alteration in the A bulge was examined to see fi there would be an affect viral titer. Virus stocks were initially produced by transient transfection in 293T cells, followed by infection of HT-1080 cells at multiplicity of infection (MOI) of 0.1 to generate RRV-producer cells. FIG. 1C shows that RRV containing various number of As from transiently transfected 293T cells produced similar titers. The viral titer of each vector produced by the RRV-producer HT1080 cells in the subsequent infection was also determined. Similar to viral titer data obtained from transiently transfected 293T cells, RRV-producer cells containing various numbers of As also produced comparable titers (FIG. 1C), suggesting that the number of the As in the A bulge does not affect viral titer.

RRVs containing various number of As in the A bulge replicate at similar rate. Given that the number of the As in the A bulge does not affect viral titer produced from cells initially infected with low MOI, it is likely that these vectors also replicate at similar rates. The replication kinetics of these vectors were analyzed by measuring the average vector number during the course of infection. Viral supernatants from RRV-producer cells were used to infect naive U87-MG cells at MOI of 0.01. At each passage a portion of cells were harvested for genomic DNA extraction for qPCR analysis. FIG. 7 shows that the vector copy number varied among vectors at day 4 and day 6 post infection and stabilized by day 8 post infection with comparable the average vector copy numbers.

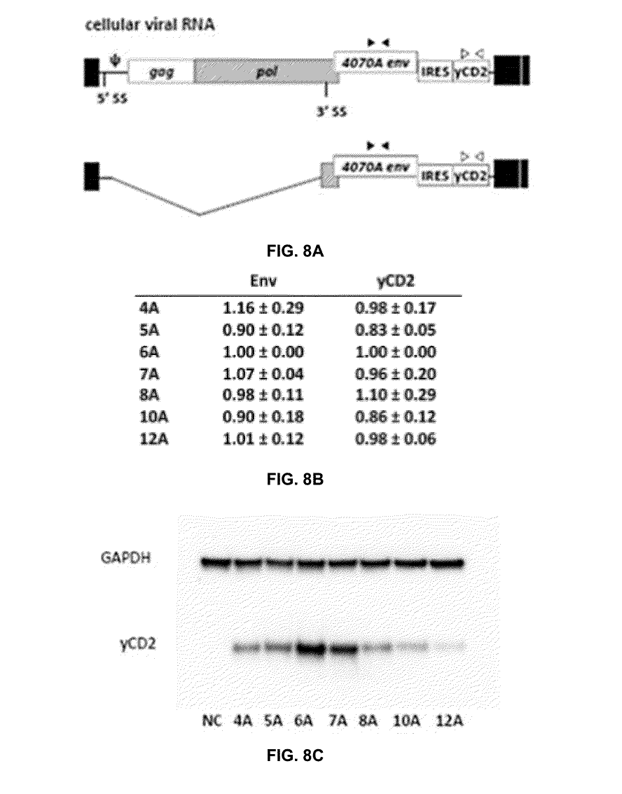

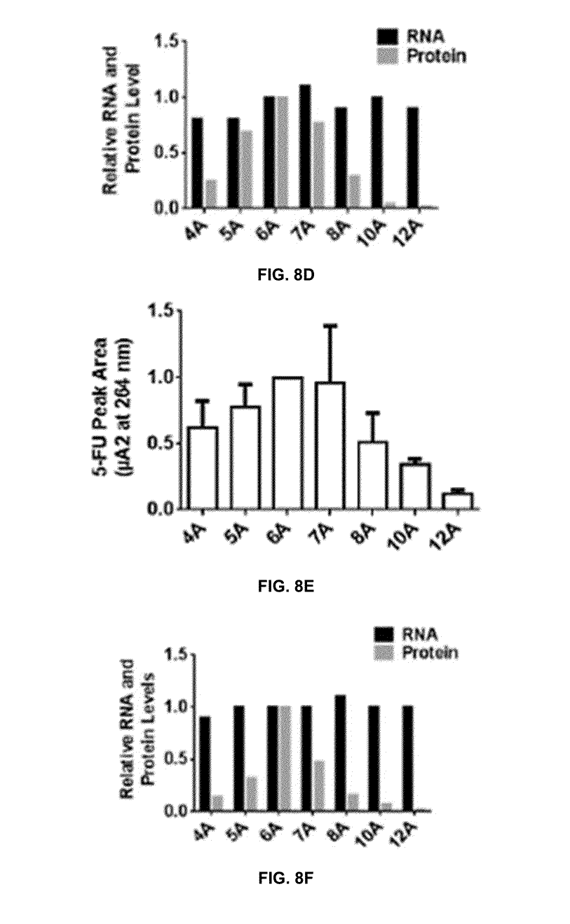

RRVs containing various numbers of As in the J-K bifurcation domain express similar levels of transcripts but different levels of protein. The yCD2 protein expression from transiently transfected 293T cells was substantially less from vectors carrying the 8, 10 or 12As than those carrying the 4, 5, 6 and 7As (FIG. 6). In order to demonstrate that the decrease in transgene expression mediated by IRES variants is regulated at the translational level, cellular viral RNA levels of fully infected U87-MG cells were harvested and measured by quantitative real-time polymerase chain reaction (qRT-PCR), and yCD2 protein levels were examined by immunoblotting (Perez et al., 2012). The cellular viral RNA levels were measured using two different primer sets, located in the env and in yCD2 region, respectively, (FIG. 8A). The relative level of RNA from each vector was calculated using the 2-^{ΔΔ(Ct)} method with respect to the vector containing 6As. The cellular viral RNA level ratios ranged from 0.9 to 1.2, and the value of ratios from each primer set were comparable (FIG. 8B). Together, the data suggest that there is no significant difference in viral RNA transcript levels due to the modifications in the IRES. In examining the yCD2 protein expression level of these vectors by Western blot, showed that the yCD2 protein expression levels of the vectors containing the 5 and 7As were 69% and 77% that of the yCD2-6A vector. In contrast, a substantial reduction of yCD2 protein expression was observed in the vectors containing the 4, 8, 10 and 12As. The CD protein expression levels of these vectors range from 4 to 25% that of the yCD2-6A vector (FIG. 8C). The drastic reduction of the yCD2 protein expression with similar expression levels of the cellular viral RNA (FIG. 8D) suggest that the length of oligo A in the bulge A of the IRES can have a large effect on protein expression at the post-transcriptional level.

Relative intracellular yCD2 enzymatic activity was also measured, employing a cell-based assay by adding 5-FC to the cultures and measuring 5-FU after an hour by high performance liquid chromatography (HPLC). The differences in activity were ranked similarly to the Western blot data, (FIG. 8E) and a correlation (R²=0.8995) was observed between yCD2 expression and enzymatic activity. To confirm the generality of the observations with yCD2 gene, we measured the effect of the number of As in the A bulge with another transgene for which the protein expression assay was well defined.

Therefore, an equivalent set of RRVs encoding GFP were generated. The RNA expression levels of these vectors were comparable to one another. Consistent with the data observed in yCD2 vectors, a substantial reduction of GFP protein expression was observed in vectors containing the 4, 8, 10 and 12As with minimal change at the viral RNA level (FIG. 8F). Overall, the results from GFP vectors were consistent with those observed with yCD2 vectors, and the vectors containing the 6As express the highest level of protein from the transgene in both sets of vectors. Furthermore, due to the sensitivity of the detection method, a remarkable difference in GFP expression level was revealed, showing approximately 96% and 99% decrease in GFP expressed by the vectors containing the 10As and 12As, respectively. In both sets of the vectors, RRV with 7As showed an approximately 30% decrease in protein expression compared to 6As. The reduced protein translation efficiency in RRV with 4As and 5As compared to RRV with 6As is also consistent with findings of the mutant 868Δ4 reported by Hoffman et al. (Hoffman and Palmenberg, 1995).

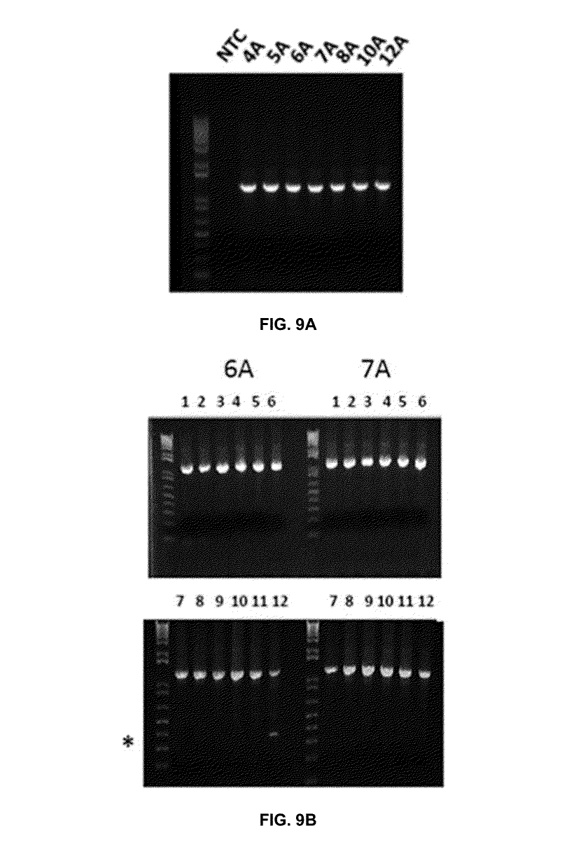

RRVs containing 6As and 7As in the A bulge exhibit similar vector stability. To ensure that the reduction in yCD2 protein expression in RRV with 4As, 10As and 12As is not due to deletion in the IRES-yCD2 cassette outside region of which the yCD2 primer set binds in qRT-PCR, the vector stability was examined in two different settings. In one setting, the viral supernatant from RRV-producer cells was used to infect naïve U87-MG cells at MOI of 0.01 to allow time for the virus to replicate to day 10 to match the time points of the samples harvested for qRT-PCR and immunoblotting. The genomic DNA of infected cells was isolated and amplified to obtain a 1.2 kb PCR product of the proviral DNA to assess the integrity of the integrated viral genome (FIG. 1B) as previously described (Logg et al., 2002; Perez et al., 2012). No detection of deletion mutants (PCR products<1.2 kb represent partial or complete deletion of viral genome in the IRES-yCD2 region) was observed (FIG. 9A). Together the data indicate that the vectors are stable in such a short-term replication setting and the reduction of yCD2 protein expression is not due to deletion in the IRES-yCD2 cassette.

Since RRV carrying the 6A appears to have higher protein expression than the one carrying the 7A, a comparison of their long-term vector stability was performed. The same experiment was performed over serial infection cycles by collecting viral supernatant from fully infected U87-MG cells, infecting fresh U87-MG cells for 12 cycles, harvesting the genomic DNA after each infection and amplifying a 1.2 kb PCR product to assess the integrity of the integrated viral genome. PCR result showed that both vectors were completely stable up to infection cycle 11. At infection cycle 12, emergence of deletion mutants, indicated by a PCR product of approximately 0.25 kb, was observed in the vector with 6As. However, the 1.2 kb band carrying the intact IRES-GFP region could still be detected at infection cycle 12 (FIG. 9B). As generation of these deletions appears to be a stochastic process, it is likely that the 6A and 7A vectors have roughly equivalent stabilities after serial replication.

In vitro viral replication and analysis of mutations in the A bulge of RRVs carrying various numbers of As. In order to mimic the in vivo study in which extensive rounds of viral replication occurred and length variation in the A bulge was observed, in vitro replication experiments were performed to examine the viral genomic stability of these vectors particularly in the A bulge. It has been reported that repeat of As in DNA template can produce artifacts in PCR when using Taq DNA polymerase (Shinde et al., 2003) even though it contains a proofreading activity. To ensure that expansion of oligo A in the A bulge observed in vivo previously and in vitro replication described below is not contributed by such an event, PCR was performed using plasmid DNA as the template. Sequence analysis from PCR cloning using plasmid DNA with 4, 5, 6, 7, and 8As as template did not produce any mutation. In contrast, plasmid DNA carrying the 10As variant resulted in 1 clone that showed contraction to 9A. Likewise, the 12A variant gave rise to 1 clone that showed contraction to 8As. The data indicate that the Taq polymerase effect is minimal and appears to favor contraction; they are consistent with Shinde et al., in which they reported no mutations observed even after 60 PCR cycles for (A)_{r.} with eight or less repeat units (Shinde et al., 2003).

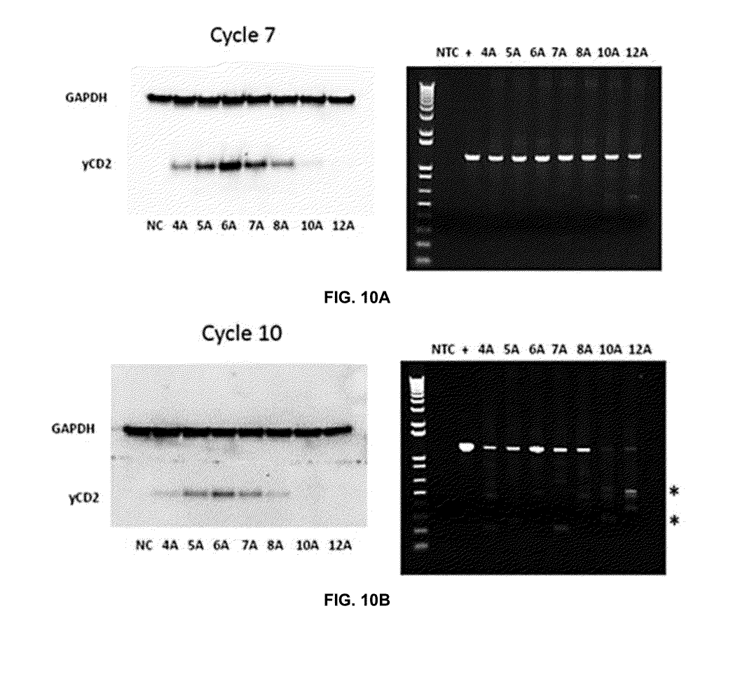

After confirming that PCR artifact is minimal, serial infection cycles were performed and cells at indicated infection cycles were harvested to examine the changes in the yCD2 protein expression from cell lysates and the length of As in the A bulge in proviral DNA by immunoblotting and by TA cloning of the PCR product, respectively. The expression of yCD2 was compared between infection cycle 1 and 7. The expression levels of yCD2 among the RRVs carrying various numbers of As from infection cycle 1 was consistent with data shown previously in FIG. 8C. After 7 cycles of infection in vitro, the yCD2 expression in RRVs carrying the 10As and 12As was substantially reduced (FIG. 10A). Notably, the reduction in yCD2 expression observed in 10As and 12As variants is not predominantly due to deletion in the IRES-yCD2 cassette as evident by the PCR result (FIG. 10A). In parallel, sequence analysis was performed to examine changes that might have occurred in the A bulge after 7 cycles of infection. Sequence analysis revealed that the length of As in variants carrying the 4As and 5As remained 100% stable. Variants carrying the 6As and 7As remained relatively stable. Eight out of ten clones from the 6A variant remained the same length; two out of ten clones showed expansion to 7As. For the variant carrying 7As, 6/10 clones remained the same length whereas others expanded to 8As and 10As. For the variant carrying the 8As, 3/10 clones remained the same length. In addition, a range of expansion from 9As to 22As was observed. Interesting 1/10 clones showed a contraction to 7As. However, the length of expansion does not appear to measurably affect the overall yCD2 expression (FIG. 10A; compare to FIG. 8C). In contrast, variants originally carrying the 10As and 12As, both had extensively expanded to As ranging from 12As to 54As and these expansions correlate with a substantial reduction in yCD2 expression (FIG. 10A; compare to FIG. 8C). Furthermore, data from infection cycle 10 indicate that, while trace deletions in the IRES-yCD2 cassette could be detected in variants with 4 to 8 As, variants with 10 and 12As had mostly deleted sequences in the IRES-yCD2 (FIG. 10B). However, the length of oligo A in the A bulge in variants with 4 to 8As remained roughly stable after infection cycle 7; the variants carrying the 4As and 5As continue to remain stable over time; the variant carrying the 6As showed 2/10 clones expanded to 7As by infection cycle 10; and the variant carrying the 7As was also relatively stable and did not show further expansion of the proportion of mutations from that observed in infection cycle 7. While the 8A variant also did not change the proportion of mutants from 7 to 10 cycles, it had already incorporated more mutations at cycle 7. In addition, the expansion of oligo A in variants carrying the 10As and 12As appears to have compromised the viral genome stability as indicated by the deletion of the IRES-yCD2 cassette in PCR. In contrast to data from infection cycle 7 for the 10 and 12A variants, in which the reduction of yCD2 expression appears to associated with expansion of the oligo A in the A bulge, the reduction of yCD2 expression in cycle 10 is presumably due mainly to the emergence of deletion mutants, on top of the oligo A expansion.

A number of embodiments of the disclosure have been described. Nevertheless, it will be understood that various modifications may be made without departing from the spirit and scope of the disclosure. Accordingly, other embodiments are within the scope of the following claims.

What is claimed is:

1. An engineered nucleic acid comprising an Internal Ribsome Entry Site (IRES) having 5As in the A-bulge of the J-K bifurcation region.

2. A recombinant vector, comprising and internal ribosome entry site (IRES) comprising a sequence selected from the group consisting of:
(i) a sequence having 95% identity to SEQ ID NO:41 and having 5-6A's in the J-K bifurcation region;
(ii) a truncated IRES comprising a sequence as set forth in SEQ ID NO:41 containing 5A's in the bifurcation region and having a sequence beginning between nucleotide 1 to about 183 and continues to nucleotide 544 of SEQ ID NO:41;
(iii) a truncated IRES comprising a sequence as set forth in SEQ ID NO:41 from about nucleotide 123 to nucleotide 544 or from about nucleotide 183 to 544 and having 5As in the A-bulge of the J-K bifurcation region wherein the vector comprises improved stability compared to an IRES with 7As in the bifurcation region;
(iv) a sequence as set forth in SEQ ID NO:41 having 5As in the A-bulge of the J-K bifurcation region; and
(v) any of the foregoing wherein T can be U.

3. A recombinant replication competent retrovirus comprising:
a retroviral GAG protein;
a retroviral POL protein;
a retroviral envelope;
a retroviral polynucleotide comprising Long-Terminal Repeat (LTR) sequences at the 3′ end of the retroviral polynucleotide sequence, a promoter sequence at the 5′ end of the retroviral polynucleotide, said promoter being suitable for expression in a mammalian cell, a gag nucleic acid domain, a pol nucleic acid domain and an env nucleic acid domain;
a cassette comprising an internal ribosome entry site (IRES) consisting of 5 or 6A's in the A-bulge in the bifurcation region of the IRES, wherein the IRES is operably linked to a heterologous polynucleotide, wherein the cassette is positioned 5′ to the 3′ LTR and 3′ to the env nucleic acid domain encoding the retroviral envelope; and
cis-acting sequences necessary for reverse transcription, packaging and integration in a target cell.

4. The recombinant replication competent retrovirus of claim 3, wherein the retroviral polynucleotide sequence is derived from a virus selected from the group consisting of murine leukemia virus (MLV), Moloney murine leukemia virus (MoMLV), Feline leukemia virus (FeLV), Baboon endogenous retrovirus (BEV), porcine endogenous virus (PERV), the cat derived retrovirus RD114, squirrel monkey retrovirus, Xenotropic murine leukemia virus-related virus(XMRV), avian reticuloendotheliosis virus(REV), or Gibbon ape leukemia virus (GALV).

5. The recombinant replication competent retrovirus of claim 3, wherein the retroviral envelope is an amphotropic MLV envelope.

6. The recombinant replication competent retrovirus of claim 3, wherein the target cell is a neoplastic cell.

7. The recombinant replication competent retrovirus of claim 3, wherein the promoter sequence is (i) a promoter from a growth regulatory gene; (ii) a tissue specific promoter; or (iii) a CMV promoter.

8. The recombinant replication competent retrovirus of claim 7, wherein the tissue-specific promoter sequence comprises at least one androgen response element (ARE).

9. The recombinant replication competent retrovirus of claim 3, wherein the IRES consists of the sequence set forth in SEQ ID NO:41.

10. The recombinant replication competent retrovirus of claim 3, wherein the retroviral polynucleotide sequence comprises (i) the sequence set forth in SEQ ID NO:42 or (ii) the sequence as set forth in SEQ ID NO:42, wherein T is U.

11. The recombinant replication competent retrovirus of claim 3, wherein the heterologous nucleic acid encodes a polypeptide having cytosine deaminase or thymidine kinase activity.

12. The recombinant replication competent retrovirus of claim 3, wherein the heterologous nucleic acid is human codon optimized and encodes a polypeptide as set forth in SEQ ID NO:4.

13. The recombinant replication competent retrovirus of claim 3, wherein the heterologous nucleic acid comprises a sequence as set forth in SEQ ID NO: 19 or 22 from about nucleotide number 8877 to about 9353.

14. The recombinant replication competent retrovirus of claim 3, wherein the heterologous nucleic acid sequence encodes a biological response modifier or an immunopotentiating cytokine.

15. The recombinant replication competent retrovirus of claim 14, wherein the immunopotentiating cytokine is selected from the group consisting of interleukins 1 through 15, interferon, tumor necrosis factor (TNF), and granulocyte-macrophage-colony stimulating factor (GM-CSF).

16. The recombinant replication competent retrovirus of claim 14, wherein the immunopotentiating cytokine is interferon gamma.

17. The recombinant replication competent retrovirus of claim 3, wherein the heterologous nucleic acid encodes a polypeptide that converts a nontoxic prodrug in to a toxic drug.

18. A recombinant retroviral polynucleotide genome for producing a retrovirus of claim 3.

19. A method of treating a cell proliferative disorder comprising contacting the subject with a recombinant replication competent retrovirus of claim 11 under conditions such that the cytosine deaminase polynucleotide is expressed and contacting the subject with 5-fluorocytosine.

20. The method of claim 19, wherein the cell proliferative disorder is glioblastoma multiforme.

21. The method of claim 19, wherein the cell proliferative disorder is selected from the group consisting of lung cancer, colon-rectum cancer, breast cancer, prostate cancer, urinary tract cancer, uterine cancer, brain cancer, head and neck cancer, pancreatic cancer, melanoma, stomach cancer and ovarian cancer.

22. A vector that expresses a heterologous gene in a mammalian cell from an internal ribosome entry site consisting of 5 or 6As in the A bulge in the J-K bifurcation region.

23. The vector of claim 22, wherein the vector is a viral vector.

24. The vector of claim 23, wherein the vector is a retroviral replicating vector.

25. The vector of claim 24, wherein the vector is derived from a gamma-retrovirus.

26. The vector of claim 25, wherein the gamma-retrovirus is a Murine Leukemia Virus, Baboon Endogenous Virus, Gibbon Ape Leukemia virus, or Feline leukemia virus.

27. The vector of claim 22, wherein the heterologous gene is a gene with a therapeutic activity in mammals.

28. The vector of claim 27, wherein the therapeutic activity is an anticancer activity.

29. A method of treating cancer, by administering the vector of claim 28.

30. A recombinant replication competent retrovirus comprising:
a retroviral GAG protein;
a retroviral POL protein;
a retroviral envelope;
a retroviral polynucleotide comprising Long-Terminal Repeat (LTR) sequences at the 3′ end of the retroviral polynucleotide sequence, a promoter sequence at the 5′ end of the retroviral polynucleotide, said promoter being suitable for expression in a mammalian cell, a gag nucleic acid domain, a pol nucleic acid domain and an env nucleic acid domain;
a cassette comprising (i) a minimal internal ribosome entry site (IRES), wherein the minimal IRES is operably linked to a heterologous polynucleotide, (ii) a cassette of (i) and a polIII promoter linked to an inhibitory nucleic acid, or (iii) a cassetee of (i) and a mini-promoter operably linked to a heterologous polynucleotide, wherein the cassette is positioned 5′ to the 3′ LTR and 3′ to the env nucleic acid domain encoding the retroviral envelope; and
cis-acting sequences necessary for reverse transcription, packaging and integration in a target cell.

31. The replication competent retrovirus of claim 30, wherein the minimal IRES consists of a sequence from about nucleotide 123 or 183 to 544 of SEQ ID NO:41.

32. The replication competent retrovirus of claim 30, wherein the minimal IRES consists of 5 or 6As in the A bulge.

33. The recombinant replication competent retrovirus of claim 30, wherein the retroviral polynucleotide sequence is derived from a virus selected from the group consisting of murine leukemia virus (MLV), Moloney murine leukemia virus (MoMLV), Feline leukemia virus (FeLV), Baboon endogenous retrovirus (BEV), porcine endogenous virus (PERV), the cat derived retrovirus RD114, squirrel monkey retrovirus, Xenotropic murine leukemia virus-related virus(XMRV), avian reticuloendotheliosis virus(REV), or Gibbon ape leukemia virus (GALV).

34. The recombinant replication competent retrovirus of claim 30, wherein the retroviral envelope is an amphotropic MLV envelope.

35. The recombinant replication competent retrovirus of claim 30, wherein the heterologous nucleic acid encodes a polypeptide having thymidine kinase, purine nucleoside phosphorylase (PNP), or cytosine deaminase activity.

36. The recombinant replication competent retrovirus of claim 30, wherein the inhibitory polynucleotide comprises an miRNA, RNAi or siRNA sequence.

37. A recombinant retroviral polynucleotide genome for producing a retrovirus of claim 30.
